SEQUENCE LISTING (25 sequences)

SEQ ID NO: 1 - protein, 1331 aa, Homo sapiens
TVAAGCPDQSPELQPWNPGHDQDHHVHIGQGKTLLLTSSATVYSIHISEGGKLVIKDHDEPIVLRTRHILIDNGGELHAGSALCPFQGNFTIILYGRADEGIQPDPYYGLKYIGVGKGGALELHGQKKLSWTFLNKTLHPGGMAEGGYFFERSWGHRGVIVHVIDPKSGTVIHSDRFDTYRSKKESERLVQYLNAVPDGRILSVAVNDEGSRNLDDMARKAMTKLGSKHFLHLGFRHPWSFLTVKGNPSSSVEDHIEYHGHRGSAAARVFKLFQTEHGEYFNVSLSSEWVQDVEWTEWFDHDKVSQTKGGEKISDLWKAHPGKICNRPIDIQATTMDGVNLSTEVVYKKGQDYRFACYDRGRACRSYRVRFLCGKPVRPKLTVTIDTNVNSTILNLEDNVQSWKPGDTLVIASTDYSMYQAEEFQVLPCRSCAPNQVKVAGKPMYLHIGEEIDGVDMRAEVGLLSRNIIVMGEMEDKCYPYRNHICNFFDFDTFGGHIKFALGFKAAHLEGTELKHMGQQLVGQYPIHFHLAGDVDERGGYDPPTYIRDLSIHHTFSRCVTVHGSNGLLIKDVVGYNSLGHCFFTEDGPEERNTFDHCLGLLVKSGTLLPSDRDSKMCKMITEDSYPGYIPKPRQDCNAVSTFWMANPNNNLINCAAAGSEETGFWFIFHHVPTGPSVGMYSPGYSEHIPLGKFYNNRAHSNYRAGMIIDNGVKTTEASAKDKRPFLSIISARYSPHQDADPLKPREPAIIRHFIAYKNQDHGAWLRGGDVWLDSCRFADNGIGLTLASGGTFPYDDGSKQEIKNSLFVGESGNVGTEMMDNRIWGPGGLDHSGRTLPIGQNFPIRGIQLYDGPINIQNCTFRKFVALEGRHTSALAFRLNNAWQSCPHNNVTGIAFEDVPITSRVFFGEPGPWFNQLDMDGDKTSVFHDVDGSVSEYPGSYLTKNDNWLVRHPDCINVPDWRGAICSGCYAQMYIQAYKTSNLRMKIIKNDFPSHPLYLEGALTRSTHYQQYQPVVTLQKGYTIHWDQTAPAELAIWLINFNKGDWIRVGLCYPRGTTFSILSDVHNRLLKQTSKTGVFVRTLQMDKVEQSYPGRSHYYWDEDSGLLFLKLKAQNEREKFAFCSMKGCERIKIKALIPKNAGVSDCTATAYPKFTERAVVDVPMPKKLFGSQLKTKDHFLEVKMESSKQHFFHLWNDFAYIEVDGKKYPSSEDGIQVVVIDGNQGRVVSHTSFRNSILQGIPWQLFNYVATIPDNSIVLMASKGRYVSRGPWTRVLEKLGADRGLKLKEQMAFVGFKGSFRPIWVTLDTEDHKAKIFQVVPIPVVKKKKL

SEQ ID NO: 2 - protein, 1328 aa, Homo sapiens
AGCPDQSPELQPWNPGHDQDHHVHIGQGKTLLLTSSATVYSIHISEGGKLVIKDHDEPIVLRTRHILIDNGGELHAGSALCPFQGNFTIILYGRADEGIQPDPYYGLKYIGVGKGGALELHGQKKLSWTFLNKTLHPGGMAEGGYFFERSWGHRGVIVHVIDPKSGTVIHSDRFDTYRSKKESERLVQYLNAVPDGRILSVAVNDEGSRNLDDMARKAMTKLGSKHFLHLGFRHPWSFLTVKGNPSSSVEDHIEYHGHRGSAAARVFKLFQTEHGEYFNVSLSSEWVQDVEWTEWFDHDKVSQTKGGEKISDLWKAHPGKICNRPIDIQATTMDGVNLSTEVVYKKGQDYRFACYDRGRACRSYRVRFLCGKPVRPKLTVTIDTNVNSTILNLEDNVQSWKPGDTLVIASTDYSMYQAEEFQVLPCRSCAPNQVKVAGKPMYLHIGEEIDGVDMRAEVGLLSRNIIVMGEMEDKCYPYRNHICNFFDFDTFGGHIKFALGFKAAHLEGTELKHMGQQLVGQYPIHFHLAGDVDERGGYDPPTYIRDLSIHHTFSRCVTVHGSNGLLIKDVVGYNSLGHCFFTEDGPEERNTFDHCLGLLVKSGTLLPSDRDSKMCKMITEDSYPGYIPKPRQDCNAVSTFWMANPNNNLINCAAAGSEETGFWFIFHHVPTGPSVGMYSPGYSEHIPLGKFYNNRAHSNYRAGMIIDNGVKTTEASAKDKRPFLSIISARYSPHQDADPLKPREPAIIRHFIAYKNQDHGAWLRGGDVWLDSCRFADNGIGLTLASGGTFPYDDGSKQEIKNSLFVGESGNVGTEMMDNRIWGPGGLDHSGRTLPIGQNFPIRGIQLYDGPINIQNCTFRKFVALEGRHTSALAFRLNNAWQSCPHNNVTGIAFEDVPITSRVFFGEPGPWFNQLDMDGDKTSVFHDVDGSVSEYPGSYLTKNDNWLVRHPDCINVPDWRGAICSGCYAQMYIQAYKTSNLRMKIIKNDFPSHPLYLEGALTRSTHYQQYQPVVTLQKGYTIHWDQTAPAELAIWLINFNKGDWIRVGLCYPRGTTFSILSDVHNRLLKQTSKTGVFVRTLQMDKVEQSYPGRSHYYWDEDSGLLFLKLKAQNEREKFAFCSMKGCERIKIKALIPKNAGVSDCTATAYPKFTERAVVDVPMPKKLFGSQLKTKDHFLEVKMESSKQHFFHLWNDFAYIEVDGKKYPSSEDGIQVVVIDGNQGRVVSHTSFRNSILQGIPWQLFNYVATIPDNSIVLMASKGRYVSRGPWTRVLEKLGADRGLKLKEQMAFVGFKGSFRPIWVTLDTEDHKAKIFQVVPIPVVKKKKL

SEQ ID NO: 3 - protein, 1361 aa, Homo sapiens
MGAAGRQDFLFKAMLTISWLTLTCFPGATSTVAAGCPDQSPELQPWNPGHDQDHHVHIGQGKTLLLTSSATVYSIHISEGGKLVIKDHDEPIVLRTRHILIDNGGELHAGSALCPFQGNFTIILYGRADEGIQPDPYYGLKYIGVGKGGALELHGQKKLSWTFLNKTLHPGGMAEGGYFFERSWGHRGVIVHVIDPKSGTVIHSDRFDTYRSKKESERLVQYLNAVPDGRILSVAVNDEGSRNLDDMARKAMTKLGSKHFLHLGFRHPWSFLTVKGNPSSSVEDHIEYHGHRGSAAARVFKLFQTEHGEYFNVSLSSEWVQDVEWTEWFDHDKVSQTKGGEKISDLWKAHPGKICNRPIDIQATTMDGVNLSTEVVYKKGQDYRFACYDRGRACRSYRVRFLCGKPVRPKLTVTIDTNVNSTILNLEDNVQSWKPGDTLVIASTDYSMYQAEEFQVLPCRSCAPNQVKVAGKPMYLHIGEEIDGVDMRAEVGLLSRNIIVMGEMEDKCYPYRNHICNFFDFDTFGGHIKFALGFKAAHLEGTELKHMGQQLVGQYPIHFHLAGDVDERGGYDPPTYIRDLSIHHTFSRCVTVHGSNGLLIKDVVGYNSLGHCFFTEDGPEERNTFDHCLGLLVKSGTLLPSDRDSKMCKMITEDSYPGYIPKPRQDCNAVSTFWMANPNNNLINCAAAGSEETGFWFIFHHVPTGPSVGMYSPGYSEHIPLGKFYNNRAHSNYRAGMIIDNGVKTTEASAKDKRPFLSIISARYSPHQDADPLKPREPAIIRHFIAYKNQDHGAWLRGGDVWLDSCRFADNGIGLTLASGGTFPYDDGSKQEIKNSLFVGESGNVGTEMMDNRIWGPGGLDHSGRTLPIGQNFPIRGIQLYDGPINIQNCTFRKFVALEGRHTSALAFRLNNAWQSCPHNNVTGIAFEDVPITSRVFFGEPGPWFNQLDMDGDKTSVFHDVDGSVSEYPGSYLTKNDNWLVRHPDCINVPDWRGAICSGCYAQMYIQAYKTSNLRMKIIKNDFPSHPLYLEGALTRSTHYQQYQPVVTLQKGYTIHWDQTAPAELAIWLINFNKGDWIRVGLCYPRGTTFSILSDVHNRLLKQTSKTGVFVRTLQMDKVEQSYPGRSHYYWDEDSGLLFLKLKAQNEREKFAFCSMKGCERIKIKALIPKNAGVSDCTATAYPKFTERAVVDVPMPKKLFGSQLKTKDHFLEVKMESSKQHFFHLWNDFAYIEVDGKKYPSSEDGIQVVVIDGNQGRVVSHTSFRNSILQGIPWQLFNYVATIPDNSIVLMASKGRYVSRGPWTRVLEKLGADRGLKLKEQMAFVGFKGSFRPIWVTLDTEDHKAKIFQVVPIPVVKKKKL

SEQ ID NO: 4 - DNA, 4171 bp, Homo sapiens
cgtgacactgtctcggctacagacccagagggagcacactgccaggatgggagctgctgggaggcaggacttcctcttcaaggccatgctgaccatcagctggctcactctgacctgcttccctggggccacatccacagtggctgctgggtgccctgaccagagccctgagttgcaaccctggaaccctggccatgaccaagaccaccatgtgcatatcggccagggcaagacactgctgctcacctcttctgccacggtctattccatccacatctcagagggaggcaagctggtcattaaagaccacgacgagccgattgttttgcgaacccggcacatcctgattgacaacggaggagagctgcatgctgggagtgccctctgccctttccagggcaatttcaccatcattttgtatggaagggctgatgaaggtattcagccggatccttactatggtctgaagtacattggggttggtaaaggaggcgctcttgagttgcatggacagaaaaagctctcctggacatttctgaacaagacccttcacccaggtggcatggcagaaggaggctatttttttgaaaggagctggggccaccgtggagttattgttcatgtcatcgaccccaaatcaggcacagtcatccattctgaccggtttgacacctatagatccaagaaagagagtgaacgtctggtccagtatttgaacgcggtgcccgatggcaggatcctttctgttgcagtgaatgatgaaggttctcgaaatctggatgacatggccaggaaggcgatgaccaaattgggaagcaaacacttcctgcaccttggatttagacacccttggagttttctaactgtgaaaggaaatccatcatcttcagtggaagaccatattgaatatcatggacatcgaggctctgctgctgcccgggtattcaaattgttccagacagagcatggcgaatatttcaatgtttctttgtccagtgagtgggttcaagacgtggagtggacggagtggttcgatcatgataaagtatctcagactaaaggtggggagaaaatttcagacctctggaaagctcacccaggaaaaatatgcaatcgtcccattgatatacaggccactacaatggatggagttaacctcagcaccgaggttgtctacaaaaaaggccaggattataggtttgcttgctacgaccggggcagagcctgccggagctaccgtgtacggttcctctgtgggaagcctgtgaggcccaaactcacagtcaccattgacaccaatgtgaacagcaccattctgaacttggaggataatgtacagtcatggaaacctggagataccctggtcattgccagtactgattactccatgtaccaggcagaagagttccaggtgcttccctgcagatcctgcgcccccaaccaggtcaaagtggcagggaaaccaatgtacctgcacatcggggaggagatagacggcgtggacatgcgggcggaggttgggcttctgagccggaacatcatagtgatgggggagatggaggacaaatgctacccctacagaaaccacatctgcaatttctttgacttcgatacctttgggggccacatcaagtttgctctgggatttaaggcagcacacttggagggcacggagctgaagcatatgggacagcagctggtgggtcagtacccgattcacttccacctggccggtgatgtagacgaaaggggaggttatgacccacccacatacatcagggacctctccatccatcatacattctctcgctgcgtcacagtccatggctccaatggcttgttgatcaaggacgttgtgggctataactctttgggccactgcttcttcacggaagatgggccggaggaacgcaacacttttgaccactgtcttggcctccttgtcaagtctggaaccctcctcccctcggaccgtgacagcaagatgtgcaagatgatcacagaggactcctacccagggtacatccccaagcccaggcaagactgcaatgctgtgtccaccttctggatggccaatcccaacaacaacctcatcaactgtgccgctgcaggatctgaggaaactggattttggtttatttttcaccacgtaccaacgggcccctccgtgggaatgtactccccaggttattcagagcacattccactgggaaaattctataacaaccgagcacattccaactaccgggctggcatgatcatagacaacggagtcaaaaccaccgaggcctctgccaaggacaagcggccgttcctctcaatcatctctgccagatacagccctcaccaggacgccgacccgctgaagccccgggagccggccatcatcagacacttcattgcctacaagaaccaggaccacggggcctggctgcgcggcggggatgtgtggctggacagctgccggtttgctgacaatggcattggcctgaccctggccagtggtggaaccttcccgtatgacgacggctccaagcaagagataaagaacagcttgtttgttggcgagagtggcaacgtggggacggaaatgatggacaataggatctggggccctggcggcttggaccatagcggaaggaccctccctataggccagaattttccaattagaggaattcagttatatgatggccccatcaacatccaaaactgcactttccgaaagtttgtggccctggagggccggcacaccagcgccctggccttccgcctgaataatgcctggcagagctgcccccataacaacgtgaccggcattgcctttgaggacgttccgattacttccagagtgttcttcggagagcctgggccctggttcaaccagctggacatggatggggataagacatctgtgttccatgacgtcgacggctccgtgtccgagtaccctggctcctacctcacgaagaatgacaactggctggtccggcacccagactgcatcaatgttcccgactggagaggggccatttgcagtgggtgctatgcacagatgtacattcaagcctacaagaccagtaacctgcgaatgaagatcatcaagaatgacttccccagccaccctctttacctggagggggcgctcaccaggagcacccattaccagcaataccaaccggttgtcaccctgcagaagggctacaccatccactgggaccagacggcccccgccgaactcgccatctggctcatcaacttcaacaagggcgactggatccgagtggggctctgctacccgcgaggcaccacattctccatcctctcggatgttcacaatcgcctgctgaagcaaacgtccaagacgggcgtcttcgtgaggaccttgcagatggacaaagtggagcagagctaccctggcaggagccactactactgggacgaggactcagggctgttgttcctgaagctgaaagctcagaacgagagagagaagtttgctttctgctccatgaaaggctgtgagaggataaagattaaagctctgattccaaagaacgcaggcgtcagtgactgcacagccacagcttaccccaagttcaccgagagggctgtcgtagacgtgccgatgcccaagaagctctttggttctcagctgaaaacaaaggaccatttcttggaggtgaagatggagagttccaagcagcacttcttccacctctggaacgacttcgcttacattgaagtggatgggaagaagtaccccagttcggaggatggcatccaggtggtggtgattgacgggaaccaagggcgcgtggtgagccacacgagcttcaggaactccattctgcaaggcataccatggcagcttttcaactatgtggcgaccatccctgacaattccatagtgcttatggcatcaaagggaagatacgtctccagaggcccatggaccagagtgctggaaaagcttggggcagacaggggtctcaagttgaaagagcaaatggcattcgttggcttcaaaggcagcttccggcccatctgggtgacactggacactgaggatcacaaagccaaaatcttccaagttgtgcccatccctgtggtgaagaagaagaagttgtgaggacagctgccgcccggtgccacctcgtggtagactatg

SEQ ID NO: 5 - DNA, 25 bp, Homo sapiens
caaagtggcagggaaaccaatgtac

SEQ ID NO: 6 - DNA, 73 bp, Homo sapiens
gtgcttccctgcagatcctccgcccccaaccaggtcaaagtggcagggaaaccaatgtacctgcacatcgggg

SEQ ID NO: 7 - DNA, 25 bp, Homo sapiens
acccagagggagcacactgccagga

SEQ ID NO: 8 - DNA, 59 bp, Homo sapiens
gtgacactgtctcggctacagacccagagggagcacactgccaggatgggagctgctgg

SEQ ID NO: 9 - DNA, 25 bp, Homo sapiens
gacggccacgcagtacttcatcctc

SEQ ID NO: 10 - DNA, 25 bp, Homo sapiens
tataacctgggctcggtggaagccc

SEQ ID NO: 11 - DNA, 25 bp, Homo sapiens
agcgcacgagacagccttcctctcc

SEQ ID NO: 12 - DNA, 2252 bp, Homo sapiens
tcggccggagggaggagcccgagccgccgccgccgccgccgcccgcccgcccggcccgggaggaggaccggaccccgagcggctgggagcgcacgcgagcgggccgtggccgcgcgggcagcgccctgagcctgtcccgcgcccgcgggcccggccgagcaggagcagctcccggggatgcccgggcggctcggagcgcgggcagcggcagccgtgcggtgagggcggtggcgcccgcgggacctgcgactgccaggaactcctgccaccgccaccggctcccatggcccacatacccagtgggggtgccccagcagcgggggcagcccccatgggcccccagtattgcgtgtgcaaggtggagctgtcagtgagtggccagaacctactggaccgggatgttacctccaagtccgaccccttctgtgtcctctttacagagaacaatggcagatggatcgagtacgacaggacagaaaccgcgatcaacaacctcaaccccgccttctccaagaagttcgtgcttgactaccacttcgaggaggtacagaagctcaagttcgcgctctttgaccaggacaagtccagtatgcggctggacgagcatgacttcctgggccagttctcctgcagcctgggcacgatcgtctccagcaagaagatcactaggcctctgctgctgctgaatgacaagcctgcggggaagggcttgattacgatcgctgcccaggagctgtccgacaaccgcgtcatcacactaagcctggcgggcaggaggctggacaagaaggacctctttgggaagtcagacccctttctggagttttataagccaggagacgatggcaagtggatgctggtccacaggactgaggtgatcaagtacacactggaccctgtgtggaagccattcacagtgcccttggtgtccctgtgtgatggggacatggagaagcccatccaggtcatgtgctacgactatgacaatgacgggggccatgacttcatcggcgagttccagacctcagtgtcacagatgtgtgaggctcgagacagcgtcccgctggagttcgagtgcatcaaccccaagaagcagaggaagaagaagaactataaaaactcgggcatcatcatcctgcgatcctgcaagataaaccgagactactccttccttgactacatcctgggaggctgccagctcatgttcaccgttggaatagactttacagcctccaacgggaatcccctcgacccttcctctttgcactatatcaaccctatgggcaccaacgaatatctgtcggccatctgggctgttgggcagatcattcaggactacgacagtgataagatgtttccagctctgggattcggggcccagttacccccagactggaaggtctcccatgagtttgccatcaacttcaaccccaccaaccccttctgctcaggtgtggatggtattgcccaggcgtactcagcttgcctgccccacatccgcttctacggtcctaccaatttctcccccatcgtcaaccacgtggcccggtttgcggcccaggccacacaacagcggacggccacgcagtacttcatcctcctcatcatcacggacggggtcatcagtgacatggaggagacacggcatgccgtggtgcaggcttccaagctgcccatgtccatcatcatcgtgggcgtgggcaatgcggacttcgctgccatggagttcctggatggggacagccgcatgctgcgctcccacacgggggaggaggcagcccgcgatattgtgcagttcgttccctttcgagagttccgcaacgcagcaaaagagaccttggccaaagctgtgctggcggagctgccccaacaagttgtgcagtatttcaagcataaaaacctgccccccaccaactcggagcccgcctgagctccagtgcccagcagcagcatgtcagctgagcctcctgccctcccccaggaacatgcacgctcactctgcttccttgtgggtggcctttttttaccgatccccttttttattttttacaaccggacctccacccccaacttcctccagcccagctgggcttcctttgttggagtcaactgttgatgcttccaggccaaactggcttcctctcctcctctccccacctttgccattcttaagtattgaatgtactttgtataattttagtggaattgttattgagaataaaatttttacaatcataaaaaaaaaaaaaaaa

SEQ ID NO: 13 - DNA, 1583 bp, Homo sapiens
acaaagcggttggttgcggcagggactacaccggtgtatcatgggagcggcccgggctacacgttaaaaacactaaggggagcgcgcgaagctgaacttggcgctcgatgggggccgttagccgccctagagcgcgcggagccgcagaggcgtagctggactacaacgcagtgcatctcgggaggccaactcgactggactgggtgagaggacagaggtggctcgatgggcggcccgaaggccggggatcatggcgggaaggcgggcccagacaggttcagccccgccccgacccgccgctccccaccccccggccggcctcgcgtgccttcccgcagcactgccgtccccgggatgctgagcgcccaccgtctccccgcagccccctcatgcccggctgcgagctgcccgtgggcacctgcccggacatgtgcccggccgccgagcgcgcccagcgcgaaagggagcaccgcctgcaccgcttggaggtggtgccgggttgccgccaggacccgccccgcgcggatccgcagcgcgcggtgaaggagtacagccgacccgccgccggcaagccccggcccccgcccagccagttgcgtccgccctccgtgctgctggccaccgtgcgctacctggccggtgaggtggcggagagcgccgacatcgcccgcgccgaggtggccagcttcgtggcagaccgcttgcgagctgtgctcctggacctggcgctgcagggagcgggcgacgccgaggcagctgtggtgctggaggcggcgctggccacgctgctgaccgtagtggcgcggctcgggcccgacgcggcgcggggacccgcggacccggtgctgctgcaggcccaggtgcaggagggcttcggctcgctgcggcgctgctacgcgcggggcgccgggccgcacccccgccaacccgccttccagggcctctttctgctctataacctgggctcggtggaagccctgcatgaggttctacagctgcctgctgccctgcgcgcctgcccgcccctccgcaaggccttggcggtagatgctgccttccgagagggcaatgctgcccgcctgttccgtctgctccagaccctgccctacctgccaagttgcgctgtgcagtgccatgtgggccatgcccgccgggaagccctggcccgcttcgctcgtgcctttagcacccccaagggccagaccttgcctctgggcttcatggtcaacctcttggccctggatggactcagggaagcacgggacctgtgccaggcccacgggctgcccttggacggagaggagagagttgtgttcctgaggggtcgctacgtggaggaagggctaccgcctgccagtacgtgcaaggtgttagtggagagcaaacttcgaggacgtaccctggaggaggtggtcatggcagaggaggaagatgagggcacggacagacctgggtccccagcctgaggagggagcgtgagcctcccagagccccaggactgggccagagcacttaggtttctttttccatggtttccaggtaataaaaggaacttgttttgttggtaaaaaaaaaaaaaaaaa

SEQ ID NO: 14 - DNA, 696 bp, Homo sapiens
agtgcgaatcatgggaggcggctggtggtgggctcgggccgctcgccttgcccgtcttcgcttccggaggtcgctactgccgcctcagcggccccggagcgggggcgcccgggggtccttcgcccccggccacggtccccgcgccggggcttcgccgcccccagtgtccgagctggatcgtgcggacgcctggctcctccgaaaagcgcacgagacagccttcctctcctggttccgcaatggcctcctggcatcgggcatcggggtcatctccttcatgcagagtgacatgggtcgggaagcagcatatggcttcttcctgctgggcggcctgtgcgtggtgtggggcagcgcctcgtacgccgtgggcctggcggcgctgcgaggacccatgcagctgacgctggggggcgcggccgtgggcgcgggcgccgtgctggccgccagcctgctctgggcgtgcgccgtgggcctctacatggggcagctggagctggacgtggagctggtgcccgaggacgacgggacggcctccgcggaaggccctgatgaggcgggtcggccgccacccgagtgagcgacacggccgtggggcctggcaggcgctggacagcgcccgaggactgggacattaaacctgacctcccctcctccaaaaaaaaaaaaaaaaaaaaaaaaaaaaaaaaaaaaaaaaaa

SEQ ID NO: 15 - protein, 6 aa, Artificial Sequence
  Description of Artificial Sequence Synthetic 6xHis tag
HHHHHH

SEQ ID NO: 16 - DNA, 25 bp, Artificial Sequence
  Description of Artificial Sequence Synthetic primer
aggcgtgacactgtctcggctacag

SEQ ID NO: 17 - DNA, 22 bp, Artificial Sequence
  Description of Artificial Sequence Synthetic primer
ccactccacgtcttgaacccac

SEQ ID NO: 18 - DNA, 26 bp, Artificial Sequence
  Description of Artificial Sequence Synthetic primer
gacctctccatccatcatacattctc

SEQ ID NO: 19 - DNA, 23 bp, Artificial Sequence
  Description of Artificial Sequence Synthetic primer
ccagccagttgtcattcttcgtg

SEQ ID NO: 20 - DNA, 23 bp, Artificial Sequence
  Description of Artificial Sequence Synthetic primer
cccaggttattcagagcacattc

SEQ ID NO: 21 - DNA, 23 bp, Artificial Sequence
  Description of Artificial Sequence Synthetic primer
tggcagagatgattgagaggaac

SEQ ID NO: 22 - DNA, 24 bp, Artificial Sequence
  Description of Artificial Sequence Synthetic primer
cgtgacactgtctcggctacagac

SEQ ID NO: 23 - DNA, 23 bp, Artificial Sequence
  Description of Artificial Sequence Synthetic primer
caacttcttcttcttcaccacag

SEQ ID NO: 24 - DNA, 59 bp, Artificial Sequence
  Description of Artificial Sequence Synthetic primer
tcaacccatttgctgtccaccagtcatgctagccatcaacttcttcttcttcaccacag

SEQ ID NO: 25 - DNA, 4505 bp, Homo sapiens
gagctagcgctcaagcagagcccagcgcggtgctatcggacagagcctggcgagcgcaagcggcgcggggagccagcggggctgagcgcggccagggtctgaacccagatttcccagactagctaccactccgcttgcccacgccccgggagctcgcggcgcctggcggtcagcgaccagacgtccggggccgctgcgctcctggcccgcgaggcgtgacactgtctcggctacagacccagagagaaaagcttcattctggaggggaaggagttttgagtgccaaggatgaaattccacccatcactcggtctctgagctgcaggacacaggcaggacaacggtaggattttcatgccccgatctgcctggccttgagttgtggcagctggggaagccactgggagcacactgccaggatgggagctgctgggaggcaggacttcctcttcaaggccatgctgaccatcagctggctcactctgacctgcttccctggggccacatccacagtggctgctgggtgccctgaccagagccctgagttgcaaccctggaaccctggccatgaccaagaccaccatgtgcatatcggccagggcaagacactgctgctcacctcttctgccacggtctattccatccacatctcagagggaggcaagctggtcattaaagaccacgacgagccgattgttttgcgaacccggcacatcctgattgacaacggaggagagctgcatgctgggagtgccctctgccctttccagggcaatttcaccatcattttgtatggaagggctgatgaaggtattcagccggatccttactatggtctgaagtacattggggttggtaaaggaggcgctcttgagttgcatggacagaaaaagctctcctggacatttctgaacaagacccttcacccaggtggcatggcagaaggaggctatttttttgaaaggagctggggccaccgtggagttattgttcatgtcatcgaccccaaatcaggcacagtcatccattctgaccggtttgacacctatagatccaagaaagagagtgaacgtctggtccagtatttgaacgcggtgcccgatggcaggatcctttctgttgcagtgaatgatgaaggttctcgaaatctggatgacatggccaggaaggcgatgaccaaattgggaagcaaacacttcctgcaccttggatttagacacccttggagttttctaactgtgaaaggaaatccatcatcttcagtggaagaccatattgaatatcatggacatcgaggctctgctgctgcccgggtattcaaattgttccagacagagcatggcgaatatttcaatgtttctttgtccagtgagtgggttcaagacgtggagtggacggagtggttcgatcatgataaagtatctcagactaaaggtggggagaaaatttcagacctctggaaagctcacccaggaaaaatatgcaatcgtcccattgatatacaggccactacaatggatggagttaacctcagcaccgaggttgtctacaaaaaaggccaggattataggtttgcttgctacgaccggggcagagcctgccggagctaccgtgtacggttcctctgtgggaagcctgtgaggcccaaactcacagtcaccattgacaccaatgtgaacagcaccattctgaacttggaggataatgtacagtcatggaaacctggagataccctggtcattgccagtactgattactccatgtaccaggcagaagagttccaggtgcttccctgcagatcctgcgcccccaaccaggtcaaagtggcagggaaaccaatgtacctgcacatcggggaggagatagacggcgtggacatgcgggcggaggttgggcttctgagccggaacatcatagtgatgggggagatggaggacaaatgctacccctacagaaaccacatctgcaatttctttgacttcgatacctttgggggccacatcaagtttgctctgggatttaaggcagcacacttggagggcacggagctgaagcatatgggacagcagctggtgggtcagtacccgattcacttccacctggccggtgatgtagacgaaaggggaggttatgacccacccacatacatcagggacctctccatccatcatacattctctcgctgcgtcacagtccatggctccaatggcttgttgatcaaggacgttgtgggctataactctttgggccactgcttcttcacggaagatgggccggaggaacgcaacacttttgaccactgtcttggcctccttgtcaagtctggaaccctcctcccctcggaccgtgacagcaagatgtgcaagatgatcacagaggactcctacccagggtacatccccaagcccaggcaagactgcaatgctgtgtccaccttctggatggccaatcccaacaacaacctcatcaactgtgccgctgcaggatctgaggaaactggattttggtttatttttcaccacgtaccaacgggcccctccgtgggaatgtactccccaggttattcagagcacattccactgggaaaattctataacaaccgagcacattccaactaccgggctggcatgatcatagacaacggagtcaaaaccaccgaggcctctgccaaggacaagcggccgttcctctcaatcatctctgccagatacagccctcaccaggacgccgacccgctgaagccccgggagccggccatcatcagacacttcattgcctacaagaaccaggaccacggggcctggctgcgcggcggggatgtgtggctggacagctgccggtttgctgacaatggcattggcctgaccctggccagtggtggaaccttcccgtatgacgacggctccaagcaagagataaagaacagcttgtttgttggcgagagtggcaacgtggggacggaaatgatggacaataggatctggggccctggcggcttggaccatagcggaaggaccctccctataggccagaattttccaattagaggaattcagttatatgatggccccatcaacatccaaaactgcactttccgaaagtttgtggccctggagggccggcacaccagcgccctggccttccgcctgaataatgcctggcagagctgcccccataacaacgtgaccggcattgcctttgaggacgttccgattacttccagagtgttcttcggagagcctgggccctggttcaaccagctggacatggatggggataagacatctgtgttccatgacgtcgacggctccgtgtccgagtaccctggctcctacctcacgaagaatgacaactggctggtccggcacccagactgcatcaatgttcccgactggagaggggccatttgcagtgggtgctatgcacagatgtacattcaagcctacaagaccagtaacctgcgaatgaagatcatcaagaatgacttccccagccaccctctttacctggagggggcgctcaccaggagcacccattaccagcaataccaaccggttgtcaccctgcagaagggctacaccatccactgggaccagacggcccccgccgaactcgccatctggctcatcaacttcaacaagggcgactggatccgagtggggctctgctacccgcgaggcaccacattctccatcctctcggatgttcacaatcgcctgctgaagcaaacgtccaagacgggcgtcttcgtgaggaccttgcagatggacaaagtggagcagagctaccctggcaggagccactactactgggacgaggactcagggctgttgttcctgaagctgaaagctcagaacgagagagagaagtttgctttctgctccatgaaaggctgtgagaggataaagattaaagctctgattccaaagaacgcaggcgtcagtgactgcacagccacagcttaccccaagttcaccgagagggctgtcgtagacgtgccgatgcccaagaagctctttggttctcagctgaaaacaaaggaccatttcttggaggtgaagatggagagttccaagcagcacttcttccacctctggaacgacttcgcttacattgaagtggatgggaagaagtaccccagttcggaggatggcatccaggtggtggtgattgacgggaaccaagggcgcgtggtgagccacacgagcttcaggaactccattctgcaaggcataccatggcagcttttcaactatgtggcgaccatccctgacaattccatagtgcttatggcatcaaagggaagatacgtctccagaggcccatggaccagagtgctggaaaagcttggggcagacaggggtctcaagttgaaagagcaaatggcattcgttggcttcaaaggcagcttccggcccatctgggtgacactggacactgaggatcacaaagccaaaatcttccaagttgtgcccatccctgtggtgaagaagaagaagttgtga

(19) United States
(12) Patent Application Publication
(10) Pub. No.: US 2015/0167098 A1
(43) Pub. Date: Jun. 18, 2015

(54) METHODS FOR CHARACTERIZING PATIENTS WITH COLON CANCER

(71) Applicant: Case Western Reserve University, Cleveland, OH (US)

(72) Inventors: Sanford D. Markowitz, Pepper Pike, OH (US); Stephen Fink, University Heights, OH (US); Lois Myeroff, Chardon, OH (US)

(21) Appl. No.: 14/415,557

(22) PCT Filed: Jul. 17, 2013
(86) PCT No.: PCT/US13/50873

§ 371 Date: Jan. 16, 2015

Related U.S. Application Data
(60) Provisional application No. 61/673,051, filed on Jul. 18, 2012.
(60) Provisional application No. 61/738,934, filed on Dec. 18, 2012.

(51) Int. Cl. C12Q 1/68

(52) U.S. Cl. CPC C12Q 1/6886; C12Q 2600/118; C12Q 2600/158

(57) ABSTRACT

The present application provides methods for characterizing colon cancer patients by measuring their ColoUp-1 expression levels. In particular, the present application provides among other things methods for determining the survival prognosis of colon cancer patients and methods for determining the efficacy of treatment regimens of colon cancer patients.

### RELATED APPLICATIONS

This application claims the benefit of priority to U.S. provisional application No. 61/673,051, filed Jul. 18, 2012 and U.S. provisional application No. 61/738,934, filed Dec. 18, 2012. The disclosures of the foregoing applications are hereby incorporated by reference in their entirety.

### FUNDING

Work described herein was funded, in part, by grant number NIH RO1 CA120237, UO1 CA152756 and P50 CA150964. The United States government has certain rights in the invention.

### BACKGROUND

Colorectal cancer, also referred to herein as colon cancer, is the second leading cause of cancer mortality in the adult American population. An estimated 135,000 new cases of colon cancer occur each year. Although many people die of colon cancer, early stage colon cancers are often treatable by surgical removal (resection) of the affected tissue. Surgical treatment can be combined with chemotherapeutic agents to achieve an even higher survival rate in certain colon cancers. However, the survival rate drops to 5% or less over five years in patients with metastatic (late stage) colon cancer.

Clinical management of colon cancer can be aided by prognosis molecular markers and by therapy predictive molecular markers for chemotherapy and radiation therapy. Prognosis molecular markers assess risk of disease progression independent of therapy. Therapy predictive molecular markers may indicate sensitivity or resistance of a cancer to a specific treatment. For most cancers and cancer treatments, there exist subsets of patients that will respond to a particular treatment and subsets of patients that will fail to respond to the treatment.

An ideal molecular marker for colon cancer should have the quality of being able to identify the patients at an early stage and help the physician select the right therapy for the patient. The molecular marker could also be useful to help surgeons select the right patients for operation. Moreover, molecular markers are also useful for monitoring the disease recurrence/progression after operation or therapy and help the physician evaluate the effect of therapy in order to give a more patient-specific treatment. The use of predictive molecular markers to identify subsets of patients likely to respond to a particular therapy would facilitate selection of appropriate treatment and avoid unnecessary delays associated with ineffective treatment.

### BRIEF SUMMARY

In some aspects, this application provides a molecular marker that is useful for determining the survival prognosis of a patient afflicted with colon cancer. In particular, the application provides a method of determining colon cancer survival prognosis. In some embodiments, the method involves measuring the ColoUp-1 expression level in a biological sample obtained from a colon cancer patient; and comparing said ColoUp-1 expression level to a median ColoUp-1 expression level in a population of colon cancer patients; wherein a ColoUp-1 expression level above the median ColoUp-1 expression level is indicative of poor survival prognosis, and wherein a ColoUp-1 expression level below the median ColoUp-1 expression level is indicative of good survival prognosis.

In some embodiments, poor survival prognosis comprises a survival time of less than 60 months, less than 50 months, less than 40 months, less than 35 months, or less than 30 months. In some embodiments, good survival prognosis comprises a survival time of more than 60 months, more than 70 months, more than 80 months, more than 90 months, or more than 100 months.

In other aspects, this application provides a molecular marker that is useful for determining the efficacy of a treatment regimen for a colon cancer patient. In particular, the application provides a method of determining whether a treatment regimen is likely to be effective for a colon cancer patient. In some embodiments, the method involves measuring the ColoUp-1 expression level in a biological sample obtained from a colon cancer patient; and comparing said ColoUp-1 expression level to a median ColoUp-1 expression level in a population of colon cancer patients; wherein a ColoUp-1 expression level above the median ColoUp-1 expression level is indicative of the treatment regimen being less likely to be effective in treating the patient, and wherein a ColoUp-1 expression level below the median ColoUp-1 expression level is indicative of the treatment regimen being more likely to be effective in treating the patient.

In some aspects, this application provides a method of determining whether a treatment regimen is likely to be effective for a colon cancer patient comprising: measuring the ColoUp-1 expression level in a biological sample obtained from a colon cancer patient; and comparing said ColoUp-1 expression level to a median ColoUp-1 expression level in a population of colon cancer patients; wherein a ColoUp-1 expression level below the median ColoUp-1 expression level is indicative of the treatment regimen being less likely to be effective in treating the patient, and wherein a ColoUp-1 expression level above the median ColoUp-1 expression level is indicative of the treatment regimen being more likely to be effective in treating the patient.

In other aspects, this application provides a method for treating a colon cancer patient. In some embodiments, the method involves providing a treatment regimen to the patient; measuring the ColoUp-1 expression level in a biological sample obtained from the patient receiving a treatment regimen; comparing said ColoUp-1 expression level to a median ColoUp-1 expression level in a population of colon cancer patients; and modifying the treatment regimen when the ColoUp-1 expression level in the biological sample obtained from the patient is above the median ColoUp-1 expression level. In other embodiments, the method involves providing a treatment regimen to the patient; measuring the ColoUp-1 expression level in a biological sample obtained from the patient receiving a treatment regimen; comparing said ColoUp-1 expression level to a median ColoUp-1 expression level in a population of colon cancer patients; and modifying the treatment regimen when the ColoUp-1 expression level in the biological sample obtained from the patient is below the median ColoUp-1 expression level. In some embodiments, the treatment regimen is modified when the ColoUp-1 expression level in the biological sample obtained from the patient is above the 20^{th}, 30^{th}, or 40^{th} percentile of ColoUp-1 expression levels. In other embodiments, the treatment regimen is modified when the ColoUp-1 expression level in the biological sample obtained from the patient is below the 20^{th}, 30^{th}, or 40^{th} percentile of ColoUp-1 expression levels. In some embodiments, the treatment regimen comprises the administration to the patient of a compound that antagonizes ColoUp-1 function, such as an anti-ColoUp-1 antibody or fragment thereof. In some embodiments, the modification of the treatment regimen comprises increasing the amount or level of treatments already administered to the patient in said treatment regimen. In some embodiments, the modification of the treatment regimen comprises increasing the dosage of chemotherapy or the frequency of dosages of chemotherapy. In some embodiments, the modification of the treatment regimen comprises decreasing the dosage of chemotherapy or decreasing the frequency of dosages of chemotherapy. In some embodiments, the modification of the treatment regimen comprises decreasing the amount of radiation therapy or decreasing the frequency of radiation therapy sessions. In some embodiments, the modification of the treatment regimen comprises discontinuing chemotherapy or radiation therapy. In some embodiments, the modification of the treatment regimen comprises increasing the amount of radiation therapy or the frequency of administration of radiation therapy. In some embodiments, the modification of the treatment regimen comprises switching from one type of treatment regimen to another type of treatment regimen. In some embodiments, the modification of the treatment regimen comprises utilizing a form of treatment for the patient in addition to the treatment regimen. In some embodiments, the modification of the treatment regimen comprises the utilization of resection or anastomosis. In some embodiments, the modification of the treatment regimen comprises the administration to the patient of fluorouracil, bevacizumab, irinotecan hydrochloride, capecitabine, cetuximab, oxaliplatin, leucovorin calcium, panitumumab, regorafenib, ziv-aflibercept, or any combination thereof. In some embodiments, the modification of the treatment regimen comprises the administration to the patient of a compound that antagonizes ColoUp-1 function. In some embodiments, the modification of the treatment regimen comprises administering the compound that antagonizes ColoUp-1 function is an anti-ColoUp-1 antibody or a fragment thereof.

In certain embodiments, the population of colon cancer patients includes patients afflicted with Stage II-IV colon cancer.

In certain embodiments, the population of colon cancer patients includes patients afflicted with Stage I-IV colon cancer.

In certain embodiments, the treatment regimen comprises chemotherapy, radiation therapy, or a combination of chemotherapy and radiation therapy

In some aspects, the biological sample obtained from the colon cancer patient is a tumor sample. In other aspects, the biological sample is a stool sample. In other aspects, the biological sample is blood, including blood fractions such as serum or plasma. For instance, the blood sample obtained from a patient may be further processed such as by fractionation to obtain blood serum or blood plasma. In other aspects, the sample is a urine sample.

In some embodiments, the biological sample is obtained from the patient prior to the start of the treatment regimen. In some embodiments, the biological sample is obtained from the patient 1 month after the start of the treatment regimen. In other embodiments, the biological sample is obtained 2 months after the start of the treatment regimen. In other embodiments, the biological sample is obtained from the patient 3 months after the start of the treatment regimen. In other embodiments, the biological sample is obtained from the patient 4 months after the start of the treatment regimen.

In some embodiments, the method involves measuring ColoUp-1 mRNA levels. In some embodiments, the ColoUp-1 mRNA comprises a nucleic acid sequence that is at least 95%, 98%, 99% or 100% identical to the nucleic acid sequence of SEQ ID No: 4. In other embodiments, the ColoUp-1 mRNA comprises a nucleic acid sequence encoding a polypeptide comprising an amino acid sequence that is at least 95% identical to the amino acid sequence as set forth in any one of SEQ ID No: 1, SEQ ID No: 2 and SEQ ID No: 3. In some embodiments, ColoUp-1 mRNA level is measured by quantitative reverse transcription polymerase chain reaction (qRT-PCR). In other embodiments, ColoUp-1 mRNA level is measured by microarray analysis.

The methods of the present disclosure involve comparing the ColoUp-1 expression level in a colon cancer patient to a median ColoUp-1 expression in a population of colon cancer patients. In embodiments wherein ColoUp-1 expression level is measured by measuring ColoUp-1 mRNA level by microarray, the median ColoUp-1 mRNA expression level is in the range of 220 to 800. In some embodiments, the median ColoUp-1 mRNA expression level is in the range of 250 to 800. In other embodiments, the median ColoUp-1 mRNA expression level is in the range of 250 to 500. In yet other embodiments, the median ColoUp-1 mRNA expression level is in the range of 300 to 500. In yet other embodiments, the median ColoUp-1 mRNA expression level is in the range of 325 to 400. In yet other embodiments, the median ColoUp-1 mRNA expression level is in the range of 350 to 375. In yet other embodiments, the median ColoUp-1 mRNA expression level is 330.

In embodiments wherein ColoUp-1 expression level is measured by measuring ColoUp-1 mRNA level by real-time PCR, the median ColoUp-1 mRNA expression level is in the range of 1.000-1.070, 1.010-1.060, 1.020-1.050 or 1.024 to 1.047. In some embodiments the median value of expression is 0.6-1.5, 0.8-1.2, or 0.9-1.1. In some embodiments of any of the methods disclosed herein, ColoUp-1 mRNA levels are measured by amplifying a nucleic acid sequence comprising the nucleotide sequences of any one of SEQ ID NOs: 5-8. In particular embodiments, ColoUp-1 mRNA levels are measured by amplifying a nucleic acid sequence comprising the nucleotide sequences of SEQ ID NOs: 7 or 8. In some embodiments, the ColoUp-1 expression level in a patient's biological sample is normalized against the expression levels of one or more reference transcripts. In some embodiments, the reference transcript is selected from the group comprising: DDA1, SAC3D1, TMEM160, CPNE2, TMEM134, ZNF787, ZNF746, or SIRT3. In particular embodiments, the reference transcript is selected from the group comprising: CPNE2, SAC3D1, or TMEM160. In some embodiments, at least one of the reference transcripts is CPNE2, and levels of CPNE2 are measured by amplifying an mRNA product comprising the sequence of SEQ ID NO: 9. In some embodiments, at least one of the primers used to amplify CPNE2 binds the sequence of SEQ ID NO: 9. In some embodiments, at least one of the primers or probes used to detect CPNE2 binds the sequence of SEQ ID NO: 9. In some embodiments, the CPNE2 mRNA product spans the exon 14/15 boundary of the CPNE2 gene. In some embodiments, the CPNE2 mRNA product is 50-100 base pairs in length and has a sequence midpoint corresponding to base pair 1588 of SEQ ID NO: 12. In some embodiments, at least one of the reference transcripts is SAC3D1, and levels of SAC3D1 are measured by amplifying an mRNA product comprising the sequence of SEQ ID NO: 10. In some embodiments, at least one of the primers used to amplify SAC3D1 binds the sequence of SEQ ID NO: 10. In some embodiments, at least one of the primers or probes used to detect SAC3D1 binds the sequence of SEQ ID NO: 10. In some embodiments, the SAC3D1 mRNA product spans the exon 1/2 boundary of the SAC3D1 gene. In some embodiments, the SAC3D1 mRNA product is 50-100 base pairs in length and has a sequence midpoint corresponding to base pair 966 of SEQ ID NO: 13. In some embodiments, at least one of the reference transcripts is TMEM160, and levels of TMEM160 are measured by amplifying an mRNA product comprising the sequence of SEQ ID NO: 11. In some embodiments, at least one of the primers used to amplify TMEM160 binds the sequence of SEQ ID NO: 11. In some embodiments, at least one of the primers or probes used to detect TMEM160 binds the sequence of SEQ ID NO: 11. In some embodiments, the TMEM160 mRNA product spans the exon 1/2 boundary of the TMEM160 gene. In some embodiments, the TMEM160 mRNA product is 50-100 base pairs in length and has a sequence midpoint corresponding to base pair 217 of SEQ ID NO: 14. In some embodiments, the normalizing step comprises normalizing against the geometric mean of the expression levels of the one or more reference transcripts. In some embodiments, the normalizing step comprises normalizing against the geometric mean of the Cq values of the reference transcripts. In some embodiments, the normalizing step comprises normalizing against the geometric mean of the Ct values of the reference transcripts. In some embodiments, the normalizing step comprises normalizing against the arithmetic mean of the expression levels of the reference transcripts. In some embodiments, the normalizing step comprises normalizing against the arithmetic mean of the Cq values of the reference transcripts. In some embodiments, the normalizing step comprises normalizing against the arithmetic mean of the Ct values of the reference transcripts.

ColoUp-1 expression level may also be measured by measuring ColoUp-1 protein expression level. In some embodiments, the ColoUp1 protein is encoded by a nucleic acid sequence that is at least 95%, 98%, 99% or 100% identical to the nucleic acid sequence of SEQ ID No: 4. In other embodiments, the ColoUp1 protein comprises an amino acid sequence that is at least 95% identical to the amino acid sequence as set forth in any one of SEQ ID No: 1, SEQ ID No: 2 and SEQ ID No: 3. In some embodiments, the ColoUp-1 protein level is measured by an assay that employs an antibody. In some embodiments, the antibody detects a 150 kDa ColoUp-1 protein. In other embodiments, the antibody detects a 100 kDa ColoUp-1 protein. In some embodiments, the assay that employs an antibody is an enzyme-linked immunosorbent assay (ELISA), quantitative immunohistochemical analysis, western blotting, or flow cytometry.

In other aspects, this application provides a method for treating colon cancer in a patient having a ColoUp-1 expression level above a median ColoUp-1 expression level in a population of colon cancer patients, comprising administering a therapeutically effective amount of a cancer therapeutic to the patient. In other aspects, this application provides a method for treating colon cancer in a patient having a ColoUp-1 expression level below a median ColoUp-1 expression level in a population of colon cancer patients, comprising administering a therapeutically effective amount of a cancer therapeutic to the patient.

In some embodiments, the method of prognosis comprises the steps of measuring the ColoUp-1 expression level in a biological sample obtained from a colon cancer patient; and comparing said ColoUp-1 expression level in the biological sample to a ColoUp-1 expression level in a population of colon cancer patients; wherein a ColoUp-1 expression level in the biological sample greater than the ColoUp-1 expression level in the 25^{th}, 30^{th}, 40^{th}, 50^{th}, 60^{th}, 70^{th}, 80^{th}, 90^{th} or 95^{th} percentile of the population of colon cancer patients is indicative of poor survival prognosis. In some embodiments, the method of prognosis comprises the steps of measuring the ColoUp-1 expression level in a biological sample obtained from a colon cancer patient; and comparing said ColoUp-1 expression level in the biological sample to a ColoUp-1 expression level in a population of colon cancer patients; wherein a ColoUp-1 expression level in the biological sample greater than a ColoUp-1 expression level of the 25^{th}-75^{th}, 30^{th}-70^{th}, or 40^{th} to 60^{th} percentile of the population of colon cancer patients is indicative of poor survival prognosis.

In some embodiments, the method is a method of determining a Stage II or a Stage III colon cancer prognosis comprising: measuring the ColoUp-1 expression level in a biological sample obtained from a colon cancer patient; and comparing said ColoUp-1 expression level in the biological sample to a ColoUp-1 expression level in a population of colon cancer patients; wherein a ColoUp-1 expression level in the biological sample greater than the ColoUp-1 expression level of the 25^{th}, 40^{th}, 50^{th}, 60^{th} or 70^{th} percentile of the population of colon cancer patients is indicative of poor survival prognosis. In some embodiments, a ColoUp-1 expression level in the biological sample greater than the ColoUp-1 expression level of the 25^{th}-75^{th}, 30^{th}-70^{th}, or 40^{th}-60^{th} percentile of the population of colon cancer patients is indicative of poor survival prognosis. In some embodiments, the population of colon cancer patients is a population of colon cancer patients with Stage III colon cancer.

The embodiments and practices of the present invention, other embodiments, and their features and characteristics, will be apparent from the description, figures and claims that follow, with all of the claims hereby being incorporated by this reference into this Summary.

### BRIEF DESCRIPTION OF THE DRAWINGS

FIG. 1. KIAA1199/ColoUp-1 mRNA expression in normal colon epithelium and colon cancer samples. (A) Expression levels of KIAA1199 measured on GeneChip microarrays for samples of normal colon epithelium, colon adenomas, colon cancer primary tumors of stages II, III and IV, colon cancer hepatic metastases, and colon cancer cell lines. Horizontal bars denote median expression values within each group. Transcript hybridization to expression microarrays is measured in Average Intensity units (AIU). (B) Northern blot analysis of ColoUp-1 expression in 6 normal colon epithelium samples versus colon cancer cell lines (upper panel). (C) Northern blot analysis of ColoUp-1 expression in 15 samples of colon cancer tissue (T) and paired normal colonic mucosa (N). The lower panels in both B and C are the ethidium bromide stainings of the 28S ribosomal RNA subunit for each of the corresponding samples. Northern hybridization probe used spanned ColoUp-1 cDNA sequences in exons 1-9. (D) Real-time PCR measurement of ColoUp-1 transcript expression. Shown is the ratio of ColoUp-1 expression in colon cancer versus matched normal colon mucosa as determined in 29 patients. ColoUp-1 values are normalized against expression of the house-keeping gene Beta-2-microglobulin. The mean value is indicated as a horizontal black bar.

FIG. 2. Structure of the ColoUp-1 gene. (A) Black boxes denote ColoUp-1 gene exons comprising the short ColoUp-1 transcript, with the white box denoting the additional exon included in the longer form of the transcript. (B) Nucleotide sequence of the ColoUp-1 coding region, with the start and stop codons in italics and underlined, and the exon in the long form of the transcript in bold. (C) Amino acid sequence specified by the ColoUp-1 coding region.

FIG. 3. Induction of endogenous ColoUp-1 protein in colon cancers. (A) Detection of endogenous ColoUp-1 protein by serial immunoprecipitation and western blot analysis using monoclonal antibody PW-3 on a panel of lysates from colon cancer tumor tissues (T) versus matched normal colonic mucosa (N) from 10 different colon cancer patients. Purified T7 epitope tagged ColoUp-1 protein serves as a positive (+) control. (B) Immunostaining of ColoUp-1 protein expression. Shown is immunostaining of ColoUp-1 protein using anti-ColoUp-1 monoclonal antibody PW-3, in 3 cases of colon cancer tumors versus adjacent normal colonic mucosa.

FIG. 4. Western blot analysis of lysates from ColoUp-1 expressing FET colon cancer cells versus ColoUp-1 non-expressing RKO colon cancer cells using anti-ColoUp-1 monoclonal antibody PW-3.

FIG. 5. Secretion of ColoUp-1 protein. (A) Western blot assay of ColoUp-1 protein in lysates of ColoUp-1 transfected cells (Cell Lysate) versus in the immunoprecipitates from a corresponding amount of cell culture media (Media I.P.). Cells were transfected with expression vectors encoding either V5 epitope tagged ColoUp-1 (ColoUp1-V5) or T7 epitope tagged ColoUp-1 (ColoUp1-T7). Immunoprecipitation and Western blotting were performed using antibodies against the V5 epitope-tag, with ColoUp1-T7 samples serving as a negative control. Results are shown for transient transfections performed in both SW480 and VACO-400 colon cancer cell lines. An arrowhead denotes the position of the ˜150 kDa ColoUp-1 protein detected in both cell culture medium and lysates of ColoUp-1 transfected cells. (B) Detection of endogenous ColoUp-1 secreted from colon cancer cells using serial immunoprecipitation and Western blot analysis. Shown are assays of ColoUp-1 protein from 1 ml of cell culture media from colon cancer cell lines FET and V411 that express ColoUp-1 transcript, versus from cell lines V364 and RKO that are negative for ColoUp-1 transcript expression. Also shown for comparison are assay of a matched cell pellet lysate from the ColoUp-1 expressing FET cells. Samples of FET cells assayed represent 3% of the total FET cell pellet and 2% of the corresponding FET media. Media from ColoUp-1 transfected HeLa clone A11-4 serves as a positive control. (C) Western blot Assay for ColoUp-1 in cell culture media from HeLa cell clone A11-4 bearing a doxycycline inducible ColoUp-1-V5 expression vector and grown in the presence [Dox (+)] and absence [Dox (−)] of doxycycline. Lanes labeled Empty Pool 1 show analysis of control media collected from HeLa cells transfected with an empty expression vector. Also indicated is the volume of cell culture media analyzed in each assay. (D) Assay for ColoUp-1 in plasma harvested from 6 athymic mice bearing xenografts of HeLa cell clone A11-4 cells, and maintained on drinking water supplemented with doxycycline (lanes labeled Clone A11-4). Also shown is an assay of plasma harvested from 8 control mice bearing xenografts from HeLa cells transfected with an empty expression vector (lanes labeled Empty Pool 1).

FIG. 6. Detection of ColoUp-1 in human plasma. Shown is the ColoUp-1 protein level in patient plasma from 20 normal subjects and 17 colon cancer patients, expressed as ng/100 μl of plasma. Horizontal bars within the boxes denote median expression values, while “+” denote mean expression values within each group. Whiskers denote the minimum and maximum ColoUp-1 levels detected for each group.

FIG. 7. Gene knockout of ColoUp-1 in DLD-1 cells. (A) Schematic diagram for targeting exon 2 for deletion in ColoUp-1. (B) RT-PCR confirmation for deletion of exon 2 in ColoUp-1 deleted DLD-1 clones (Clone A and Clone B). The PCR primers span exon 2 and the expected band size for exon 2 deleted cells is 343 bp versus 453 bp for non-targeted DLD-1 cells (ColoUp-1+/+). (C) Western blot for ColoUp-1 deleted clones A and B showing a lack of a 150 kDa band, whereas a band is detected in non-targeted DLD-1 cells (ColoUp-1+/+).

FIG. 8. Growth curves for ColoUp-1 deleted DLD-1 clones (dashed line) as compared to wild-type DLD-1 (solid line) for knock-out Clone A (A), and knock-out Clone B (B). Error bars are standard errors of the mean.

FIG. 9. Reduced tumor growth and increased apoptosis in ColoUp-1 negative tumor xenografts. (A-B) Xenograft growth curves in athymic mice injected with ColoUp-1 knockout DLD-1 cell clone A (dashed line) or wild-type DLD-1 cells (solid line), performed in two separate experiments. (C-D) Xenograft growth curves in athymic mice injected with ColoUp-1 knockout DLD-1 cell clone B (dashed lines) or wild-type DLD-1 cells, performed in two separate experiments. Error bars are standard errors of the mean. (E-F) Shown is immunostaining for cleaved caspase-3 in harvested xenografts from mice injected with wild-type, ColoUp-1 expressing DLD-1 cells (E), or ColoUp-1 knockout DLD-1 cells (F).

FIG. 10. Kaplan-Meier survival curves of colon cancer patients with tumor ColoUp-1 expression levels above (dashed line) or below (solid line) the median for each group shown. (A) Analysis of 121 colon cancer patients of stages II, III, and IV demonstrating decreased survival among those with high versus low tumor ColoUp-1 (P=0.02). (B) Analysis of 41 cases of stage IV colon cancer demonstrating decreased survival among those with high versus low tumor ColoUp-1 (P=0.024).

FIG. 11. Kaplan-Meier analyses of survival in ColoUp-1 high (values greater than 1.024) versus ColoUp-1 low (values less than 1.024) colon cancer cases. (A) Survival curve of 31 stage III colon cancer patients with tumor ColoUp-1 expression levels above (dashed line, n=16) or below (solid line, n=15) the median value of 1.024, demonstrating decreased survival in those with high tumor ColoUp-1 transcript levels (P=0.004). (B) Survival curve of 73 stage II and stage III colon cancer patients with tumor ColoUp-1 expression levels above (dashed line) or below (solid line) 1.024, demonstrating decreased survival in those with high tumor ColoUp-1 transcript levels (P=0.0003).

FIG. 12. Cq values for individual SAC3D1, TMEM160, and CPNE2 transcripts determined in 1 ng of input total RNA across a panel of 28 colon tumor samples. The geometric mean of the Cq values (GEO3) for all three genes is also plotted. Note the y-axis scale begins at Cq=20.

### DETAILED DESCRIPTION

### 1. Definitions:

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term antibody also includes polyclonal, monoclonal, or other purified preparations of antibodies and recombinant antibodies.

As used herein, the phrase “gene expression” or “protein expression” includes any information pertaining to the amount of gene transcript or protein present in a sample, as well as information about the rate at which genes or proteins are produced or are accumulating or being degraded (eg. reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information”. Such information may be given in the form of amounts per cell, amounts relative to a control gene or protein, in unitless measures, etc.; the term “information” is not to be limited to any particular means of representation and is intended to mean any representation that provides relevant information. The term “expression levels” refers to a quantity reflected in or derivable from the gene or protein expression data, whether the data is directed to gene transcript accumulation or protein accumulation or protein synthesis rates, etc.

As used herein, the term “nucleic acid” refers to polynucleotides such as deoxyribonucleic acid (DNA), and, where appropriate, ribonucleic acid (RNA). The term should also be understood to include analogs of either RNA or DNA made from nucleotide analogs, and, as applicable to the embodiment being described, single-stranded (such as sense or antisense) and double-stranded polynucleotides. Moreover, it should be noted that, since the nucleotide sequence represented by, for example, SEQ ID NO:4 indicates both of nucleotide sequences of mRNA and cDNA obtained using the same as a template, when mRNA is referred to, of course, since uracil (u) may be contained in place of thymine (t), “t” can be replaced by “u”. In addition, “measurement” encompasses any concept of quantitative measurement, semi-quantitative measurement and detection. Furthermore, “measurement of mRNA” encompasses, in addition to a case of measuring mRNA directly, a case where after the mRNA is once converted to cDNA, the aforementioned cDNA is measured (RT-PCR and the like as described later), and a case where the mRNA is measured indirectly such as the case when translation product of the mRNA is measured. Moreover, in the following description, mRNA, cDNA, nucleic acid and so on which comprises the nucleotide sequence represented by SEQ ID NO: 4 are sometimes referred to as “mRNA of SEQ ID NO: 4, cDNA of SEQ ID NO: 4, nucleic acid of SEQ ID NO: 4, and so on”, respectively.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. Various alignment algorithms and/or programs may be used, including FASTA, BLAST or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

### 2. Overview:

The colon is a portion of the intestinal tract that is roughly three feet in length, stretching from the end of the small intestine to the rectum. Viewed in cross section, the colon consists of four distinguishable layers arranged in concentric rings surrounding an interior space, termed the lumen, through which digested materials pass. In order, moving outward from the lumen, the layers are termed the mucosa, the submucosa, the muscularis propria and the subserosa. The mucosa includes the epithelial layer (cells adjacent to the lumen), the basement membrane, the lamina propria and the muscularis mucosae. In general, the “wall” of the colon is intended to refer to the submucosa and the layers outside of the submucosa. The “lining” is the mucosa.

Precancerous colon neoplasias are referred to as adenomas or adenomatous polyps. Adenomas are typically small mushroom-like or wart-like growths on the lining of the colon and do not invade into the wall of the colon. Adenomas may be visualized through a device such as a colonoscope or flexible sigmoidoscope. Several studies have shown that patients who undergo screening for and removal of adenomas have a decreased rate of mortality from colon cancer. For this and other reasons, it is generally accepted that adenomas are an obligate precursor for the vast majority of colon cancers.

When a colon neoplasia invades into the basement membrane of the colon, it is considered a colon cancer, as the term “colon cancer” is used herein. In describing colon cancers, this specification will generally follow the so-called “TNM” system. Other staging systems have been devised, and the particular system selected is, for the purposes of this disclosure, unimportant. The characteristics that describe a cancer are of greater significance than the particular term used to describe a recognizable stage. The most widely used staging systems generally use at least one of the following characteristics for staging: the extent of tumor penetration into the colon wall, with greater penetration generally correlating with a more dangerous tumor; the extent of invasion of the tumor through the colon wall and into other neighboring tissues, with greater invasion generally correlating with a more dangerous tumor; the extent of invasion of the tumor into the regional lymph nodes, with greater invasion generally correlating with a more dangerous tumor; and the extent of metastatic invasion into more distant tissues, such as the liver, with greater metastatic invasion generally correlating with a more dangerous disease state.

While staging systems vary with the types of cancer, they generally involve the “TNM” system: “T” indicates the type of tumor, “N” indicates whether the cancer has metastasized to nearby lymph nodes; and “M” indicates whether the cancer has metastasized to other parts of the body. Generally, if a cancer is only detectable in the area of the primary lesion without having spread to any lymph nodes it is called Stage I. If it has spread only to the closest lymph nodes, it is called Stage II. In Stage III, the cancer has generally spread to the lymph nodes in near proximity to the site of the primary lesion. Cancers that have spread to a distant part of the body, such as the liver, bone, brain or other site, are Stage IV, the most advanced stage.

### 3. Prognostic Methods for Colon Cancer

The present application is based, at least in part, on the observation that ColoUp-1 is highly expressed in colon cancer, facilitates tumor growth, and correlates with poor patient prognosis. The ColoUp-1 gene encodes a full-length protein of 1361 amino acids. Signal P V1.1 predicts that human ColoUp-1 protein has an N-terminal signal peptide that is cleaved between either amino acids 30-31 (ATS-TV) or amino acids 33-34 (TVA-AG). Four potential glycosylation sites are identified in ColoUp-1 protein. Further, ColoUp-1 protein is predicted to have multiple serine, threonine, and tyrosine phosphorylation sites for kinases such as protein kinase C, cAMP- and cGMP-dependent protein kinases, casein kinase II, and tyrosine kinases. The ColoUp-1 protein shares limited sequence homology to a human transmembrane protein 2 (See Scott et al. 2000 Gene 246:265-74). A mouse ColoUp-1 homolog is identified in existing GenBank databases and is linked with mesoderm development (see Wines et al. 2001 Genomics. 88-98; GenBank entry AAG41062, AY007815 for the 1179 bp nucleic acid sequence entry, with 363/390 (93%) identities with human ColoUp1). ColoUp-1 is also known as CCSP-1 or KIAA1199 and these terms are used interchangeably herein.

Accordingly, the disclosure provides methods for detecting and measuring the levels of molecular markers, in particular ColoUp-1 transcript and protein. In further embodiments, a method of the disclosure comprises providing a biological sample from a subject afflicted with colon cancer, and probing the biological sample to determine the expression level of ColoUp-1. Information regarding the expression level of ColoUp-1 can then be used to draw inferences about the health state of the subject, e.g., survival prognosis and/or responsiveness to colon cancer therapy.

In some aspects, the disclosure provides methods to determine colon cancer prognosis comprising: obtaining a sample from a colon cancer patient and measuring the ColoUp-1 expression level in the biological sample. The methods also include determining a median expression level from a population of colon cancer patients, and subsequently comparing the ColoUp-1 expression level of the patient's biological sample to the median ColoUp-1 expression level obtained from the population of colon cancer patients. In some embodiments, a patient having a ColoUp-1 expression level above the median ColoUp-1 expression level indicates poor survival prognosis. In further embodiments, a patient with a ColoUp-1 expression level below the median ColoUp-1 expression level indicates good survival prognosis.

The efficacy of a treatment regimen can also be inferred based on whether a patient's ColoUp-1 expression level is above or below the median ColoUp-1 expression level of a population of colon cancer patients. Accordingly, the disclosure also provides methods of determining whether a treatment regimen is likely to be effective for a colon cancer patient. In some embodiments, a patient having a ColoUp-1 expression level above the median ColoUp-1 expression level indicates that the treatment regimen is less likely to be effective in treating the patient. In further embodiments, a patient having a ColoUp-1 expression level below the median ColoUp-1 expression level indicates that the treatment regimen is more likely to be effective in treating the patient. Such treatment regimen may consist of surgery, individual therapy, chemotherapy, radiation therapy or any combination thereof.

In certain aspects, the methods of the present disclosure are directed to a method of treating a colon cancer patient. In some embodiments, the method involves providing a treatment regimen to a colon cancer patient, measuring the level of ColoUp-1 in a biological sample obtained from the patient, comparing the ColoUp-1 expression level in the patient to a median ColoUp-1 expression level in a population of colon cancer patients, and modifying the treatment regimen when the of ColoUp-1 expression level in the biological sample obtained from the patient is above the median ColoUp-1 expression level. In other embodiments, the method involves providing a treatment regimen to the patient; measuring the ColoUp-1 expression level in a biological sample obtained from the patient receiving a treatment regimen; comparing said ColoUp-1 expression level to a median ColoUp-1 expression level in a population of colon cancer patients; and modifying the treatment regimen when the ColoUp-1 expression level in the biological sample obtained from the patient is below the median ColoUp-1 expression level. In some embodiments, the treatment regimen is modified when the ColoUp-1 expression level in the biological sample obtained from the patient is above the 20^{th}, 30^{th}, 40^{th}, 50^{th}, 60^{th}, 70^{th}, 80^{th} or 90^{th} percentile of ColoUp-1 expression levels. In other embodiments, the treatment regimen is modified when the ColoUp-1 expression level in the biological sample obtained from the patient is below the 20^{th}, 30^{th}, 40^{th}, 50^{th}, 60^{th}, 70^{th}, 80^{th} or 90^{th} percentile of ColoUp-1 expression levels.

In some embodiments, the biological sample is obtained from the patient prior to the start of the treatment regimen. In some embodiments, the biological sample is obtained from the patient one week, two weeks, three weeks, one month, two months, three months, four months, five months, six months, nine months or one year after the start of the treatment regimen.

In some embodiments, the treatment regimen prior to the utilization of the prognostic methods disclosed herein is resection, anastomosis, radiation therapy or chemotherapy. In some embodiments, the treatment regimen for treating the colon cancer comprises the administration to the patient of fluorouracil, bevacizumab, irinotecan hydrochloride, capecitabine, cetuximab, oxaliplatin, leucovorin calcium, panitumumab, regorafenib, ziv-aflibercept, or any combination thereof. In some embodiments, the treatment regimen for treating the colon cancer comprises the administration to the patient of a compound that antagonizes ColoUp-1 function. In some embodiments, the compound that antagonizes ColoUp-1 function is an anti-ColoUp-1 antibody or a fragment thereof. In some embodiments, the anti-ColoUp-1 antibody or fragment thereof is humanized. The skilled worker is aware of additional compounds or therapies that may be used in a treatment regimen of colon cancer in a patient.

In some embodiments, modification of the treatment regimen comprises increasing the amount or level of treatments already administered to the patient. In particular embodiments, modification of the treatment regimen comprises increasing the dosage of a therapeutic compound (e.g., a small molecule, peptide, or antibody) or increasing the amount of radiation therapy already administered to the patient. In other embodiments, modification of the treatment regimen comprises increasing the frequency of administration of a therapeutic compound or radiation therapy to the patient. In some embodiments, the modification of the treatment regimen comprises decreasing the dosage of chemotherapy or decreasing the frequency of dosages of chemotherapy. In some embodiments, the modification of the treatment regimen comprises decreasing the amount of radiation therapy or decreasing the frequency of radiation therapy sessions. In some embodiments, the modification of the treatment regimen comprises discontinuing chemotherapy or radiation therapy. In other embodiments, modification of the treatment regimen comprises switching from one type of treatment regimen (e.g., chemotherapy or radiation therapy) to another type of treatment regimen (e.g., chemotherapy or radiation therapy). In other embodiments, modification of the treatment regimen comprises utilizing a form of treatment for the patient in addition to the treatment(s) already being provided to the patient before determination of the patient's ColoUp-1 expression levels. In some embodiments, modification of the treatment regimen comprises the utilization of resection, anastomosis, radiation therapy or chemotherapy. In some embodiments, modification of the treatment regimen comprises the administration to the patient of fluorouracil, bevacizumab, irinotecan hydrochloride, capecitabine, cetuximab, oxaliplatin, leucovorin calcium, panitumumab, regorafenib, ziv-aflibercept, or any combination thereof. In some embodiments, modification of the treatment regimen comprises the administration to the patient of a compound that antagonizes ColoUp-1 function. In some embodiments, the compound that antagonizes ColoUp-1 function is an anti-ColoUp-1 antibody or a fragment thereof. In some embodiments, the anti-ColoUp-1 antibody or fragment thereof is humanized. The skilled worker is aware of additional compounds or therapies that may be used in a modified treatment regimen of colon cancer in a patient.

In certain embodiments, the biological sample is obtained from the patient prior to the start of the treatment regimen. In certain embodiments, the biological sample is obtained from the patient 0, 1, 2, 3, 4, 5, 6, 7, 8, 9 or 10 months after the start of the treatment regimen. In certain embodiments, the biological sample is obtained from the patient 1-10 months after the start of the treatment regimen. In certain embodiments, the biological sample is obtained from the patient 1-2, 1-3, 2-4, 3-5, 4-6, 5-7, 6-8, or 9-10 months after the start of the treatment regimen.

Samples for use with the methods described herein may be essentially any biological material of interest. For example, a sample may be a tissue sample from a subject, a fluid sample from a subject, a solid or semi-solid sample from a subject, a primary cell culture or tissue culture of materials derived from a subject, cells from a cell line, or medium or other extracellular material from a cell or tissue culture, or a xenograft (meaning a sample of a colon cancer from a first subject, e.g. a human, that has been cultured in a second subject, e.g. an immunocompromised mouse). The term “sample” as used herein is intended to encompass both a biological material obtained directly from a subject (which may be described as the primary sample) as well as any manipulated forms or portions of a primary sample. For example, in certain embodiments, a preferred fluid sample is a blood sample. In this case, the term sample is intended to encompass not only the blood as obtained directly from the patient but also fractions of the blood, such as plasma, serum, protein preparations, nucleic acid preparations, etc. Furthermore, the term “sample” is intended to encompass the primary sample after it has been mixed with one or more additive, such as preservatives, chelators, anti-clotting factors, etc.

In certain embodiments, a fluid sample is a urine sample. In certain embodiments, a preferred solid or semi-solid sample is a stool sample. In certain embodiments, a preferred tissue sample is a sample of a tumor. Tumor samples may be obtained from tumor resection, from a biopsy from a tissue known to harbor or suspected of harboring a colon cancer tumor, from collection of circulating tumor cells, or by other methods. In certain embodiments, a preferred cell culture sample is a sample comprising cultured cells of a colon cancer cell line, such as a cell line cultured from a metastatic or a non-metastatic colon cancer tumor or a colon-derived cell line lacking a functional TGF-β, TGF-β receptor or TGF-β signaling pathway. A subject is preferably a human subject, but it is expected that the molecular markers disclosed herein, and particularly their homologs from other animals, are of similar utility in other animals. In certain embodiments, it may be possible to detect a marker directly in an organism without obtaining a separate portion of biological material. In such instances, the term sample is intended to encompass that portion of biological material that is contacted with a reagent or device involved in the detection process.

As mentioned above, the methods of the disclosure include comparing the level of ColoUp-1 expression in a colon cancer patient to the median ColoUp-1 expression level in a population of colon cancer patients. The population of colon cancer patients may be a population of patients afflicted with any Stage of colon cancer. In certain embodiments, the population of colon cancer patients is a population of patients afflicted with Stage II to IV colon cancer. In certain embodiments, the population of colon cancer patients is a population of patients afflicted with Stage II cancer. In other embodiments, the population of colon cancer patients is a population of patients afflicted with Stage III cancer. In other embodiments, the population of colon cancer patients is a population of patients afflicted with Stage IV cancer.

In some aspects, it may be advantageous to compare the ColoUp-1 expression level of the colon cancer patient to the median ColoUp-1 expression level of a population of patients afflicted with the same Stage of colon cancer as the patient. By way of example, the method may include obtaining a sample from a patient afflicted with Stage III colon cancer, measuring the ColoUp-1 expression level in the biological sample of the patient, determining a median expression level from a population of patients afflicted with Stage III colon cancer, and subsequently comparing the ColoUp-1 expression level from the colon cancer patient's biological sample to the median ColoUp-1 expression level obtained from the population of colon cancer patients.

Accordingly, the methods of the disclosure may comprise comparing the ColoUp-1 expression level in a patient afflicted with Stage II, Stage III or Stage IV colon cancer to the median ColoUp-1 expression level in a population of patients afflicted with Stage II, Stage III or Stage IV colon cancer, respectively.

Additionally, the patient and the population of patients may also share other similarities, such as similarities in previous treatment regimens, lifestyle (e.g., smokers or nonsmokers, overweight or underweight), or other demographics (e.g., age, genetic disposition). For example, besides having the same type of tumor, the patient and the population of patients may not have received any previous systemic chemotherapy. Furthermore, a population of colon cancer patients should comprise at least 10 subjects, e.g., from 15, 20, 25, 30, 40, 50, 60, 70, 80, 90, 100, 120, 140, 160, 180, or 200 or more subjects.

In some embodiments, the median ColoUp-1 expression level of the colon cancer population is 6-18 fold above the level of ColoUp-1 expression in a reference sample. In other embodiments, the median ColoUp-1 expression level is 6, 7, 8, 9, 10, 11, 12, 13, 14, 15, 16, 17 or 18 above the level of ColoUp-1 expression in a reference sample. In some embodiments, the median ColoUp-1 expression level is 6.5-7.5, 7-8, 7.5-8.5, 8-9, 8.5-9.5, 9-10, 9.5-10.5, 10-11, 10.5-11.5, 11-12, 11.5-12.5, 12-13, 13.5-14.5, 13-14, 14.5-15.5, 15-16, 15.5-16.5, 16-17, 16.5-17.5, or 17-18 fold above the level of ColoUp-1 expression in a reference sample. Preferably, the reference sample is a non-tumor sample. More preferably, the reference sample is a non-tumor colon mucosa sample.

In other embodiments, the median ColoUp-1 expression level of the colon cancer population is 6-18 fold above the detection limit (or detection threshold) of the assay used to measure ColoUp-1 expression. In certain embodiments, the median ColoUp-1 expression level is 13.2 fold above the detection limit of the assay. In other embodiments, the median ColoUp-1 expression level is 6, 7, 8, 9, 10, 11, 12, 13, 14, 15, 16, 17 or 18 above the detection limit of the assay. In some embodiments, the median ColoUp-1 expression level is 6.5-7.5, 7-8, 7.5-8.5, 8-9, 8.5-9.5, 9-10, 9.5-10.5, 10-11, 10.5-11.5, 11-12, 11.5-12.5, 12-13, 13.5-14.5, 13-14, 14.5-15.5, 15-16, 15.5-16.5, 16-17, 16.5-17.5, or 17-18 fold above the detection limit of the assay.

Alternatively, the methods of the disclosure may comprise comparing the ColoUp-1 expression level in a patient afflicted with Stage II, Stage III or Stage IV colon cancer to the median ColoUp-1 expression level in a reference population of patients afflicted with Stage III colon cancer, or to a ColoUp-1 expression value between the 40^{th}-60^{th} percentile of expression in a reference population of patients afflicted with Stage III colon cancer, or to a Colo-Up1 expression value between the 30^{th}-70^{th} percentile of expression in a reference population of patients afflicted with Stage III colon cancer. In some embodiments, the method of prognosis comprises the steps of measuring the ColoUp-1 expression level in a biological sample obtained from a colon cancer patient; and comparing said ColoUp-1 expression level in the biological sample to a ColoUp-1 expression level in a population of colon cancer patients; wherein a ColoUp-1 expression level in the biological sample greater than the ColoUp-1 expression level of the 25^{th}, 30^{th}, 40^{th}, 50^{th}, 60^{th}, 70^{th}, 80^{th}, 90^{th} or 95^{th} percentile of the population of colon cancer patients is indicative of poor survival prognosis. In some embodiments, the method of prognosis comprises the steps of measuring the ColoUp-1 expression level in a biological sample obtained from a colon cancer patient; and comparing said ColoUp-1 expression level in the biological sample to a ColoUp-1 expression level in a population of colon cancer patients; wherein a ColoUp-1 expression level in the biological sample greater than a ColoUp-1 expression level of the 25^{th}-75^{th}, 30^{th}-70^{th}, or 40^{th} to 60^{th} percentile of the population of colon cancer patients is indicative of poor survival prognosis.

In some embodiments, the method is a method of determining a Stage II or a Stage III colon cancer prognosis comprising: measuring the ColoUp-1 expression level in a biological sample obtained from a colon cancer patient; and comparing said ColoUp-1 expression level in the biological sample to a ColoUp-1 expression level in a population of colon cancer patients; wherein a ColoUp-1 expression level in the biological sample greater than the ColoUp-1 expression level of the 25^{th}, 40^{th}, 50^{th}, 60^{th} or 70^{th} percentile of the population of colon cancer patients is indicative of poor survival prognosis. In some embodiments, a ColoUp-1 expression level in the biological sample greater than the ColoUp-1 expression level of the 25^{th}-75^{th}, 30^{th}-70^{th}, or 40^{th}-60^{th} percentile of the population of colon cancer patients is indicative of poor survival prognosis. In some embodiments, the population of colon cancer patients is a population of colon cancer patients with Stage III colon cancer.

In further embodiments, the median ColoUp-1 expression level of the population may be about 330 as measured by microarray analysis. In some embodiments, the median expression is 200-800, 220-800, 230-800, 240-800, 250-800, 200-600, 300-500, 325-400 or 325-375. In some embodiments, the median expression level is about 200, 250, 300, 330, 350, 400, 450, 500, 550, 600, 650, 700, 750, or 800.

In some embodiments, the median ColoUp-1 expression level of the population may be between 1.024 and 1.047 as measured by real-time PCR. In some embodiments the median value of expression is 0.6-1.5, 0.8-1.2, or 0.9-1.1. In some embodiments, the median expression is 1.000-1.070, 1.010-1.060 or 1.020-1.050. In some embodiments, the median ColoUp1 expression level of the population is 1.02-1.025, 1.02-1.03, 1.04-1.05, 1.045-1.05. In some embodiments, the median ColoUp1 expression level of the population is 1.024 or 1.047.

Poor survival prognosis according to certain embodiments of the present disclosure includes a survival time of less than 60, 50, 40, 35 or 30 months after obtaining the biological sample from the patient. In further embodiments, good survival prognosis includes a survival time of more than 60, 70, 80, 90, 100, or 140 months after obtaining the biological sample from the patient.

It will be recognized by one of skill in the art that levels of ColoUp expression can be also be determined by measuring portions or fragments of the full length transcript or protein.

### 4. Methods of Measuring ColoUp-1 Expression Levels

In certain embodiments, a method of the disclosure comprises measuring the level of ColoUp-1 nucleic acid, such as an mRNA, in a sample. Optionally, the method involves obtaining a quantitative measure of the ColoUp-1 expressed nucleic acid in the sample. Accordingly, in some aspects, the application provides isolated or recombinant nucleotide sequences that are at least 80%, 85%, 90%, 95%, 97%, 98%, 99% or 100% identical to the ColoUp-1 nucleic acid sequence (SEQ ID NO: 4). One of ordinary skill in the art will appreciate that ColoUp-1 nucleic acid sequences complementary to SEQ ID NO: 4 and variants thereof are also within the scope of this invention. Such variant nucleotide sequences include sequences that differ by one or more nucleotide substitutions, additions or deletions, such as allelic variants.

In view of this specification, one of skill in the art will recognize a wide range of techniques that may be employed to detect and optionally quantitate the presence of a nucleic acid. Nucleic acid detection systems generally involve preparing a purified nucleic acid fraction of a sample, and subjecting the sample to a direct detection assay or an amplification process followed by a detection assay. Amplification may be achieved, for example, by polymerase chain reaction (PCR), reverse transcriptase (RT) and coupled RT-PCR. Detection of a nucleic acid is generally accomplished by probing the purified nucleic acid fraction with a probe that hybridizes to the nucleic acid of interest, and in many instances detection involves an amplification as well. Northern blots, dot blots, microarrays, quantitative PCR and quantitative RT-PCR or real-time PCR, are all well known methods for detecting a nucleic acid in a sample.

In certain embodiments, mRNA expression data is gathered by a highly parallel system, meaning a system that allows simultaneous or near-simultaneous collection of expression data for one hundred or more gene transcripts. Exemplary highly parallel systems include probe arrays (“arrays”) that are often divided into microarrays and microarrays, where microarrays have a much higher density of individual probe species per area. Arrays generally consist of a surface to which probes that correspond in sequence to gene products (e.g., cDNAs, mRNAs, oligonucleotides) are bound at known positions. The probes can be, e.g., a synthetic oligomer, a full-length cDNA, a less-than full length cDNA, or a gene fragment. Usually a microarray will have probes corresponding to at least 100 gene products and more preferably, 500, 1000, 4000 or more. Probes may be small oligomers or larger polymers, and there may be a plurality of overlapping or non-overlapping probes for each transcript.

The nucleic acids to be contacted with the microarray may be prepared in a variety of ways. Methods for preparing total and poly(A)+ RNA are well known and are described generally in Sambrook et al., supra. Labeled cDNA may be prepared from mRNA by oligo dT-primed or random-primed reverse transcription, both of which are well known in the art (see e.g., Klug and Berger, 1987, Methods Enzymol. 152:316-325). cDNAs may be labeled by incorporation of labeled nucleotides or by labeling after synthesis. Preferred labels are fluorescent labels.

Nucleic acid hybridization and wash conditions are chosen so that the population of labeled nucleic acids will specifically hybridize to appropriate, complementary probes affixed to the matrix. Optimal hybridization conditions will depend on the length (e.g., oligomer versus polynucleotide greater than 200 bases) and type (e.g., RNA, DNA, PNA) of labeled nucleic acids and immobilized polynucleotide or oligonucleotide. General parameters for specific (i.e., stringent) hybridization conditions for nucleic acids are described in Sambrook et al., supra, and in Ausubel et al., 1987, Current Protocols in Molecular Biology, Greene Publishing and Wiley-Interscience, New York, which is incorporated in its entirety for all purposes. Non-specific binding of the labeled nucleic acids to the array can be decreased by treating the array with a large quantity of non-specific DNA—a so-called “blocking” step.

Signals, such as fluorescent emissions for each location on an array are generally recorded, quantitated and analyzed using a variety of computer software. Signal for any one gene product may be normalized by a variety of different methods. Arrays preferably include control and reference probes. Control probes are nucleic acids which serve to indicate that the hybridization was effective. Reference probes allow the normalization of results from one experiment to another, and to compare multiple experiments on a quantitative level. Reference probes are typically chosen to correspond to genes that are expressed at a relatively constant level across different cell types and/or across different culture conditions. Exemplary reference nucleic acids include housekeeping genes of known expression levels, e.g., GAPDH, hexokinase and actin. Other reference nucleic acids include any one of DDA1, SAC3D1, TMEM160, CPNE2, TMEM134, ZNF787, ZNF746, SIRT3. In particular embodiments, the reference transcript is CPNE2, SAC3D1, and/or TMEM160.

In some embodiments, the ColoUp-1 expression level (e.g., as determined by RT-PCR) is normalized against a reference standard generated from the expression levels of a set of reference transcripts. In some embodiments, the set of reference transcripts comprises candidates with the most stable expression values across primary colon cancer samples. In some embodiments, the reference transcript is any one of DDA1, SAC3D1, TMEM160, CPNE2, TMEM134, ZNF787, ZNF746, SIRT3. In particular embodiments, the reference transcript is CPNE2, SAC3D1, and/or TMEM160. In some embodiments, levels of CPNE2 are measured by utilizing RT-PCR and amplifying a PCR product spanning the exon 14/15 boundary of the CPNE2 gene. In some embodiments, levels of CPNE2 are measured by utilizing RT-PCR and amplifying a product comprising the sequence of SEQ ID NO: 9. In some embodiments, levels of CPNE2 are measured by utilizing RT-PCR and amplifying a PCR product that is 50-100 (e.g., at least 50, 60, 64, 65, 70, 75, 80, 85, 90, 95, 100 base pairs) in length and having a sequence midpoint corresponding to base pair 1588 of SEQ ID NO: 12. In some embodiments, CPNE2 levels are measured by utilizing a primer or probe that binds to SEQ ID NO: 9. In some embodiments, levels of SAC3D1 are measured by utilizing RT-PCR and amplifying a PCR product spanning the exon 1/2 boundary of the SAC3D1 gene. In some embodiments, levels of SAC3D1 are measured by utilizing RT-PCR and amplifying a product comprising the sequence of SEQ ID NO: 10. In some embodiments, levels of SAC3D1 are measured by utilizing RT-PCR and amplifying a PCR product that is 50-100 (e.g., at least 50, 60, 65, 69, 70, 75, 80, 85, 90, 95, 100 base pairs) in length and having a sequence midpoint corresponding to base pair 966 of SEQ ID NO: 13. In some embodiments, SAC3D1 levels are measured by utilizing a primer or probe that binds to SEQ ID NO: 10. In some embodiments, levels of TMEM160 are measured by utilizing RT-PCR and amplifying a PCR product spanning the exon 1/2 boundary of the TMEM160 gene. In some embodiments, levels of TMEM160 are measured by utilizing RT-PCR and amplifying a product comprising the sequence of SEQ ID NO: 11. In some embodiments, levels of TMEM160 are measured by utilizing RT-PCR and amplifying a PCR product that is 50-100 (e.g., at least 50, 58, 60, 65, 70, 75, 80, 85, 90, 95, 100 base pairs) in length and having a sequence midpoint corresponding to base pair 217 of SEQ ID NO: 14. In some embodiments, TMEM160 levels are measured by utilizing a primer or probe that binds to SEQ ID NO: 11. In some embodiments, the hydrolysis probe/primer sets for CPNE2 (NM_{—}152727), SAC3D1 (NM_{—}013299), and TMEM160 (NM_{—}017854) are Hs00541611_m1, Hs01017027_m1, and Hs00215289_m1 respectively, from Applied Biosystems. In some embodiments, the primers used for measuring the levels of the reference transcript are 15-35, 15-25, 18-24 or 18-22 nucleotides in length. In some embodiments, the primers used for measuring the reference transcript are 18 nucleotides in length. In some embodiments, gene expression profiles of more than 40, 50, 60, or 70 primary colon cancer samples are analyzed. In some embodiments, the expression levels are measured using real-time PCR. In some embodiments, the mean of the expression values of one or more reference transcripts is used as the reference. In some embodiments, the mean is the arithmetic mean. In some embodiments, the mean refers to the geometric mean.

In some embodiments, (Cq_{GEOX}), the geometric mean of the Cq values for at least 2, 3, 4, 5, 6, 7, 8, 9 or 10 different reference sequences, (e.g., CPNE2, SAC3D1 and TMEM160), are used for normalization. In some embodiments, the level of ColoUp-1 expression is determined as the ratio of ColoUp-1-1:GEOX=2exp−(Cq_{ColoUp-1}−Cq_{GEOX}). In some embodiments, for each reverse transcription reaction (e.g., Cq_{ColoUp-1}, Cq_{reference sequence}), values may be determined as the average values obtained from at least three, four, five, six, seven, eight, nine or ten independent real-time PCR reactions. In some embodiments, the (Cq_{GEOX}), the geometric mean of the Cq values for 3, 4, 5, 6, 7, 8, 9 or 10 different reference sequences, (e.g., CPNE2, SAC3D1 and TMEM160), are used for normalization. In some embodiments, (Cq_{GEOX}), the geometric mean of the Cq values for three different reference sequences, (i.e., CPNE2, SAC3D1 and TMEM160), are used for normalization.

In some embodiments, ColoUp-1 is measured by amplifying a nucleic acid sequence comprising the nucleotide sequences of any one of SEQ ID NOs: 5-8. In some embodiments, ColoUp-1 is measured by amplifying a product that has the sequence of SEQ ID NO: 6 or 8. In some embodiments, ColoUp-1 transcript is measured by using primers, wherein at least one of the primers binds to a sequence of any one of SEQ ID NOs: 5-8. In some embodiments, ColoUp-1 is measured by real-time PCR using hydrolysis probe/primer set Hs00378520_m1 from Applied Biosystems. In some embodiments, ColoUp-1 is measured by real-time PCR using hydrolysis probe/primer set Hs00378530_m1 from Applied Biosystems. In some embodiments, the primers used for measuring the levels of the ColoUp-1 transcript are 15-35, 15-25, 18-24 or 18-22 nucleotides in length. In some embodiments, the primers used for measuring the ColoUp-1 are 18 nucleotides in length.

A number of methods for constructing or using arrays are described in the following references. Schena et al., 1995, Science 270:467-470; DeRisi et al., 1996, Nature Genetics 14:457-460; Shalon et al., 1996, Genome Res. 6:639-645; Schena et al., 1995, Proc. Natl. Acad. Sci. USA 93:10539-11286; Fodor et al., 1991, Science 251:767-773; Pease et al., 1994, Proc. Natl. Acad. Sci. USA 91:5022-5026; Lockhart et al., 1996, Nature Biotech 14:1675; U.S. Pat. Nos. 6,051,380; 6,083,697; 5,578,832; 5,599,695; 5,593,839; 5,631,734; 5,556,752; 5,510,270; EP No. 0 799 897; PCT No. WO 97/29212; PCT No. WO 97/27317; EP No. 0 785 280; PCT No. WO 97/02357; EP No. 0 728 520; EP No. 0 721 016; PCT No. WO 95/22058.

A variety of companies provide microarrays and software for extracting certain information from microarray data. Such companies include Affymetrix (Santa Clara, Calif.), GeneLogic (Gaithersburg, Md.) and Eos Biotechnology Inc. (South San Francisco, Calif.).

While the above discussion focuses on the use of arrays for the collection of mRNA expression data, such data may also be obtained through a variety of other methods, that, in view of this specification, are known to one of skill in the art. For example, real-time PCR is an amplification technique that can be used to determine levels of mRNA expression. Real-time PCR evaluates the level of PCR product accumulation during amplification. This technique permits quantitative evaluation of mRNA levels in multiple samples. For mRNA levels, mRNA is extracted from a biological sample, e.g. a tumor and normal tissue, and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes can be obtained commercially from, for example, Perkin Elmer/Applied Biosystems (Foster City, Calif.). To quantitate the amount of the specific nucleic acid of interest in a sample, a standard curve is generated using a control. Standard curves can be generated using the Ct or Cq values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

In some embodiments, TaqMan based assays are used to measure mRNA levels. Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

The TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification (see, for example, perkin-elmer.com).

In other embodiments, real-time quantitative PCR can be performed using intercalating fluorescent dyes like SYBR Green I and measuring the signal intensity after amplification, which can be assayed for example in the LightCycler Real Time PCR System (Roche) or ABI 7900HT Fast Real Time PCR System (Applied Biosystems).

In other embodiments, detection of RNA transcripts can be achieved by Northern blotting, wherein a preparation of RNA is run on a denaturing agarose gel, and transferred to a suitable support, such as activated cellulose, nitrocellulose or glass or nylon membranes. Labeled (e.g., radiolabeled) cDNA or RNA is then hybridized to the preparation, washed and analyzed by methods such as autoradiography.

Detection of RNA transcripts can further be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap lipase chain reaction (RT-AGLCR) as described by R. L. Marshall, et al., PCR Methods and Applications 4: 80-84 (1994). One suitable method for detecting enzyme mRNA transcripts is described in reference Pabic et. al. Hepatology, 37(5): 1056-1066, 2003.

The application also provides methods of measuring the amount of ColoUp-1 protein in a biological sample. Optionally, the method involves obtaining a quantitative measure of ColoUp-1 protein in the sample. In view of this specification, one of skill in the art will recognize a wide range of techniques that may be employed to detect and optionally quantitate the presence of a protein. In preferred embodiments, ColoUp-1 protein is detected with an antibody. The antibody may be specific for different forms of ColoUp-1. For example, the antibody may specifically detect a 150 kDa ColoUp-1 protein, while another antibody may specifically detect a 100 kDa ColoUp-1 protein. Suitable antibodies are described in a separate section below.

In many embodiments, an antibody-based detection assay involves bringing the sample and the antibody into contact so that the antibody has an opportunity to bind to proteins having the corresponding epitope. In many embodiments, an antibody-based detection assay also typically involves a system for detecting the presence of antibody-epitope complexes, thereby achieving a detection of the presence of the proteins having the corresponding epitope. Antibodies may be used in a variety of detection techniques, including enzyme-linked immunosorbent assays (ELISAs), immunoprecipitations, Western blots.

Antibody-independent techniques for identifying a protein may also be employed. For example, mass spectroscopy, particularly coupled with liquid chromatography, permits detection and quantification of large numbers of proteins in a sample. Two-dimensional gel electrophoresis may also be used to identify proteins, and may be coupled with mass spectroscopy or other detection techniques, such as N-terminal protein sequencing. RNA aptamers with specific binding for the protein of interest may also be generated and used as a detection reagent.

The application also provides recombinant, isolated, substantially purified or purified ColoUp-1 protein, or fragment thereof. The ColoUp-1 polypeptides may also include one or more post-translational modifications, such as glycosylation, phosphorylation, lipid modification, acetylation, etc.

In certain embodiments, the application provides isolated, substantially purified, purified or recombinant polypeptides comprising an amino acid sequence that is at least 90% identical to an amino acid sequence of any of SEQ ID Nos: 1, 2 or 3 and optionally at least 95%, 97%, 98%, 99%, 99.3%, 99.5% or 99.7% identical to an amino acid sequence of any of SEQ ID Nos: 1, 2 or 3.

Optionally, a ColoUp-1 of the disclosure comprises an additional moiety, such as an additional polypeptide sequence or other added compound, with a particular function, such as an epitope tag that facilitates detection of the recombinant polypeptide with an antibody, a purification moiety that facilitates purification (e.g. by affinity purification), a detection moiety, that facilitates detection of the polypeptide in vivo or in vitro, or an antigenic moiety that increases the antigenicity of the polypeptide so as to facilitate antibody production. Often, a single moiety will provide multiple functionalities. For example, an epitope tag will generally also assist in purification, because an antibody that recognizes the epitope can be used in an affinity purification procedure as well. Examples of commonly used epitope tags are: an HA tag, a hexahistidine tag, a V5 tag, a Glu-Glu tag, a c-myc tag, a VSV-G tag, a FLAG tag, an enterokinase cleavage site tag and a T7 tag. Commonly used purification moieties include: a hexahistidine tag, a glutathione-S-transferase domain, a cellulose binding domain and a biotin tag. Commonly used detection moieties include fluorescent proteins (e.g. green fluorescent proteins), a biotin tag, and chromogenic/fluorogenic enzymes (e.g. beta-galactosidase and luciferase). Commonly used antigenic moieties include the keyhole limpet hemocyanin and serum albumins. Note that these moieties need not be polypeptides and need not be connected to the polypeptide by a traditional peptide bond.

### 5. Antibodies and Uses Therefor

Another aspect of the disclosure pertains to an antibody specifically reactive with ColoUp-1 protein. For example, by using immunogens derived from a ColoUp-1 protein, e.g., based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a ColoUp-1 polypeptide or an antigenic fragment which is capable of eliciting an antibody response, or a fusion protein). Techniques for conferring immunogenicity on a protein or peptide include conjugation to carriers or other techniques well known in the art. An immunogenic portion of ColoUp-1 polypeptide can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of ColoUp-1 polypeptide of a mammal, e.g., antigenic determinants of a protein set forth in SEQ ID Nos: 1, 2 and 3.

In some embodiments, antibodies are specific for the protein as encoded by nucleic acid sequence as set forth in SEQ ID No: 4. In other embodiment, the antibodies are immunoreactive with one or more proteins having an amino acid sequence that is at least 80% identical to an amino acid sequence as set forth in SEQ ID Nos: 1, 2 or 3. In other embodiments, an antibody is immunoreactive with one or more proteins having an amino acid sequence that is at least 85%, 90%, 95%, 98%, 99%, 99.3%, 99.5%, 99.7% identical or 100% identical to an amino acid sequence as set forth in SEQ ID Nos: 1, 2 or 3. In certain preferred embodiments, the disclosure provides an antibody that binds to an epitope including the C-terminal portion of the polypeptide of SEQ ID Nos: 1, 2 or 3.

Following immunization of an animal with an antigenic preparation of a ColoUp-1 polypeptide, anti-ColoUp-1 antisera can be obtained and, if desired, polyclonal anti-ColoUp-1 antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian ColoUp-1 polypeptide of the present disclosure and monoclonal antibodies isolated from a culture comprising such hybridoma cells. In one embodiment anti-human ColoUp-1 antibodies specifically react with the protein encoded by a nucleic acid having SEQ ID No: 4.

Anti-ColoUp-1 antibodies can be used, e.g., to detect ColoUp-1 polypeptides in biological samples and/or to monitor ColoUp-1 polypeptide levels in an individual. The level of ColoUp polypeptide may be measured in a variety of sample types such as, for example, in cells, tumor samples, stools, and/or in bodily fluid, such as in whole blood samples, blood serum, blood plasma and urine. The adjective “specifically reactive with” as used in reference to an antibody is intended to mean, as is generally understood in the art, that the antibody is sufficiently selective between the antigen of interest (e.g. a ColoUp-1 polypeptide) and other antigens that are not of interest that the antibody is useful for, at minimum, detecting the presence of the antigen of interest in a particular type of biological sample.

In some embodiments, the anti-ColoUp-1 antibodies disclosed herein can be used for treating a patient having a colon neoplasia. In some embodiments, the anti-ColoUp-1 antibodies neutralize or antagonize the activity of ColoUp-1 in a colon neoplasia. In some embodiments, the colon neoplasia is colon cancer. In some embodiments, the anti-ColoUp-1 antibody is administered to patients prior to determining the patient's ColoUp-1 levels. In other embodiments, the anti-ColoUp-1 antibody is administered to patients after determining the patient's ColoUp-1 levels. In some embodiments, the anti-ColoUp-1 antibody is a humanized antibody.

In certain methods employing the antibody, a higher degree of specificity in binding may be desirable. For example, an antibody for use in detecting a low abundance protein of interest in the presence of one or more very high abundance protein that are not of interest may perform better if it has a higher degree of selectivity between the antigen of interest and other cross-reactants. Monoclonal antibodies generally have a greater tendency (as compared to polyclonal antibodies) to discriminate effectively between the desired antigens and cross-reacting polypeptides. In addition, an antibody that is effective at selectively identifying an antigen of interest in one type of biological sample (e.g. a stool sample) may not be as effective for selectively identifying the same antigen in a different type of biological sample (e.g. a blood sample). Likewise, an antibody that is effective at identifying an antigen of interest in a purified protein preparation that is devoid of other biological contaminants may not be as effective at identifying an antigen of interest in a crude biological sample, such as a blood or urine sample.

Accordingly, in preferred embodiments, the application provides antibodies that have demonstrated specificity for an antigen of interest (particularly, although not limited to, a ColoUp-1 polypeptide) in a sample type that is likely to be the sample type of choice for use of the antibody. In a particularly preferred embodiment, the application provides antibodies that bind specifically to a ColoUp-1 polypeptide in a protein preparation from blood (optionally serum or plasma) or tumor sample from a patient that has a colon cancer.

In addition, the techniques used to screen antibodies in order to identify a desirable antibody may influence the properties of the antibody obtained. For example, an antibody to be used for certain therapeutic purposes will preferably be able to target a particular cell type. Accordingly, to obtain antibodies of this type, it may be desirable to screen for antibodies that bind to cells that express the antigen of interest (e.g. by fluorescence activated cell sorting). Likewise, if an antibody is to be used for binding an antigen in solution, it may be desirable to test solution binding. A variety of different techniques are available for testing antibody:antigen interactions to identify particularly desirable antibodies. Such techniques include ELISAs, surface plasmon resonance binding assays (e.g. the Biacore binding assay, Bia-core AB, Uppsala, Sweden), sandwich assays (e.g. the paramagnetic bead system of IGEN International, Inc., Gaithersburg, Md.), western blots, immunoprecipitation assays and immunohistochemistry.

The invention now being generally described, it will be more readily understood by reference to the following examples, which are included merely for purposes of illustration of certain aspects and embodiments of the present invention, and are not intended to limit the invention.

### EXAMPLES

### Example 1

### Identification of ColoUp-1 and its Induction in Colon Cancer.

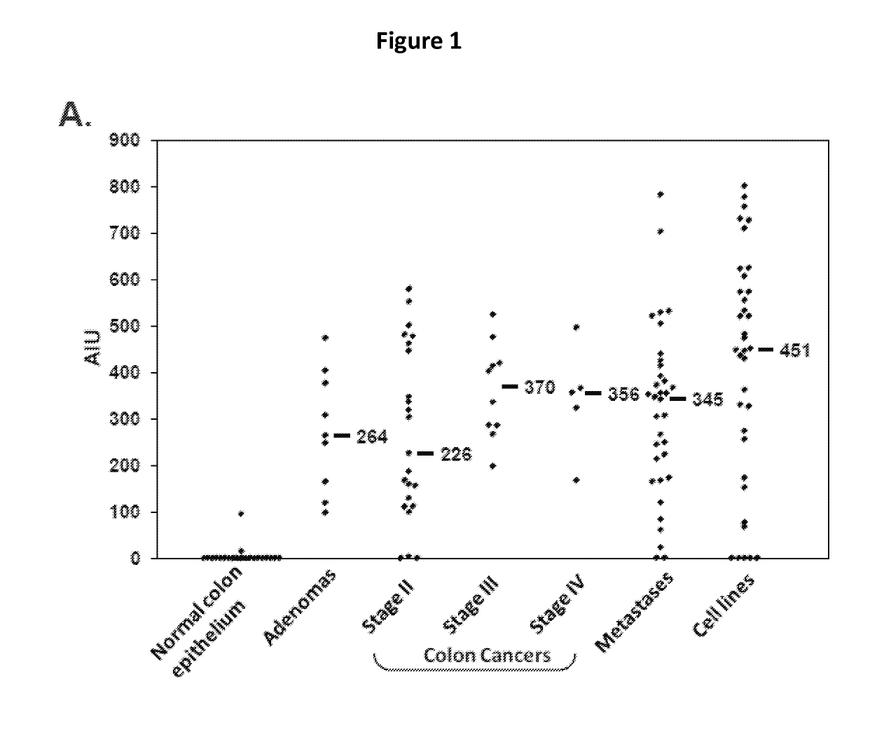

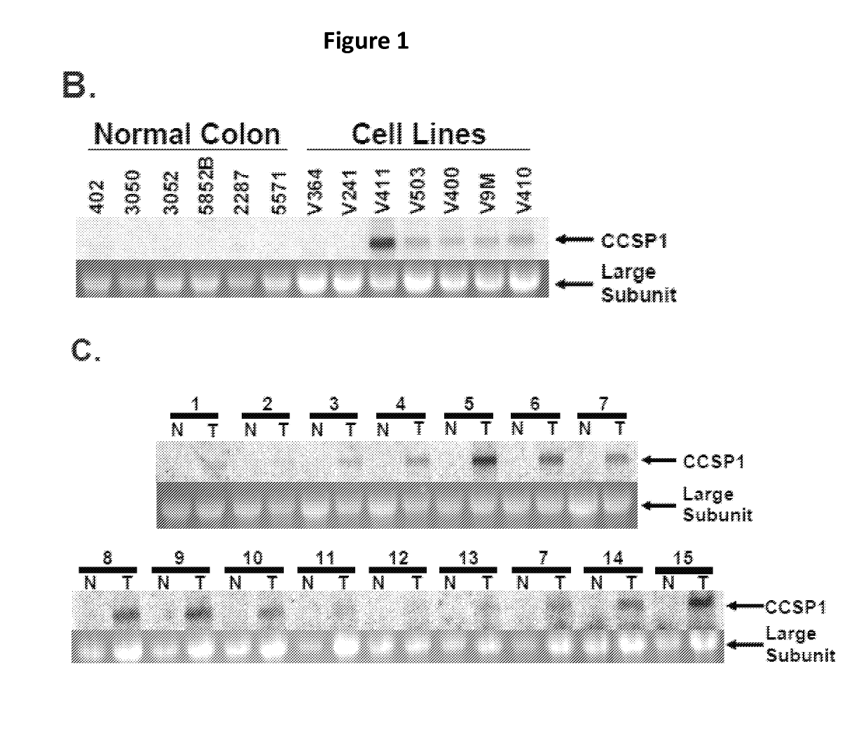

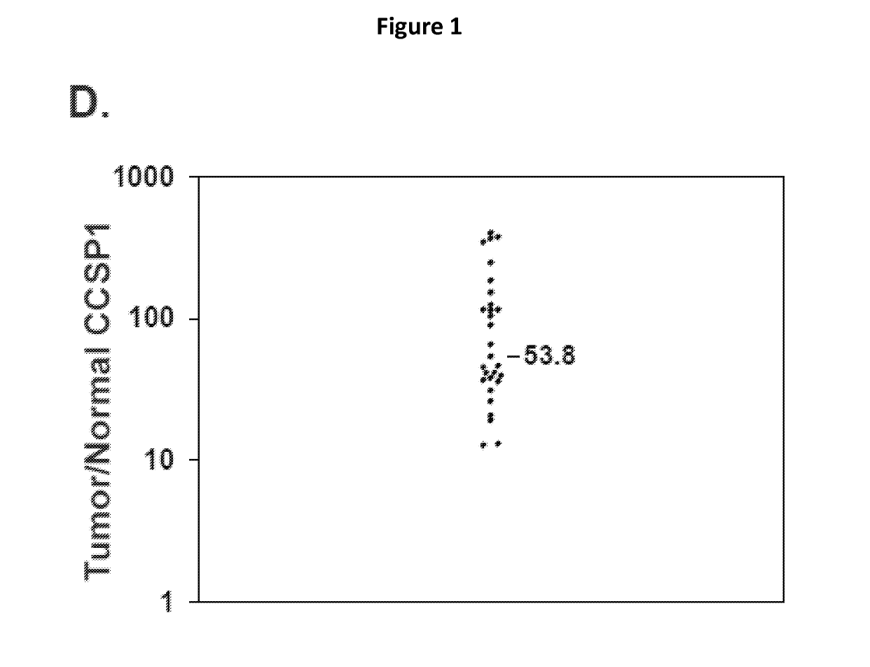

To identify novel candidate markers of colon cancer, GeneChip gene expression microarrays were used to compare patterns of gene expression in colon cancers versus normal colon epithelium. Twenty-one dissected colon epithelial strips from normal colonic mucosa were compared to a group of 72 colon cancer surgical specimens and 36 colon cancer cell lines on DNA microarrays that measure the expression of approximately 55,000 genes, EST clusters, and predicted exons. In this analysis, the two microarray probesets with the greatest induction in colon cancer cell lines versus normal colon mucosa were the probesets corresponding to P-Cadherin, a membrane protein known to be induced in colon cancer, and the probeset corresponding to a partial cDNA for a hypothetical gene called KIAA1199. As shown in FIG. 1, median expression of KIAA1199 was 451 in colon cancer cell lines, while only two of 21 normal colon mucosal samples showed any expression of KIAA1199 above the microarray measurement threshold of 25. Furthermore, KIAA1199 was well induced in 72 primary colon cancer tumors (median value 330) (FIG. 1A). Finally, induction of KIAA1199 is an early event in colon neoplasia with high levels of KIAA1199 detected in early node-negative Stage II colon cancers (median value 226), and in a set of colon adenomas (median value 264) (FIG. 1A).

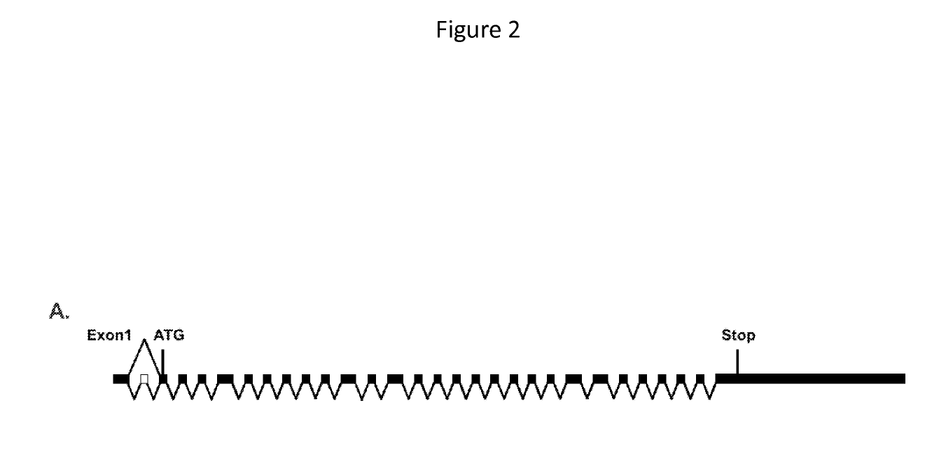

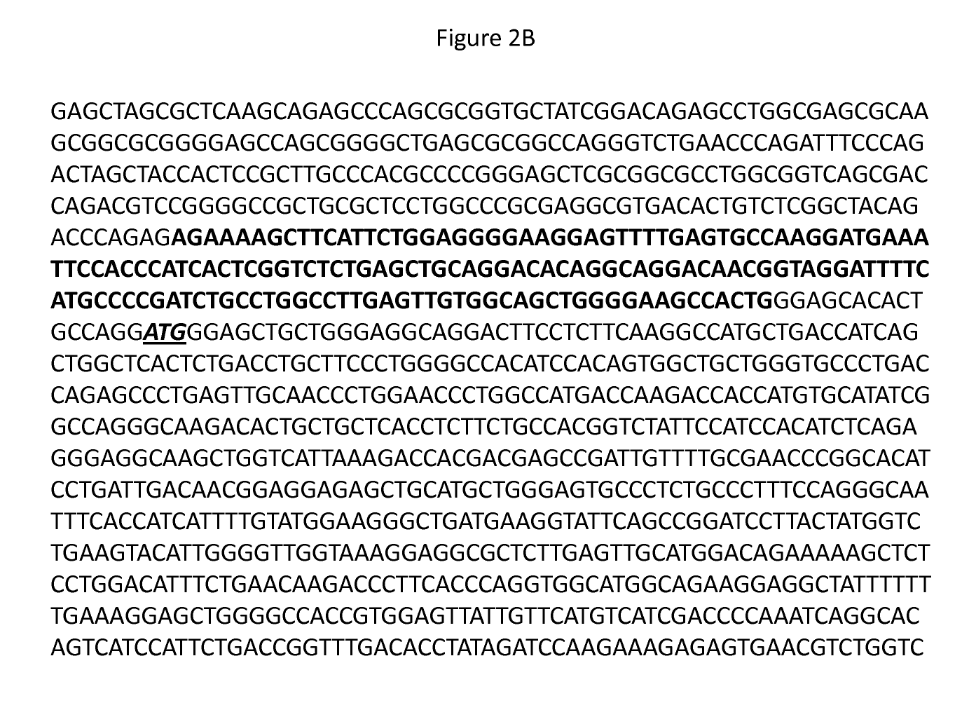

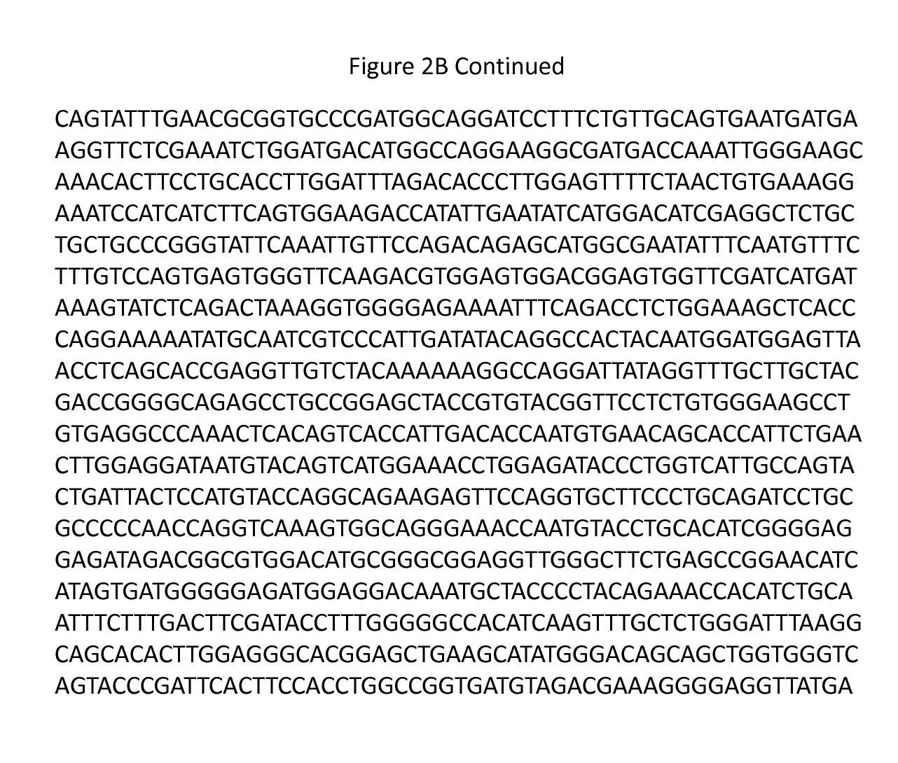

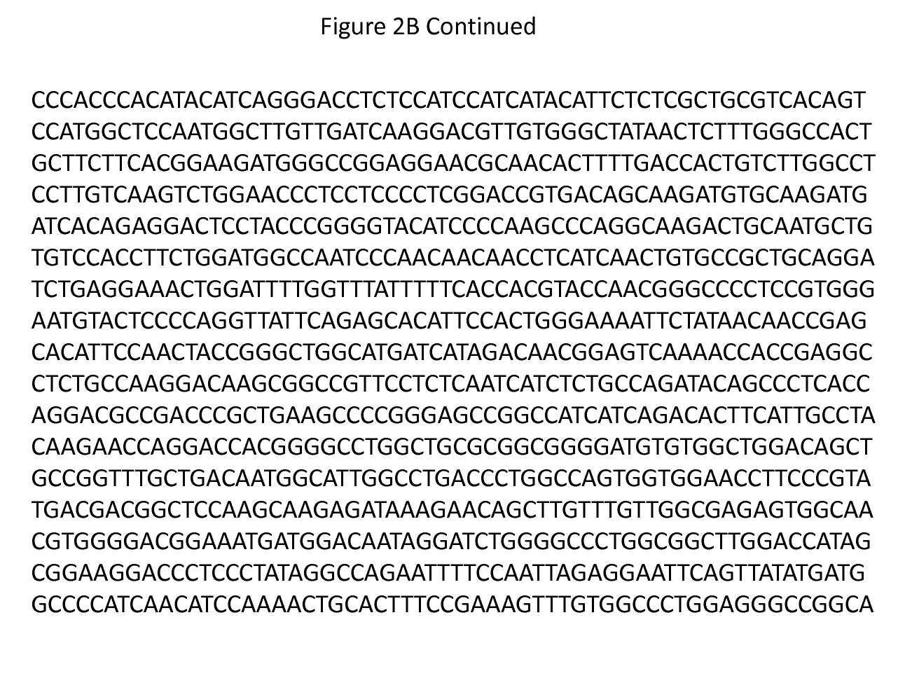

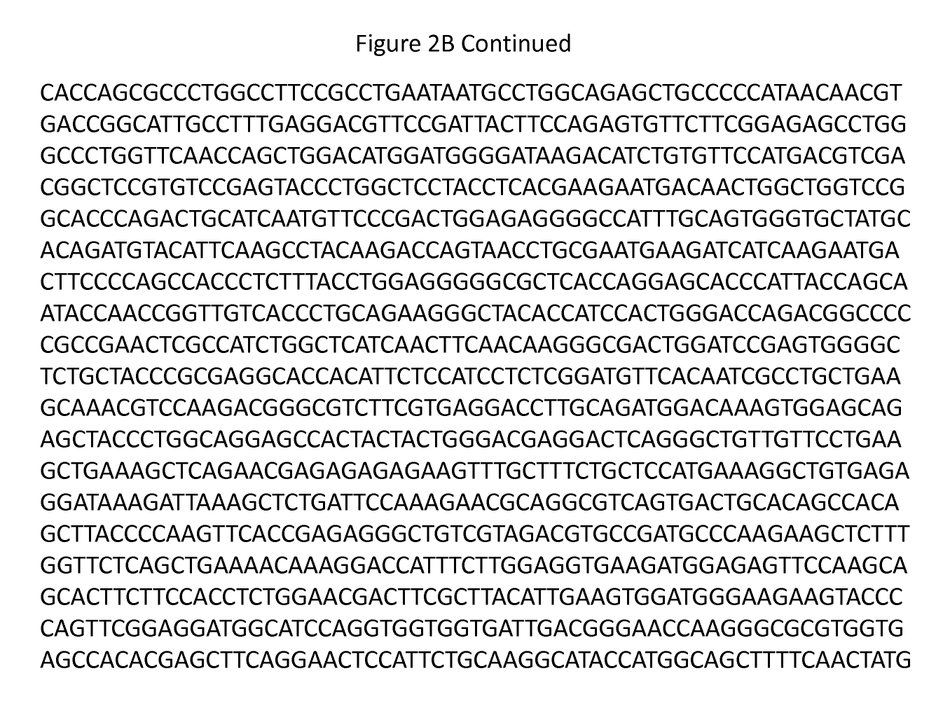

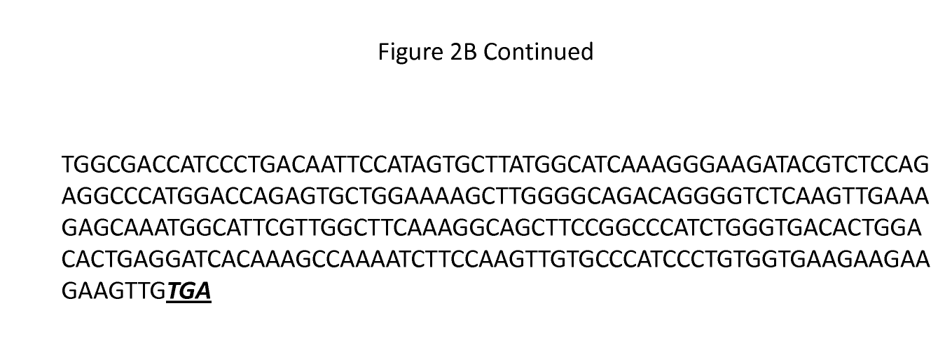

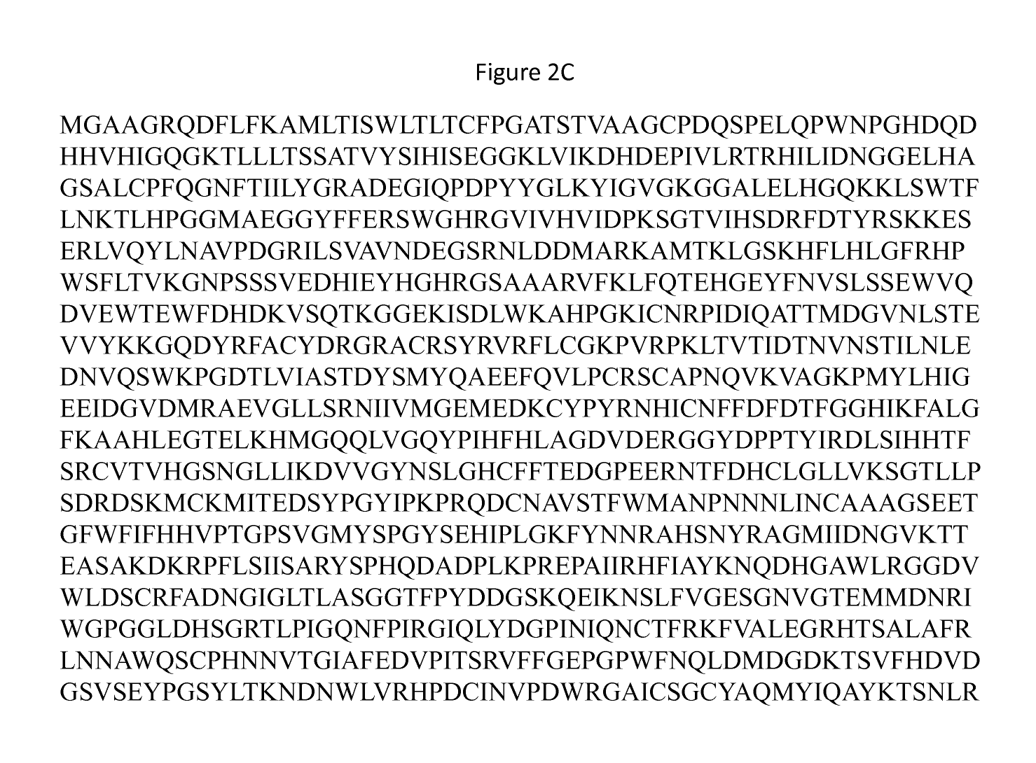

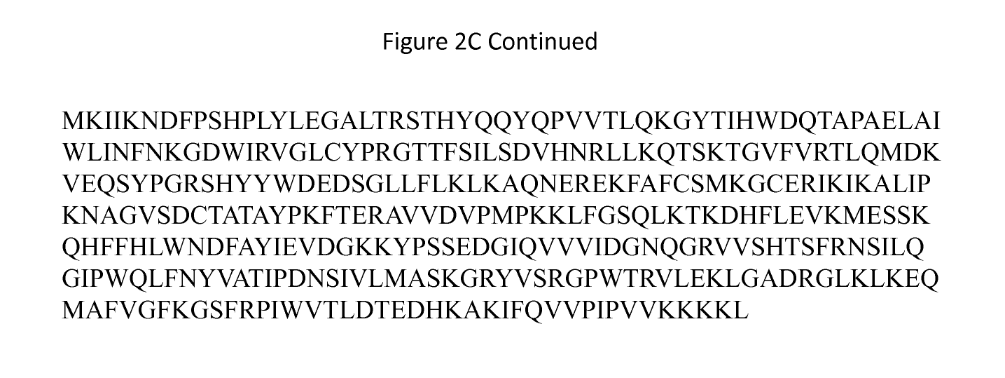

At the time of this initial study, only a partial cDNA of KIAA1199 of 5 kb was available, which contained a putative stop codon, but no start codon, and mapped to chromosome 15q. Using, RT-PCR partial KIAA1199 was connected to additional multiple ESTs that mapped to the 15q24-25 genomic region, resulting in a transcript containing a full-length coding region of 4083 bp that covers 30 exons and encodes a protein of 1361 amino acids (FIGS. 2A & 2C). It was also determined that there are at least two forms of the transcript that are approximately 7.0 and 7.2 kb in length, with the difference in length arising due to alternate splicing in the 5′ UTR (FIGS. 2A & 2B). The longer variant contains an extra exon of 159 bp. Both identified transcripts have in-frame TAG (7.0 kb form) or TGA (7.2 kb form) stop codon 5′ to the presumptive ATG start codon. The gene encoding these transcripts is herein called Colon Cancer Secreted Protein-1 (ColoUp-1). The colonic ColoUp-1 transcript agrees with the assembly of a cochlear transcript deposited as GenBank entry NM_{—}018689 by Abe et al.

### Example 2

### ColoUp-1 Expression is Commonly Induced in Colon Cancer Tissues and Cell Lines.

Northern blot analysis was employed to confirm the preliminary evidence from the GeneChip arrays of increased ColoUp-1 expression in colon cancer. As shown in FIG. 1B, Northern analysis strongly corroborated that ColoUp-1 is expressed by malignant but not normal colon tissues with a single 7 kb ColoUp-1 transcript of moderate to strong intensity being detected in 5 of 7 colon cancer cell lines, but in none of 6 normal colon epithelial tissue samples. Of note, the two cell lines testing negative for ColoUp-1 expression by Northern analysis had also tested as lacking in ColoUp-1 expression on the GeneChip microarray. Identical results were obtained on Northern analysis using hybridization probes spanning ColoUp-1 cDNA sequences from exons 1-9 (FIGS. 1B and 1C) or spanning exons 13-22 (data not shown).

ColoUp-1 expression was then measured in primary colon cancer tumors and compared to normal colon mucosa from the same patient. None of the 15 normal colon mucosa samples demonstrated detectable ColoUp-1 transcript; whereas, 13 of the 15 colon cancers demonstrated an easily detectable ColoUp-1 signal (FIG. 1C).

To provide a more quantitative measurement of ColoUp-1 induction, real-time PCR analysis was performed to compare ColoUp-1 expression in a second, independent set of 29 colon cancer tumors versus matched normal colon mucosa. Again, ColoUp-1 was markedly induced in colon cancer samples, with the median tumor showing a 54-fold increased ColoUp-1 expression over normal colon mucosa, and with all 29 cancers showing a greater than 12-fold increase (FIG. 1D). Thus, these analyses validated the initial microarray finding of ColoUp-1 induction in colon cancer using both independent measurement methods and a second independent set of colon cancer samples. Methods of real-time PCR analysis of Colo-Up1 expression in these colon cancer tumors are described in Materials and Methods as: “ColoUp-1 Real-time PCR of Matched Tumor and Normal Tissues.”

### Example 3

### Detection of Endogenously Expressed ColoUp-1.

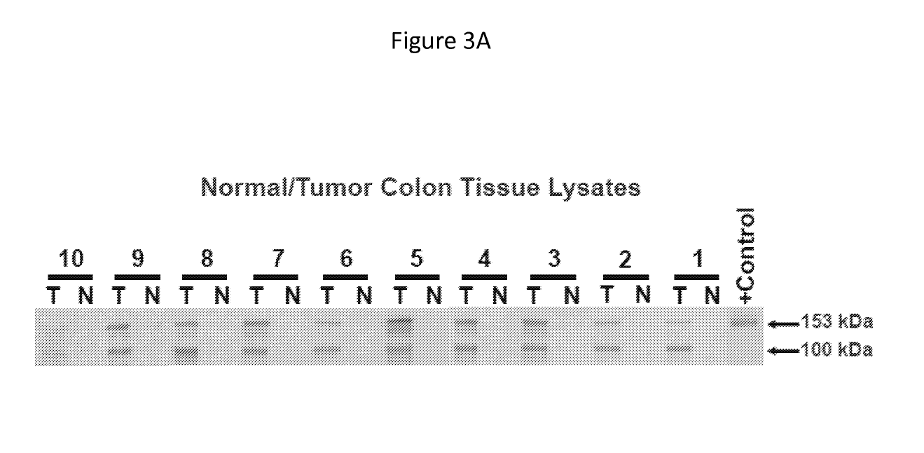

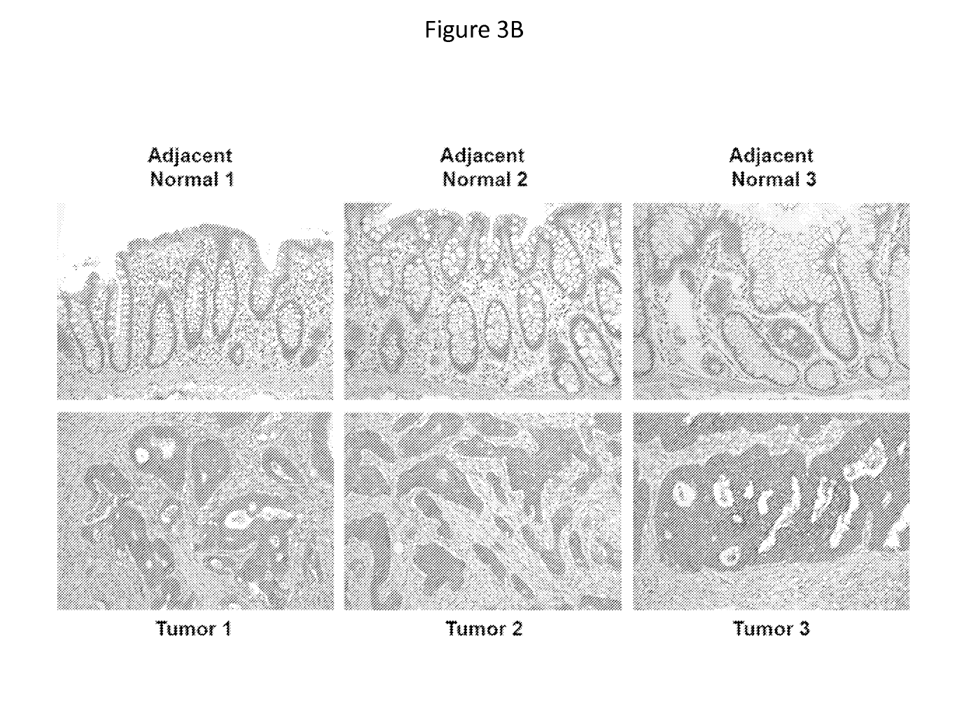

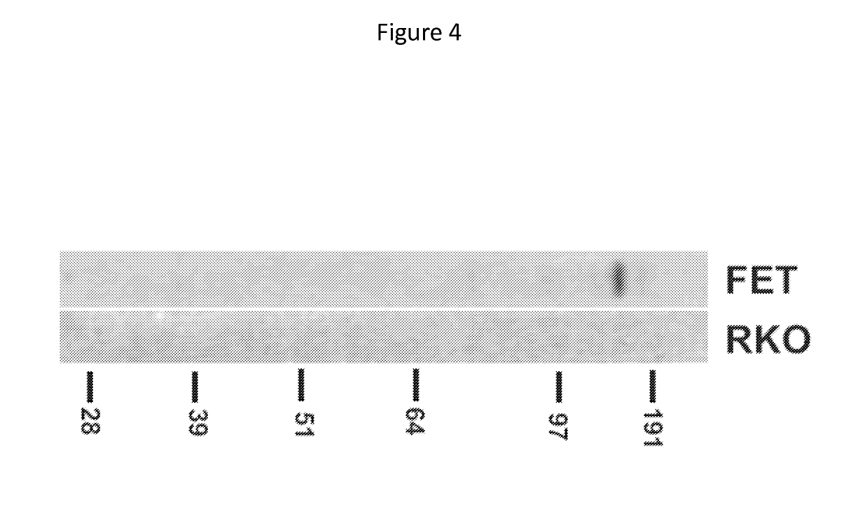

To determine if ColoUp-1 protein levels increase to match the observed increase in ColoUp-1 mRNA in colon cancer, monoclonal antibodies against purified recombinant ColoUp-1 protein were developed. The ColoUp-1 antibodies were used to characterize the endogenous ColoUp-1 protein by serial immunoprecipitation and western blot analyses on protein lysates from 10 primary colon cancer tumors versus patient's matched normal colon mucosa. This analysis confirmed that, identical to ColoUp-1 transcript, the endogenous ColoUp-1 protein is absent in normal colonic mucosa but is strongly induced in colon cancer tumors (FIG. 3A). In colon cancers, ColoUp-1 protein was detected as the expected approximately 150 kD molecular weight species along with a second 100 kD molecular weight species. The marked induction of ColoUp-1 protein in colon cancer tumors was further confirmed by immunostaining of colon cancer tissues using a ColoUp-1 specific monoclonal antibody PW-3, which demonstrated marked immunostaining of ColoUp-1 protein in malignant colon cancer cells, and absence of detectable ColoUp-1 protein in the normal colon mucosa from the same patients (FIG. 3B). The specificity of the tissue immunostaining was confirmed by demonstrating that antibody PW-3 had pronounced reactivity on immunostaining and strong identification of a single 150 kD-sized protein band on western analysis of ColoUp-1 transcript expressing FET cells; whereas, no immunostaining and no western reactivity was detected in control ColoUp-1 non-expressing RKO colon cancer cells (FIG. 4).

### Example 4

### ColoUp-1 Encodes a Glycosylated Protein.

Analysis of the ColoUp-1 protein sequence using the SignalP version 3.0 and the PSORT II algorithms both identified a putative N-terminal signal peptide sequence comprising the first 30 to 34 amino acids of ColoUp-1. Further analysis by both TMHMM and TMMOD predicted absence of any ColoUp-1 transmembrane domain. These findings suggested that ColoUp-1 may be a secreted protein. Consistent with ColoUp-1 being a secreted protein, further analysis using the NetNGlyc 1.0 and NetOGlyc 3.1 programs identified 1 potential O-linked glycosylation site and 7 potential N-linked glycosylation sites. Additionally, incubation of recombinant ColoUp-1 purified from HeLa cells with a panel of deglycosylases yielded a molecular weight shift consistent with ColoUp-1 bearing the predicted N-linked glycosylation structures (unpublished data, SPF and SM). Further analysis of the ColoUp-1 structure employing InterProScan, a portal for submitting protein sequences to be scanned for family and domain homologies by a variety of databases such as Pfam, ProDom, and SMART, resulted in the identification of only one G8 domain and two GG domains, both novel domains that have no known function. Blast analysis of the predicted ColoUp-1 protein revealed a 42% amino acid identity to human transmembrane protein-2 (TMEM2), a widely expressed protein of unknown function. ColoUp-1 homologues of unknown function were also identified in the mouse, at 91% amino acid identity, and in the rat, at 90% amino acid identity.

### Example 5

### ColoUp-1 Encodes a Secreted Protein.

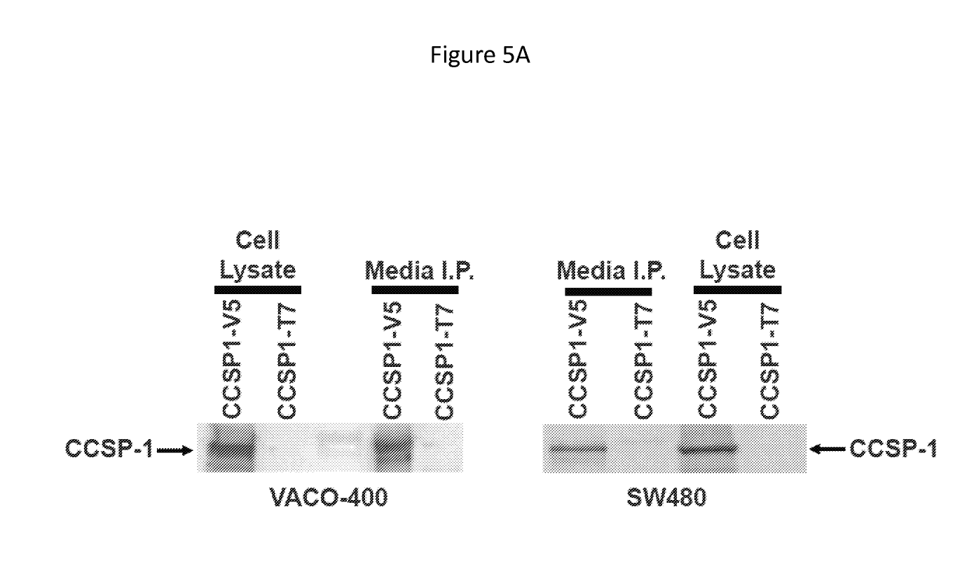

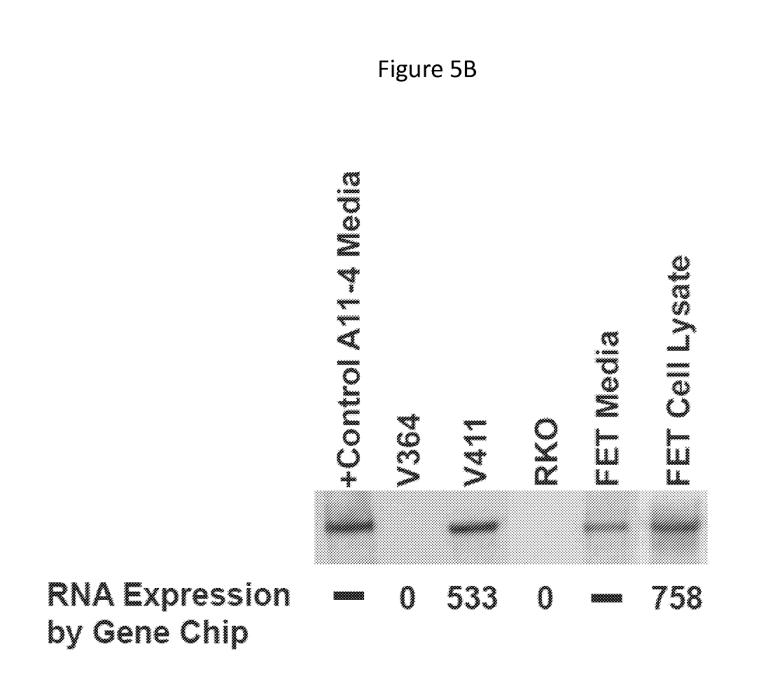

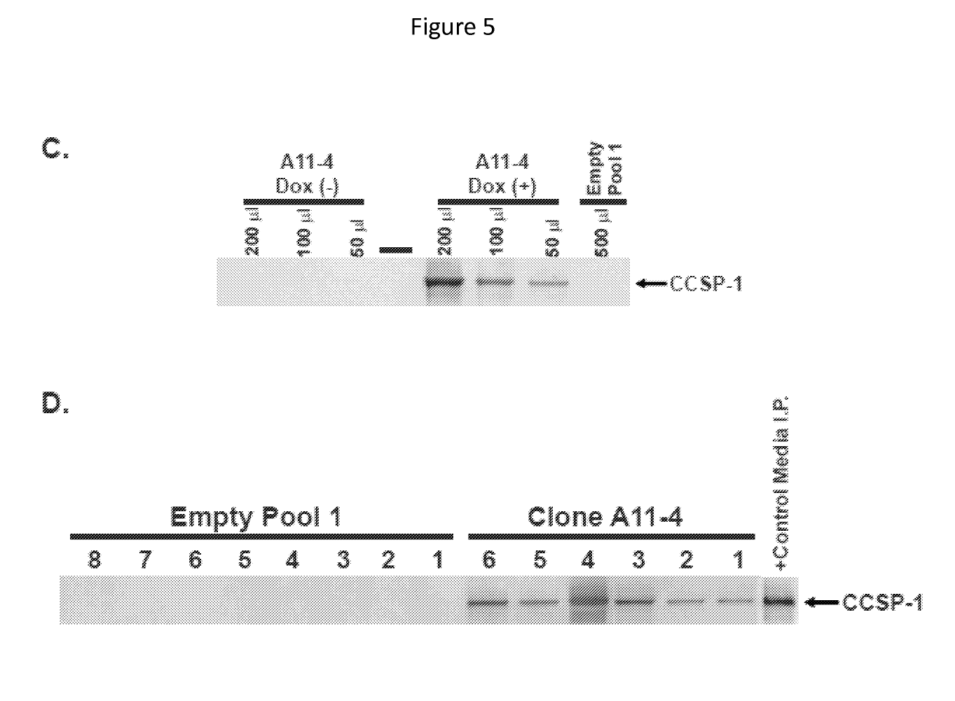

To initially test the hypothesis that ColoUp-1 is a secreted protein, SW480 and VACO-400 colon cancer cell lines were transiently transfected with expression vectors encoding ColoUp-1 fused to carboxyl terminal V5-His or T7-epitope-tags. Serial immunoprecipitation and western blot analysis using antibodies directed against the epitope tags showed these transfected cells expressed a ColoUp-1 protein of molecular weight of ˜150 kDa, with 50% of the detected ColoUp-1 protein segregating with the pelleted transfected cells, and with the remaining 50% detected in the corresponding clarified cell culture media supernatant (FIG. 5A). Cells transfected with an expression vector for a T7-epitope-tagged ColoUp-1 protein served as a negative control for assays directed against the V5-tagged ColoUp-1. Subsequent studies with anti-ColoUp-1 monoclonal antibodies resulted in the ability to easily immunoprecipitate the endogenous ColoUp-1 protein from the serum-free cell culture supernatant of colon cancer cell lines (FIG. 5B), thus confirming that the endogenous ColoUp-1 protein, like the epitope-tagged protein, is actively secreted by colon cancer cells

### Example 6

Colon Cancer Secretion of ColoUp-1 into Blood in an Animal Model.

To determine if tumor secreted ColoUp-1 would be able to enter and stably persist in the bloodstream, a mouse xenograft model was developed, based on a HeLa cell line (clone A11-4) that expresses V5-tagged ColoUp-1 under the inducible control of a doxycycline regulated promoter. To enable immunodetection of ColoUp-1 against the background of murine antibody present in mouse plasma, a “direct” immunoassay was developed, in which V5-tagged ColoUp-1 was immunoprecipitated by anti-V5 antibodies conjugated to beads, and then detected by western blot assay using anti-V5 antibodies directly conjugated to horseradish peroxidase. Assay of A11-4 cells in cell culture by the direct ColoUp-1-V5 assay demonstrated secretion of the ˜150 kDa ColoUp-1 protein into the cell culture medium of doxycycline treated cells, and its absence in these cells maintained without doxycycline treatment (FIG. 5C). A11-4 cells were then injected subcutaneously into athymic nude mice, allowed to grow for 7-9 weeks, with doxycycline treatment starting at week 3, and mouse plasma harvested. As demonstrated in FIG. 5D, epitope tagged ColoUp-1 protein was detected in plasma from every mouse bearing a ColoUp-1-V5-His expressing xenograft (6/6), and from no control mouse bearing xenografts from empty vector transfected cells (8/8). Similar results were obtained on analysis of mice bearing xenografts from a second independently derived HeLa clone also expressing a doxycycline inducible ColoUp-1-V5 fusion protein (data not shown). Thus, tumor derived ColoUp-1 can gain access to the mouse blood, can circulate as a stable product, and can be detected by immunoassay.

### Example 7

### Detection of Circulating ColoUp-1 in Human Plasma.

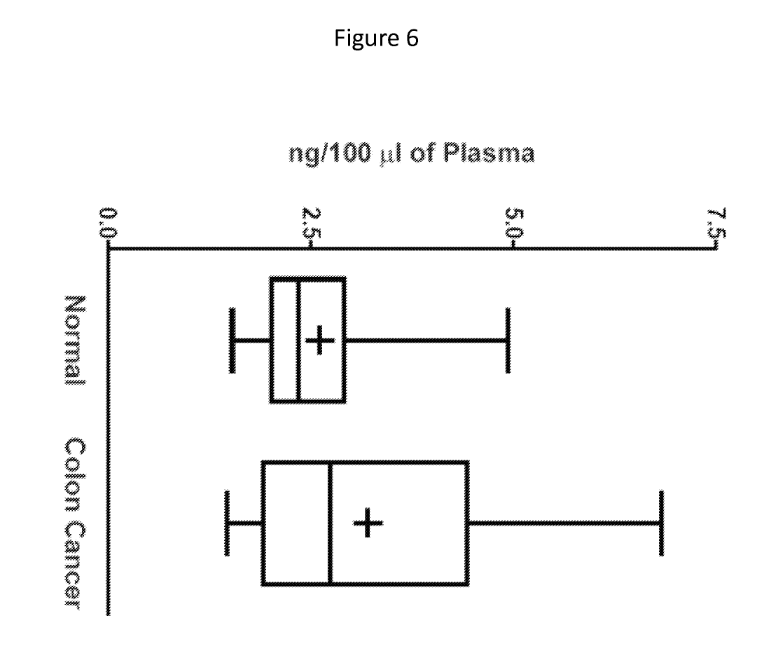

To test if ColoUp-1 protein can circulate stably in human blood, two independent ColoUp-1-directed sandwich ELISAs were developed using anti-ColoUp-1 monoclonal antibodies that on western blot assays were demonstrated as specific for recognizing ColoUp-1 protein. Circulating ColoUp-1 was detected as a normal plasma protein present in plasma samples from each of 21 normal volunteers (mean level 2.6 ng/100 μl±0.9, range 1.5-4.9). Slightly higher levels of ColoUp-1 were detected in a cohort of 17 colon cancer patients (mean level 3.2 ng/100 μl±1.6, range 1.5-6.8), though in this small sample set this increase fell short of statistical significance (P=0.08, one tailed T-test) (FIG. 6). Thus ColoUp-1 appears to serve a normal biological function as a circulating protein. The modest increase in blood levels in cancer patients may be interpreted to suggest that ColoUp-1 effects on tumor phenotype are most likely mediated locally in the tumor, at sites of highest ColoUp-1 protein concentration.

### Example 8

### Deletion of ColoUp-1 Inhibits Colon Tumor Growth in a Mouse Xenograft Model.

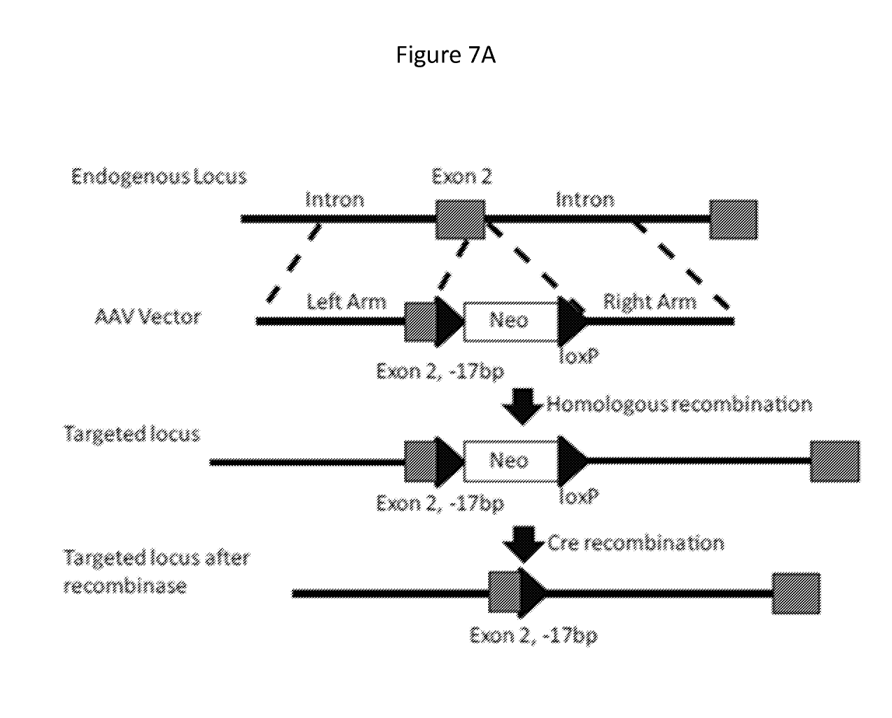

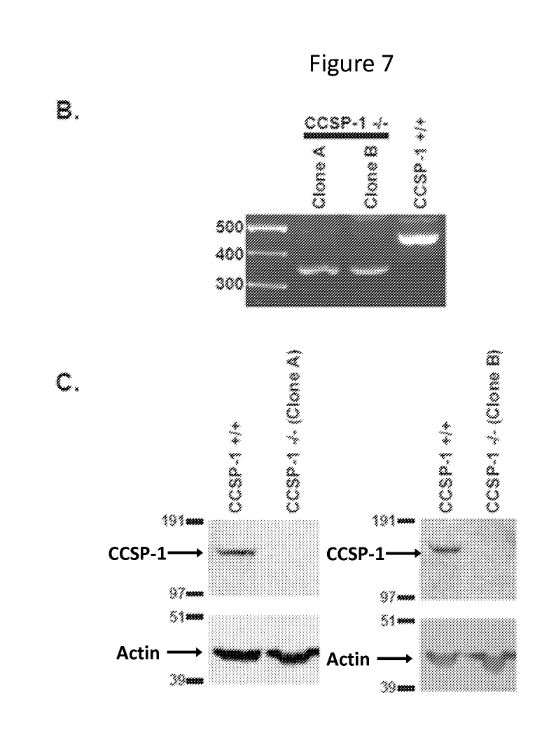

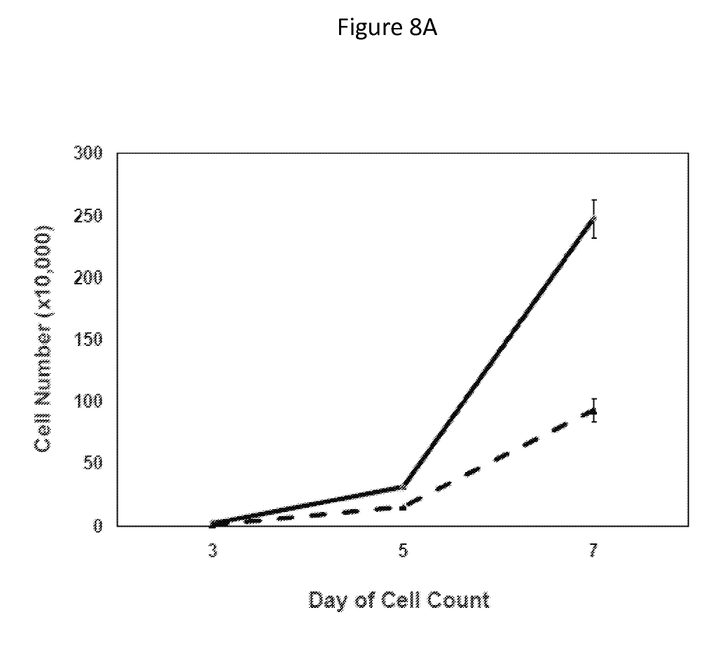

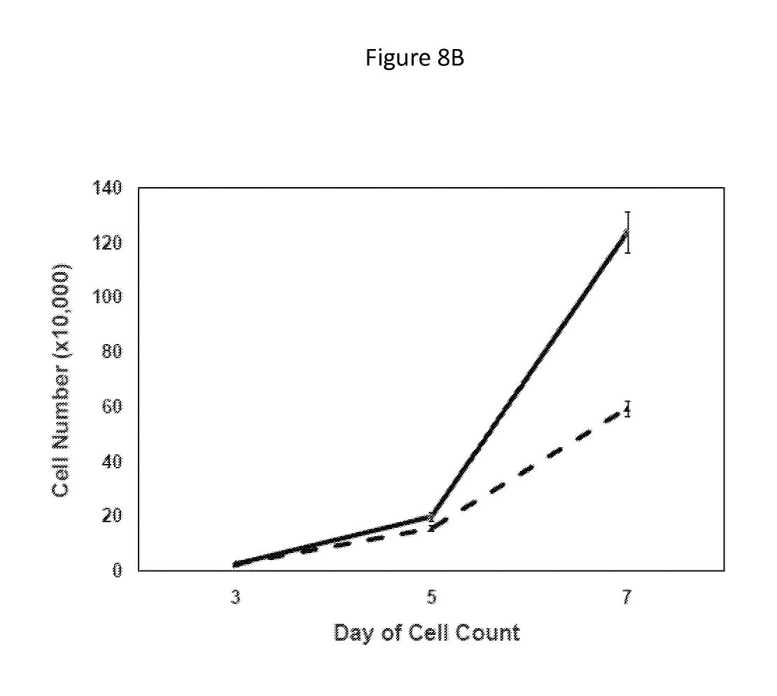

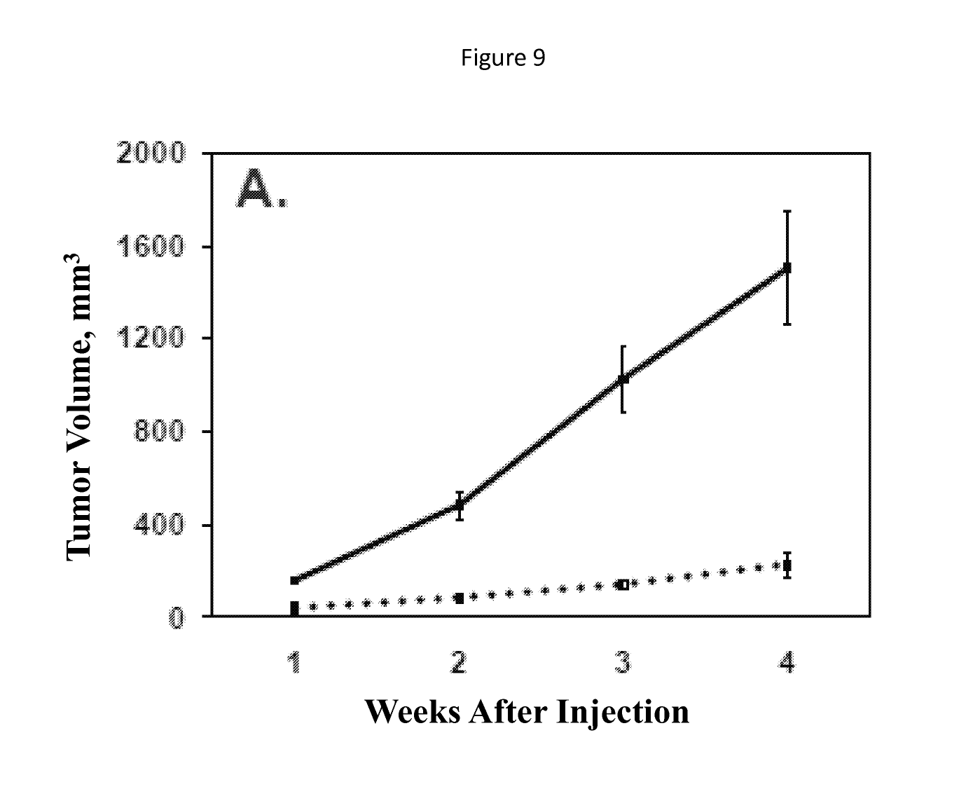

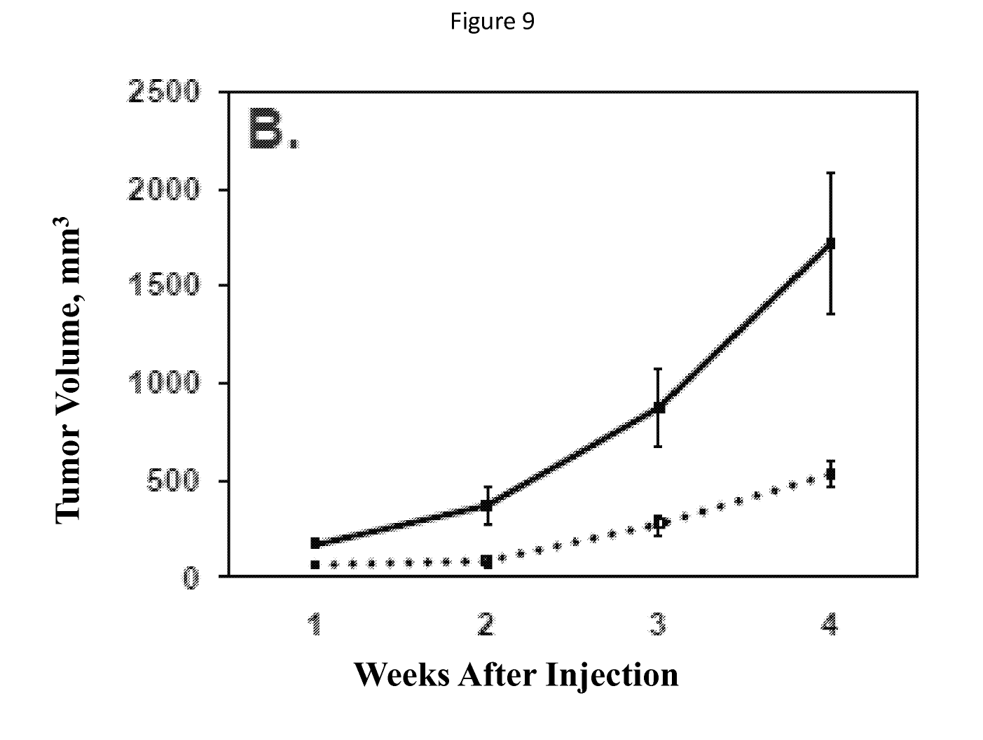

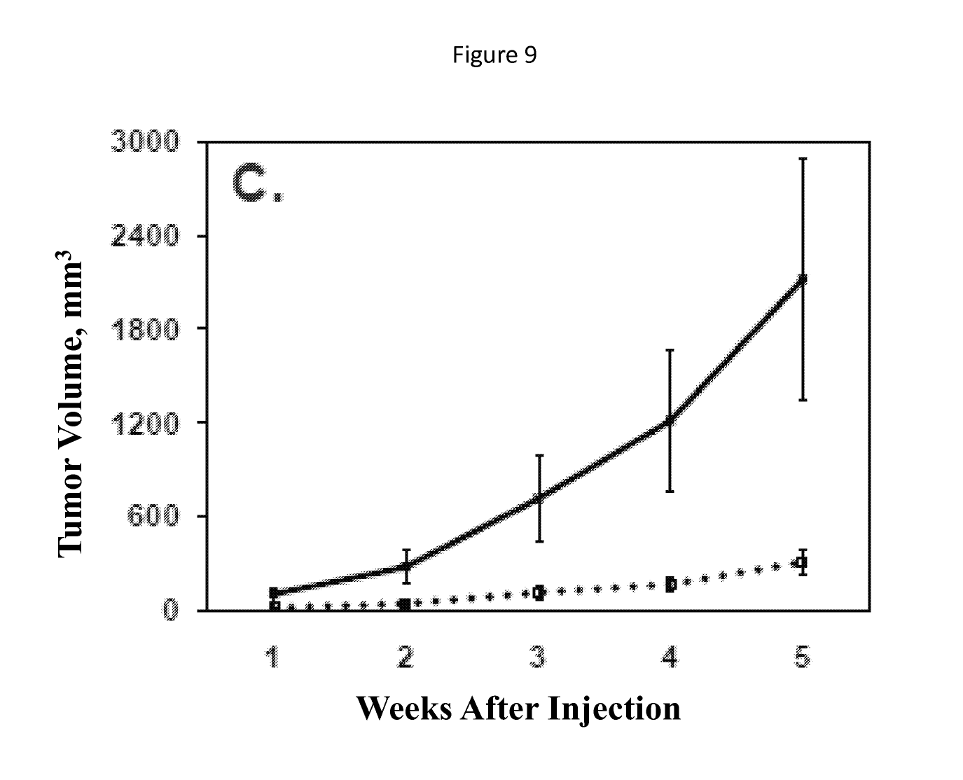

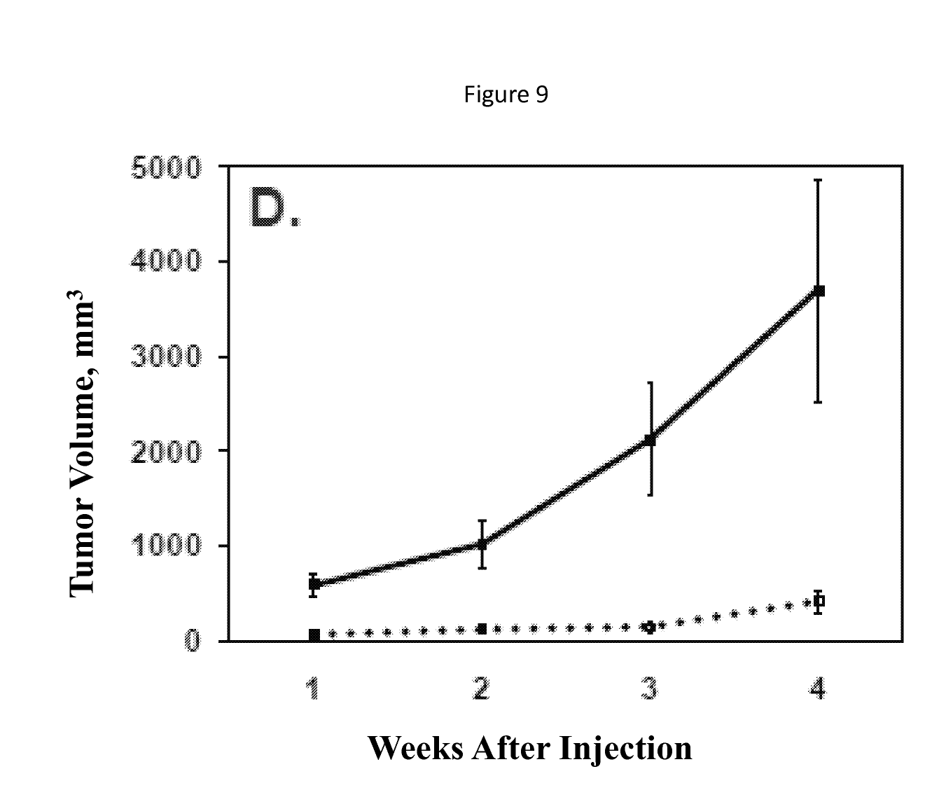

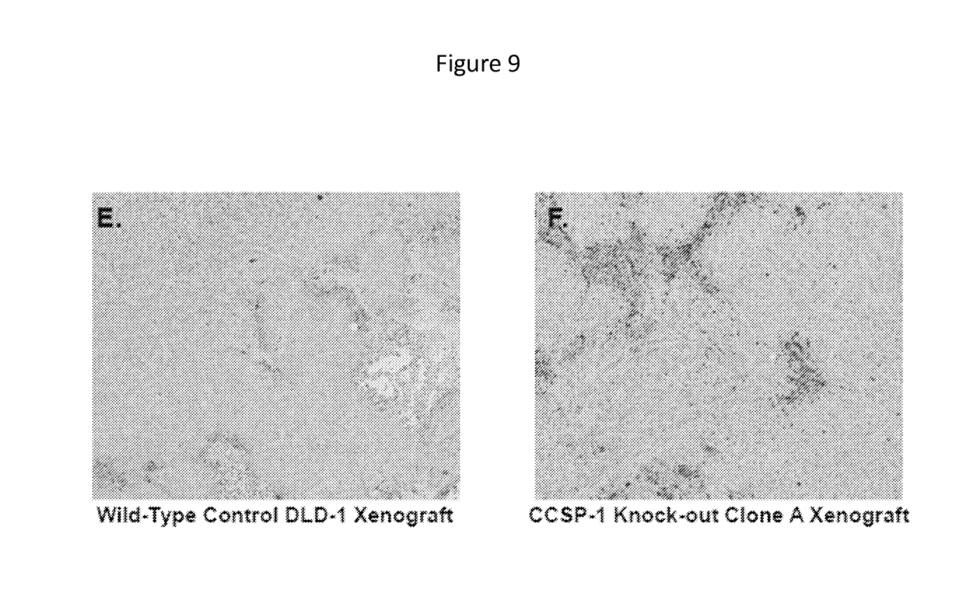

ColoUp-1 did not demonstrate focus forming activity in NIH3T3 cells, and attempts to express ColoUp-1 protein by transfection in those rare colon cancer cell lines that did not induce ColoUp-1 were in general unsuccessful. To interrogate the potential contribution of ColoUp-1 to colon cancer phenotype, gene targeting vector was used to knock out ColoUp-1 in the colon cancer diploid cell line, DLD-1 that normally expresses ColoUp-1 at high levels. A 17 bp deletion was introduced into both copies of ColoUp-1 exon 2 that contains the start ATG and signal peptide sequence. This 17 bp deletion of exon 2 (plus 2 bp of the immediately following intron) results in only the first 25 amino acids of ColoUp-1 being properly translated, with the remainder (1336 aa) being out of frame (FIG. 7A). Two independent DLD-1 clones were obtained in which both alleles of ColoUp-1 were knocked out as determined by genotyping assays, and in which no ColoUp-1 protein was detected by Western analysis (FIGS. 7B and 7C). On plastic, ColoUp-1 deleted clones showed slightly slower growth rates than wild-type cells, with numbers of ColoUp-1 deleted cells being approximately 45% that of wild-type DLD-1 at 7 days after plating (FIG. 8). The effect of deleting ColoUp-1 was however markedly amplified when the two ColoUp-1 null clones, along with control wild-type parental DLD-1 cells, were injected subcutaneously into athymic nude mice. As demonstrated in FIGS. 9A-9D, both ColoUp-1 negative DLD-1 clones grew markedly slower in mice than wild-type ColoUp-1 positive cells, with these findings replicated in duplicate experiments for each clone (Clone A, FIG. 9A-B; Clone B, FIG. 9C-D). To investigate the cause of decreased tumor growth in ColoUp-1 negative cells in vivo, ColoUp-1 expressing tumors and ColoUp-1 negative tumors were immunostained for markers of apoptosis (cleaved caspase-3), proliferation (Ki-67), immune cell infiltration (CD45), and vascularization (CD31). A marked increase in cleaved caspase-3 was detected in tumors derived from ColoUp-1 negative DLD-1 cells, suggesting that knocking out ColoUp-1 results in increased tumor apoptosis as the likely basis for the decreased tumor size in ColoUp-1 negative tumors (FIG. 9E-F). No difference was detected in Ki-67, CD45 or CD31 immunostaining when comparing ColoUp-1 expressing versus ColoUp-1 negative tumors (data not shown).

### Example 9

Patients with High Levels of Tumor ColoUp-1 Expression Have a Poor Survival.

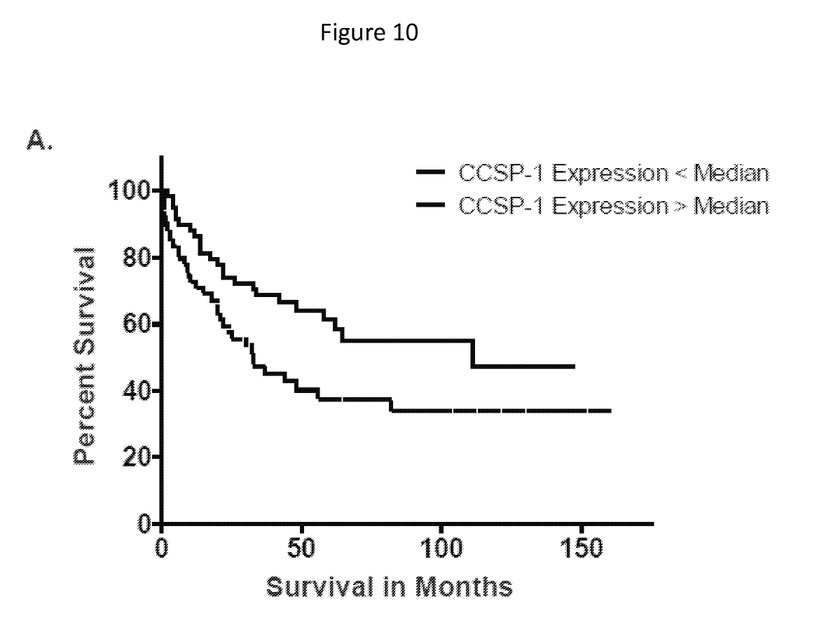

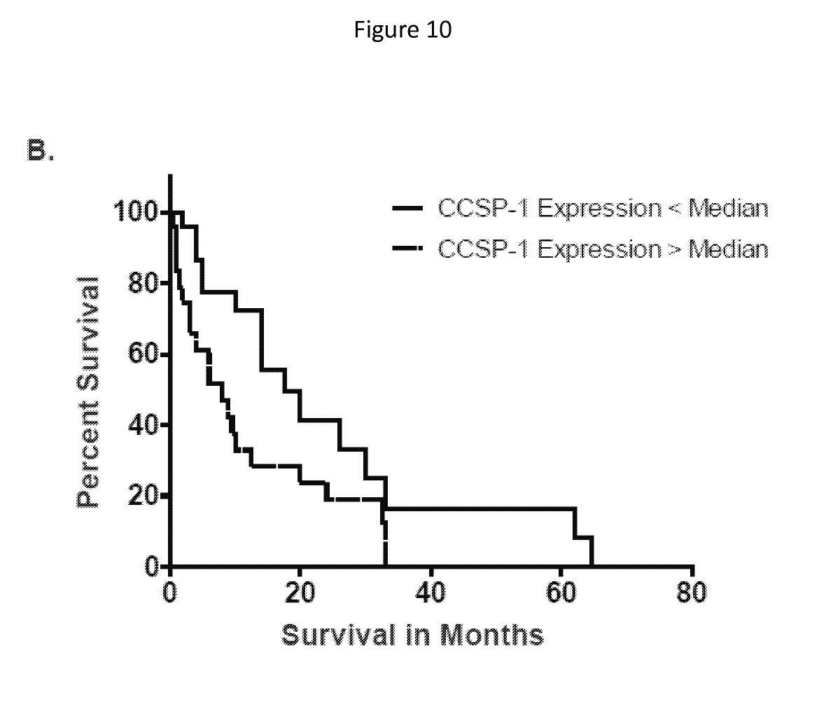

Since ColoUp-1 expression is highly up-regulated in colon cancer and appears to play a role in tumor phenotype, it was hypothesized that ColoUp-1 might be a prognostic for patient clinical outcome. ColoUp-1 mRNA expression levels were determined by gene expression analysis on Affymetrix GeneChip Human Exon 1.0 ST arrays in colon cancer tumors obtained from 121 patients for whom follow-up clinical data on clinical outcome was previously recorded. The cohort included 42 individuals with stage II disease, 31 with stage III disease, and 48 with stage IV disease. Colon cancer cases were divided into high and low ColoUp-1 expressors based on the median ColoUp-1 expression level of 330.6. Kaplan-Meier survival analysis showed that patients (n=61) whose tumors expressed levels of ColoUp-1 above the median of 330.6 had a significantly poorer median survival of 33 months as compared to the 111 month median survival for those (n=60) whose tumors expressed ColoUp-1 below the median (P=0.02) (FIG. 10A). Median ColoUp-1 levels in stage II, stage III, and stage IV cases were 258, 390, and 409 respectively, but these differences were not statistically significant (P=0.11). Moreover, Kaplan-Meier analysis of stage 4 only cases showed that in this group of stage-matched cases, individuals with tumors above the stage IV median of 409 again showed poorer median survival (8 months) than did stage IV cases with tumors below the ColoUp-1 median (17.5 months) (P=0.024) (FIG. 10B). Thus the poorer outcome associated with tumors having highest ColoUp-1 expression is at least in part independent of tumor stage.

### Example 10

Stage III Colon Cancer Patients with High Levels of Tumor ColoUp-1 Expression Have Markedly Reduced Survival.

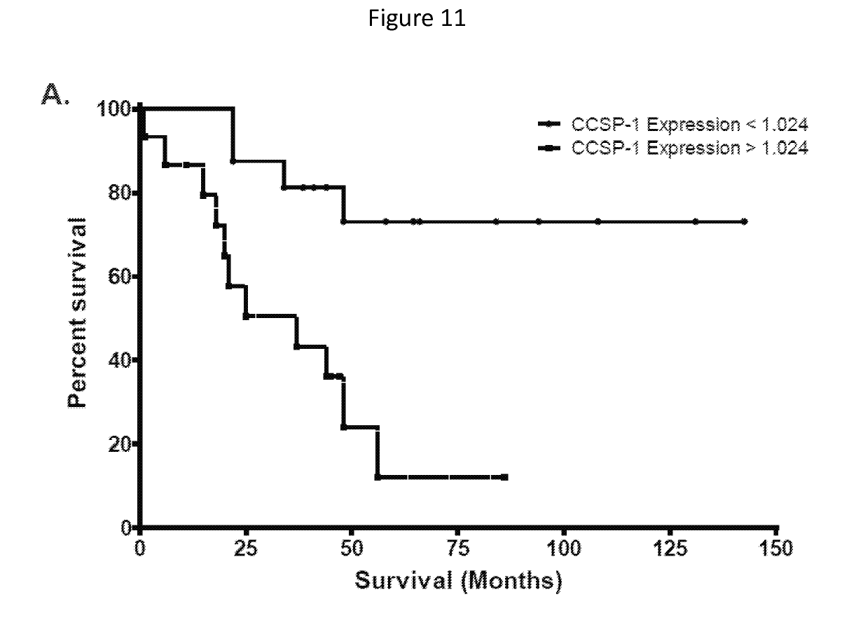

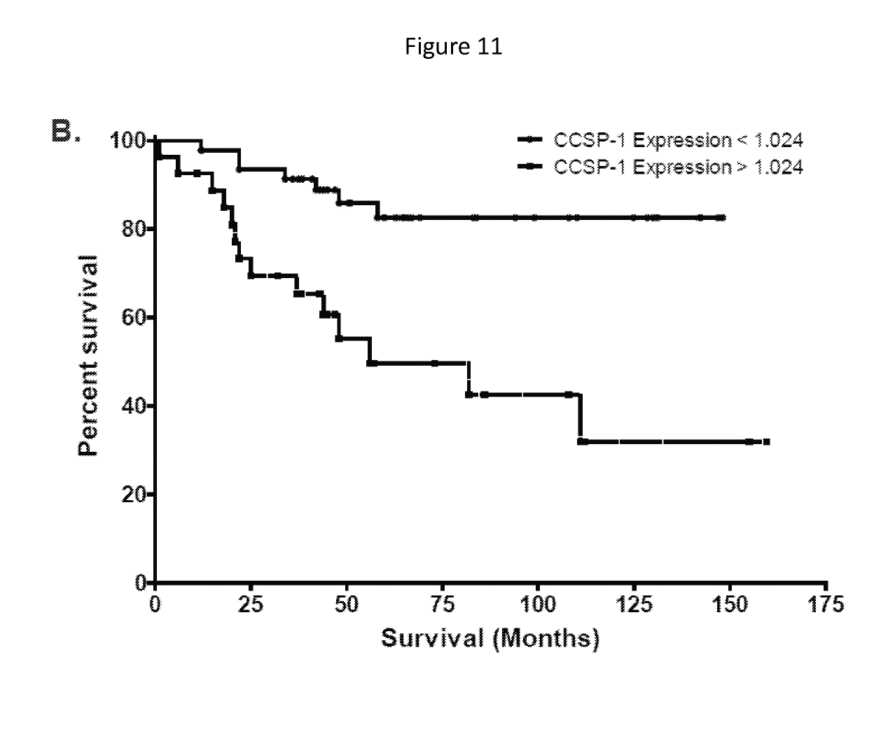

ColoUp-1 mRNA expression levels were determined by real-time PCR analysis of colon cancer tumors obtained from 31 stage III colon cancer patients for whom long term follow-up data on clinical outcome had already been recorded. Colon cancer cases were divided into those with ColoUp-1 expression greater than the median level of 1.024 (ColoUp-1 high), and those with ColoUp-1 expression lesser than the median (ColoUp-1 low). Kaplan-Meier survival analysis for colon cancer specific death showed that ColoUp-1 low cases (n=15) had notably favorable outcomes, with median survival of greater than 140 months. In contrast, ColoUp-1 high cases (n=16) demonstrated markedly worse outcomes, with median survival of only 37 months, a reduction of 8.6 years (P=0.004) (FIG. 11A). Multivariable Cox regression survival modeling was performed in order to adjust for age at diagnosis, gender, and race and showed that those with ColoUp-1 expression values greater than or equal to the median were at a 4.93 fold increased risk of death as compared to those with values below the median (HR=4.93, 95% CI=(1.50,16.14)). Kaplan-Meier and multivariable Cox regression survival models for all deaths showed similar results (data not shown). Also, essentially the same results are obtained if these stage III cases are divided according to those with ColoUp-1 expression greater than 1.047 compared to those with ColoUp-1 expression lesser that 1.047, corresponding essentially to the gap in distribution in ColoUp-1 expression values of cases near the median.

The adverse outcome associated with high ColoUp-1 tumor expression was also evident in an analysis combining the 31 stage III colon cancer cases with an additional 42 stage II colon cancer cases with similar long term follow-up. In a Kaplan-Meier survival analysis of this combined cohort of 73 colon cancers cases, patients with ColoUp-1 expression below 1.024 again showed favorable outcomes, with median survival time greater than 148 months; whereas cases with ColoUp-1 expression above 1.024 again showed a much reduced median survival time of 56 months (FIG. 11B). Moreover, in this combined cohort, there was a 10-fold increase in the level of statistical significance for the differences in outcome between ColoUp-1 high versus low groups, as compared to the effect of ColoUp-1 observed in stage III cases only (P=0.0003 for differences in stage II plus III cases versus P=0.004 for stage III cases only) (FIG. 11B). While the small number of events in the stage II cohort precludes meaningful statistical analysis of these stage II only cases, the increased significance for the survival difference in the combined stage II plus stage III group provides added support for high ColoUp-1 expression being associated with adverse outcome. Additionally, essentially the same results are obtained if these stage II plus stage III cases are divided according to those with ColoUp-1 expression greater than 1.047 compared to those with ColoUp-1 expression lesser that 1.047, corresponding essentially to the gap in distribution in ColoUp-1 expression values of cases near the stage III median value of 1.024.

Methods of real-time PCR analysis of Colo-Up1 expression in these stage II and stage III colon cancers are described in Materials and Methods as: “Analysis of ColoUp-1 expression level in Stage II and Stage III colon cancer cases.”

### Materials and Methods

Sequences. Human ColoUp-1 mRNA and gene sequence GenBank accession numbers as deposited by our group are AY581148, AY585237, and AY581149.

Cell lines and tissues. VACO cell lines were established in the investigators laboratories according to previously described methods (Science 1995; 268(5215):1336-8)]. The lines are authenticated by DNA fingerprinting against original patient tumors on an annual basis. SW480 and DLD-1 cell lines were obtained from ATCC (Manassas, Va.) and the cell lines were used for experiments with minimal passages after resuscitation. All colon cancer cell lines were maintained in MEM2+ medium except for DLD-1 which was maintained in McCoys medium with 10% FBS. The tetracycline-inducible HeLa cell line, T-REx™-HeLa was obtained from Invitrogen (Carlsbad, Calif.) and grown according to the suppliers recommended protocol. FET was a generous gift from Dr. Michael Brattain and grown as previously described. All normal colon, primary colon cancer, and liver metastasis tissues were obtained from the archives of University Hospitals of Cleveland (Cleveland, Ohio) under an IRB approved protocol.

DNA expression microarray analysis. Design of the custom expression monitoring microarrays using Affymetrix GeneChip technology as well as preparation of samples, hybridization to the microarrays, and data analysis was done as described in Cancer Res. 2002 Feb. 15; 62(4):1134-8. RNA run on Affymetrix Human Exon 1.0 ST Arrays and subsequent generation of gene expression values were generated as described in Cancer Res. 2009; 69:7577-86 and Mol Cell Proteomics. 2009; 8:827-45).

Northern blot analysis. Northern analysis was performed as previously described in Cancer Res. 2003; 63:1568-75. The probe for exons 1-9 of ColoUp-1 was amplified by PCR using the primers 5′-AGGCGTGACACTGTCTCGGCTACAG-3′ (forward) and 5′-CCACTCCACGTCTTGAACCCAC-3′ (reverse), while the probe for exons 13-22 was amplified using the primers 5′-GACCTCTCCATCCATCATACATTCTC-3′ (forward) and 5′-CCAGCCAGTTGTCATTCTTCGTG-3′ (reverse).

ColoUp-1 Real-time PCR. ColoUp-1 was amplified from cDNA using the human KIAA1199 TaqMan primer/probe kit Hs00378520_m1 (Perkin-Elmer Biosciences, Foster City, Calif.) and 1× IQ Supermix from Bio-Rad (Hercules, Calif.). The total reaction volume was 25 μl. The conditions for the amplification were as follows: 50° C. 2′×1; 95° C. 10′×1; 95° C. 15″, 60° C. 1′×50; 4° C. hold. Beta-2-microglobulin (B2M) was used as the endogenous control to allow for ColoUp-1 expression quantification. B2M was amplified using the human B2M TaqMan primer/probe kit from Perkin-Elmer and 1× IQ Supermix. The PCR cycling conditions for B2M were 50° C. 2′×1; 95° C. 10′×1; 95° C. 15″, 60° C. 1′×50; 4° C. hold. The level of ColoUp-1 expression was determined as the ratio of ColoUp-1:B2M=2exp−(CT_{ColoUp-1}−CT_{B2M}). The colon cancer cell line VACO-786 was used for a positive control, and both water only and VACO-786 RNA that had not undergone the reverse transcriptase step were used as negative controls. Serial dilutions of VACO-786 were used to create a standard curve. All PCR samples were performed in triplicate. Similar results were also obtained using a SYBR Green based real-time PCR assay where ColoUp-1 was amplified using the primers 5′-CCCAGGTTATTCAGAGCACATTC-3′ (forward) and 5′-TGGCAGAGATGATTGAGAGGAAC-3′ (reverse) and 1× IQ SYBR Green Supermix from Bio-Rad (Hercules, Calif.). The total reaction volume was 25 μl and the conditions for the amplification were as follows, 95° C. 10′×1; 95° C. 15″, 66° C. 30″, 72° C. 30″×50; 4° C. hold. Products from representative ColoUp-1 PCR reactions were sequenced to confirm that the reactions actually amplified authentic ColoUp-1 derived DNAs.

ColoUp-1 Real-time PCR of Matched Tumor and Normal Tissues. RNA from all tissue samples used was prepared by extraction with guanidine isothiocyanate as previously described (Biochemistry 1979; 18(24):5294-9). RNA concentrations were determined using a ND-1000 Spectrophotometer (NanoDrop, Wilmington, Del.) and all samples used had an A_{260/280} ratio value greater than 1.70. All reverse transcription quantitative real-time PCR assays were performed following the MIQE guidelines (Clin. Chem. 2009; 55(4):611-22). cDNA was synthesized from 1 μg of input RNA using AMV Reverse Transcriptase (Roche, Indianapolis, Ind.) following the manufactures recommended protocol and used for subsequent qPCR assays.

Real-time PCR measurement of ColoUp-1 from paired normal and tumors samples was performed using the human hydrolysis probe/primer set Hs00378520_m1 (KIAA1199, NM_{—}018689) from Applied Biosystems (Foster City, Calif.). A 20 ul reaction mix contained 1 μl of cDNA template and a 1:20 dilution of primer/probe in 1× IQ-Supermix (Bio-Rad, Calif.) and the cycling conditions were 95° C. for 4 min, followed by 50 cycles of 95° C. for 15 sec and 60° C. for 1 min. Beta-2-microglobulin (B2M) was used as the reference gene for normalization and was amplified using the human B2M (NM_{—}004048) hydrolysis probe/primer set Hs99999907_m1 from Applied Biosystems following the same reaction conditions above. The level of ColoUp-1 expression was determined as the ratio of ColoUp-1:B2M=2exp−(Cq_{ColoUp-1}−Cq_{B2M}). For each reverse transcription reaction, Cq_{ColoUp-1} and Cq_{B2M} values were determined as the average values obtained from three independent real-time PCR reactions. RNA that had not undergone the reverse transcriptase step, as well as a water sample that was carried through the reverse transcriptase step, were used as negative controls. Both controls were negative for all assays performed. PCR efficiency, R², slope, and y intercept for the calibration curve for each assay was as follows ColoUp-1 (98.7, 0.992, −3.35, 23.01) and B2M (93.2, 0.995, −3.49, 19.07). Similar results were also obtained using a SYBR Green based real-time PCR assay where ColoUp-1 was amplified using the primers 5′-CCCAGGTTATTCAGAGCACATTC-3′ (forward) and 5′-TGGCAGAGATGATTGAGAGGAAC-3′ (reverse) and 1× IQ SYBR Green Supermix from Bio-Rad (Hercules, Calif.). The total reaction volume was 25 μl and the conditions for the amplification were as follows, 95° C. 10′×1; 95° C. 15″, 66° C. 30″, 72° C. 30″×50; 4° C. hold. Products from representative ColoUp-1 PCR reactions were sequenced to confirm that the reactions actually amplified authentic ColoUp-1 derived DNAs.

Identification of a reference gene set for normalizing real-time PCR of human colon cancer samples. To identify the most stable reference transcripts for normalizing real-time PCR assays of colon cancer tumors, gene expression profiles were mined from 72 colon cancer samples that were analyzed on Affymetrix Human Exon 1.0 ST arrays. Eight transcripts were selected based on having a coefficient of variation <0.30 and uniform microarray expression that was >100 average signal intensity. Stability of these eight transcripts (DDA1, SAC3D1, TMEM160, CPNE2, TMEM134, ZNF787, ZNF746, SIRT3) was further tested by real-time PCR analysis of 28 colon cancer tumors using hydrolysis probe/primer sets from Applied Biosystems following the same reaction conditions as above. The real-time PCR results were examined using the software programs BestKeeper (Biotechnol. Lett. 2004; 26(6):509-15), Normfinder (Cancer Res. 2004; 64(15):5245-50), and genorm^{PLUS} (Genome Biol. 2007; 8(2):R19) to determine the top four stable transcripts from each program (Table 1).

| | RANK | | | |
|---|---|---|---|---|
| Program | First | Second | Third | Fourth |
| Normfinder | CPNE2 | ZNF787 | SIRT3 | TMEM160 |
| BestKeeper | TMEM134 | TMEM160 | SAC3D1 | CPNE2 |
| genorm^{PLUS} | SAC3D1 | TMEM160 | CPNE2 | DDA1 |

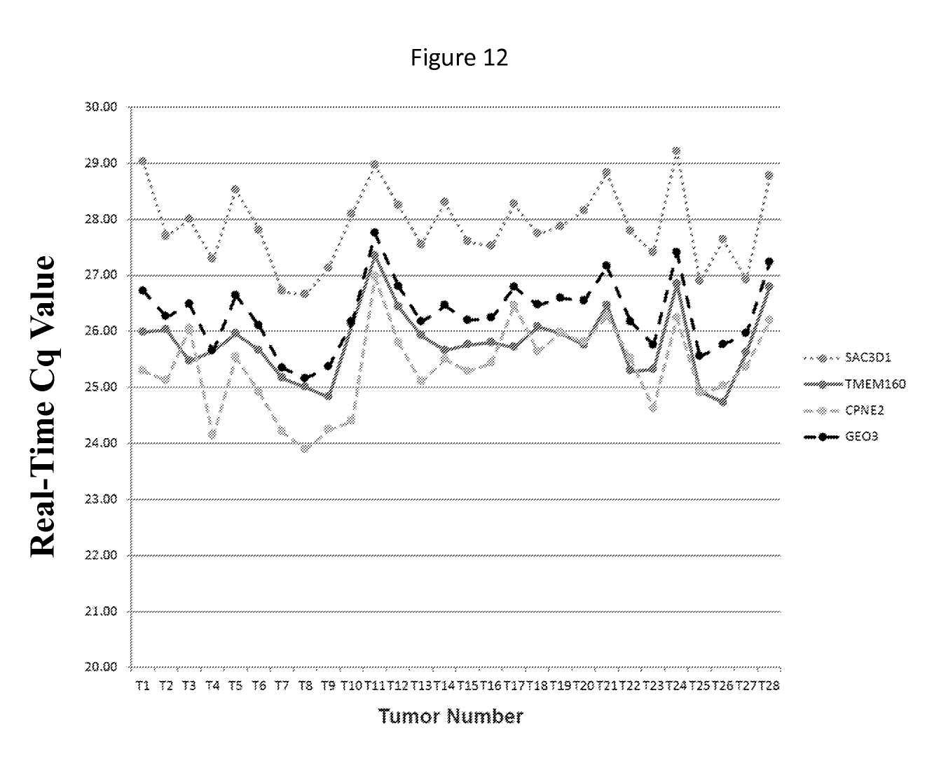

Copine II (CPNE2), SAC3 domain-containing protein 1 (SAC3D1) and Transmembrane protein 160 (TMEM160) were selected as reference transcripts based on being in the top four most stable transcript lists for all three programs (CPNE2 and TMEM160) or for two of the three programs (SAC3D1) (Supplemental Table 1). The hydrolysis probe/primer sets for CPNE2 (NM_{—}152727), SAC3D1 (NM_{—}013299), and TMEM160 (NM_{—}017854) are Hs00541611_m1, Hs01017027_m1, and Hs00215289_m1 respectively, from Applied Biosystems. FIG. 12 shows the individual Cq values of each of these transcripts, and the geometric mean of the Cq values for all three genes (Cq_{GEO3}) as determined in amplifications from 1 μg of input RNA from each of the tumors. Note that while highly stable across colon cancer tissue samples, these reference genes are not suitable as an internal standard for comparison of gene expression between normal colon tissues and colon cancers.

Analysis of Colo Up-1 expression level in Stage II and Stage III colon cancer cases. Real-time PCR measurement of ColoUp-1 from 42 stage II and 31 stage III tumor samples was performed using the human hydrolysis probe/primer set Hs00378530_m1 (KIAA1199, NM 018689) from Applied Biosystems (Foster City, Calif.) and followed the same reaction conditions as specified above. Also as above, (Cq_{GEO3}), the geometric mean of the Cq values for CPNE2, SAC3D1 and TMEM160 was used for normalization. The level of ColoUp-1 expression was determined as the ratio of ColoUp-1-1:GEO3=2exp−(Cq_{ColoUp-1}−Cq_{GEO3}). For each reverse transcription reaction, Cq_{ColoUp-1}, Cq_{CPNE2}, Cq_{SAC3D1}, and Cq_{TMEM160}, values were determined as the average values obtained from three independent real-time PCR reactions. RNA that had not undergone the reverse transcriptase step, as well as a water sample that was carried through the reverse transcriptase step, were used as negative controls. Both controls were negative for all assays performed. PCR efficiency, R², slope, and y intercept for the calibration curve for each assay was as follows ColoUp-1 (98.5, 0.999, −3.36, 23.87), CPNE2 (95.8, 0.996, −3.43, 26.45), SAC3D1 (96.1, 0.996, −3.42, 28.79), and TMEM160 (100.5, 0.979, −3.31, 28.81).

Construction of ColoUp-1 expression vectors. Full-length ColoUp-1 was amplified from cDNA and cloned into the pcDNA3.1/V5-His© TOPO® TA expression vector using the primers 5′-CGTGACACTGTCTCGGCTACAGAC-3′ (forward) and 5′-CAACTTCTTCTTCTTCACCACAG-3′ (reverse) for the V5/His-tagged construct, while the full-length, T7-tagged construct was cloned into the vector using the primers 5′-CGTGACACTGTCTCGGCTACAGAC-3′ (forward) and 5′-TCAACCCATTTGCTGTCCACCAGTCATGCTAGCCATCAACTTCTTCTTCTTCACCACAG-3′ (reverse), which contains an incorporated T7-tag sequence (underline) followed by a stop codon. For the tetracycline-inducible ColoUp-1 construct, V5/His-tagged ColoUp-1 was cut out of the pcDNA3.1/V5/His vector with KpnI and PmeI, the overhangs filled in, and blunt-end ligated into the PmeI site of pcDNA4/TO/myc-His (Invitrogen, Carlsbad, Calif.). Sequence of the tagged constructs was confirmed by sequencing both strands (Cleveland Genomics, Cleveland, Ohio).

Transfection and detection of ColoUp-1 from cell lysates and cell media. Cells were seeded at 0.8×10⁶ cells/100 mm dish (SW480) and 1.0×10⁶ cells/100 mm dish (VACO-400). The next day the cells were transfected with 2 μg of either T7- or V5/His-tagged ColoUp-1 using 12 μl of Fugene (Roche, Indianapolis, Ind.) for SW480 or 60 μl of Effectene (Qiagen, Valencia, Calif.) following the manufacturer's protocols. Seventy-two hours after transfection, the media was removed and clarified by centrifugation for 5 min at 2,000 rpm and the supernatant was collected in a fresh tube. Epitope-tagged ColoUp-1 was immunoprecipitated from media samples using either a 1:1000 dilution of mouse, anti-T7 antibody (Novagen, Madison, Wis.) or a 1:333 dilution of mouse, anti-V5 antibody (Invitrogen, Carlsbad, Calif.) and rocking overnight at 4° C. The next day Protein G beads (Upstate Biotechnology, Lake Placid, N.Y.) were added to each sample and rocked at 4° C. for 1.5 h. The samples were then washed 3 times with RIPA buffer (1× PBS, 1% Igepal CA-630, 0.5% sodium deoxycholate, 0.1% SDS) containing Complete Mini protease inhibitor cocktail (Roche, Indianapolis, Ind.) and loaded onto a 4-12% Bis-Tris SDS-PAGE (Invitrogen, Carlsbad, Calif.) for protein separation. The accompanying pellet of transfected cells were similarly lysed for 15 min at 4° C. in RIPA buffer containing protease inhibitor cocktail. Lysates were then centrifuged for 10 min at 14,000 rpm at 4° C. to remove the insoluble fraction, and the clarified supernatants were also loaded onto the SDS-PAGE gel. Equal percentage amounts of total cell lysates and total cell culture media were represented on each SDS-PAGE gel. Proteins were transferred onto Immobilon™-P PVDF membranes (Millipore, Bedford, Mass.). Membranes were blocked with 5% nonfat milk, and then incubated overnight at 4° C. with either a 1:3000 dilution of mouse, anti-V5 antibody, or a 1:3000 dilution of mouse, anti-T7 antibody. The blots were washed with PBS/0.2% Tween 20, and then incubated with a 1:1500 dilution of donkey anti-mouse horseradish peroxidase (Jackson ImmunoResearch Laboratories, Inc., West Grove, Pa.). Enhanced Chemiluminescence Plus (Amersham Biosciences, Piscataway, N.J.) and a STORM 840 phosphoimager were used to detect protein bands.

Selection of stable Colo Up-1 expressing Hela clones. T-REx™-HeLa cells were seeded at 12,000 cells/100 mm dish. The next day the cells were transfected with 20 μg of pcDNA4/ColoUp-1-V5/His plasmid construct using TransIT®-Hela Monster transfection reagent (Mirus, Madison, Wis.) following the manufacturer's protocol. Two days after transfection, pcDNA4-containing cells were selected for using 150 μg/ml of zeocin. Stable clones that grew out of the zeocin selection were confirmed for inducible ColoUp-1 expression by treatment with 1 μg/ml doxycycline for 24 h and then assayed by Western blot. The procedure for creating a pool of stable control clones was the same as above except an empty pcDNA4/TO/myc-His vector was used for transfection.

Detection of ColoUp-1 in mouse plasma. Athymic female nude mice, 4-6 weeks of age, were obtained from the Animal Core Facility of Case Western Reserve University (Cleveland, Ohio) and housed in a clean pathogen-free room. Mice were injected subcutaneously on each flank with 5×10⁶ T-REx™-Hela cells expressing an inducible V5-tagged ColoUp-1 or a control empty vector. Three weeks after injection, the regular water was replaced with doxycycline-containing water (750 μg/ml) and the water was changed twice weekly. Mice were sacrificed 7-9 weeks after injection and exsanguinated blood was collected into tubes containing 100 mM EDTA in order to prevent clotting. The tubes were spun for 10 min at 5,000 rpm (2,040×g) at 4° C. and the plasma was transferred to a new tube, whereupon 30 μl of agarose-conjugated anti-V5 antibody beads (Invitrogen, Carlsbad, Calif.) was added to the plasma and rocked overnight at 4° C. The next day the beads were washed 3 times with RIPA buffer containing protease inhibitors, and the entire sample was loaded onto a 4-12% Bis-Tris, SDS-PAGE gel for protein separation. Western blotting was the same as above except that the primary antibody was a horseradish peroxidase-conjugated anti-V5 antibody (Invitrogen, Carlsbad, Calif.). All animal handling was in accord with institutional guidelines for animal care and research.

Purification of recombinant ColoUp-1. T-REx™-HeLa cells expressing the inducible V5/His-tagged ColoUp-1 were seeded into 1700 cm² expanded surface roller bottles (Corning Inc., Corning, N.Y.) and grown to ˜75% confluence, whereupon fresh media was added that contained 1 μg/ml doxycycline and low IgG serum. After 72 h, the media was collected and centrifuged for 5 min at 2,000 rpm to remove any cellular debris. The media was then frozen and sent to Roche Protein Expression Group (RPEG) (Roche, Indianapolis, Ind.) for purification of the V5/His-tagged ColoUp-1. Briefly, ColoUp-1 was purified by a two-step process which was comprised of a Ni-affinity step to bind the 6× His tag of ColoUp-1 and then a size exclusion chromatography step using a Sephadex G-200 column. Aliquots of the collected fractions were run on an SDS-PAGE gel and analyzed by Coomassie blue staining. The ColoUp-1-containing fractions were pooled and a concentrated using a Centricon YM-10 centrifugal filter (Millipore, Billerica, Mass.), and the protein concentration was determined using the Bradford assay. To determine the purity of the ColoUp-1 protein, a sample was run on an SDS-PAGE gel and densitometry was performed using an AlphaImager (Alpha Innotech, San Leandro, Calif.). Identity of the ColoUp-1 band was confirmed both by mass spectrometry and by western blotting with an anti-6× His-tag antibody (Roche, Indianapolis, Ind.).

Generation of anti-ColoUp-1 monoclonal antibodies. Generation of anti-ColoUp-1 monoclonal antibodies was performed under contract by Celliance Corporation (Norcross, Ga.). Briefly, 25 μg of purified recombinant protein was added to 200 μl of Ribi or Complete Freund's Adjuvant and then injected sub-cutaneously into female Balb/c mice. Supernatants from the hybridomas were first screened for anti-ColoUp-1 activity by ELISA using purified V5/His-tagged ColoUp-1. Medias positive by ELISA for anti-ColoUp-1 activity were then further screened for endogenous ColoUp-1 western blot activity using purified T7-tagged ColoUp-1 and FET cell lysates, as well as screened for immunoprecipitation activity using media collected from ColoUp-1 expressing (FET and V411) and non-expressing (V364 and RKO) cell lines. Hybridomas that tested positive for anti-ColoUp-1 activity were injected into mice and the monoclonal antibodies were purified from the ascites using Protein G beads from Roche following the manufacture's protocol.

Western analysis of native ColoUp-1 protein. Native ColoUp-1 was detected by serial immunoprecipitation and western blot using anti-ColoUp-1 monoclonal antibody B-1.2.A2 or by immunostaining using anti-ColoUp-1 monoclonal antibody PW-3. Cells were seeded at 6.0×10⁶ (FET), 1.2×10⁶ (RKO), 2.0×10⁶ (VACO-411), and 1.3×10⁶ (VACO-364) cells/100 mm dish and grown to ˜60-75% confluence. The media was then changed, and the cells were grown for another 72 h before harvesting the cells and media Immunoprecipitation and western blot analysis of native ColoUp-1 from cell line media was the same as above except that the media was precleared with 40 μl of Protein G beads by rocking at 4° C. for at least 2 hr, a 1:40 dilution of antibody supernatant was used for the overnight immunoprecipitation, and the primary antibody used for blotting was a 1:40 dilution of antibody supernatant. For the detection of native ColoUp-1 from FET and RKO cell lysates, the cell lysis and western blot analysis are the same as mentioned previously except that the primary antibody used for blotting was a 1:40 dilution of antibody supernatant.

Protein lysates from frozen human tissues were obtained by pulverizing a sample in a chilled metal tissue pulverizer and scraping the powder into chilled Pierce T-PER® lysis buffer (Pierce, Rockford, Ill.) containing both protease and phosphatase (Sigma, St. Louis, Mo.) inhibitors. The samples were then incubated for 20 min at 4° C. and were pipetted several times to ensure complete lysis. Finally, the samples were centrifuged for 5 min at 10,000 rpm and the clarified supernatants were aliquoted into fresh, chilled tubes and then stored at −80° C. The immunoprecipitation/western blot analysis was the same as for the detection of ColoUp-1 from cell line media, except that 1.0 mg of protein was used for the colon normal and tumor samples.

ELISA for ColoUp-1 from human plasma. Plasma isolated from 20 normal subjects and 17 colon cancer patients were assayed for ColoUp-1 using anti-ColoUp-1 monoclonal antibodies in sandwich ELISA as follows. 100 μl of capture antibody (2 μg/ml) was incubated per well of a 96-well ELISA overnight at 4° C. The next day the antibody was removed and the wells were blocked for 2 h at room temperature with StartingBlock (Thermo Scientific, Rockford, Ill.). After blocking, the wells are washed 3 times with wash buffer (0.5% Tween-20, 0.5M NaCl, pH 5.0) and then the 115 μl of plasma is added to each well and incubated for 3 h at room temperature. Follow incubation, the wells are washed 4 times with wash buffer and 100 μl of detection antibody (0.4 μg/ml) is added per well and incubated overnight at 4° C. The following day, the wells are washed 4 times with wash buffer and 100 μl of a 1:1000 dilution of Streptavidin-HRP (BD Pharmingen, San Diego, Calif.) for 1 h at room temperature. The wells are then washed 5 times with wash buffer and then 100 μl of TMB substrate is added to each well and is allowed to incubate for 15 min before stopping the reaction with 100 μl of 2M sulphuric acid. The wells are then read on an Envision 2103Multilabel Reader (PerkinElmer, Waltham, Mass.) at 450 nm.

Construction of ColoUp-1 deleted DLD-1 cells. Construction of the targeting vector and procedure for knocking out ColoUp-1 using a recombinant adeno-associated virus system were performed as described in Nat Methods. 2008; 5:163-5.

ColoUp-1 knock-out xenograft studies. Athymic female nude mice, 4-6 weeks of age, were injected subcutaneously on each flank with 5×10⁶ ColoUp-1 negative DLD-1 cells or the control parental DLD-1 cells (n=5 mice for each condition). Mice were sacrificed 4-5 weeks after injection and the tumors were isolated, formalin-fixed paraffin-embedded, and sectioned for immunostaining.

Immunostaining for Ki-67, CD31, CD45, and cleaved caspase-3. The antibodies Ki-67 (Dako, Carpenteria, Calif.), Cleaved Caspase-3 (Cell Signaling, Danvers, Mass.), CD31 (Abcam, Cambridge, Mass.), and CD45 (R&D Systems, Minneapolis, Minn.) were used for immunostaining. Immunostaining was similar as described above for ColoUp-1 except for the following changes. Antigen retrieval was performed by steaming at 98.5° C. for 20 min in 10 mM citrate buffer (pH 6.0), nonspecific protein blocking was performed for 20 min, and the antibodies were diluted in Serum-Free Protein Block (Dako). Antibody dilution and incubation times were as follows, Ki-67, 1:50 dilution at room temperature for 1 h, CD31 and CD45, 1:100 dilution at room temperature for 30 min, and cleaved caspase-3, 1:100 dilution at 4° C. overnight. After primary incubation, the slides were washed and Envision™+HRP Anti Mouse kit (Ki-67) or Envision™+HRP Anti Rabbit kit (CD31 and cleaved caspase-3) (Dako) was used for development, applying secondary antibody conjugated to a polymer-HRP, following manufacturer's protocol. Development times were 5 min (CD31 and cleaved caspase-3) or 10 min (Ki-67). For CD45 detection, after primary incubation the slides were, washed then incubated with an anti-rat secondary antibody (BD Pharmingen, San Diego, Calif.) at a 1:50 dilution for 30 min at room temperature, washed, incubated with Streptaviden-HRP for 30 min at room temperature, washed, and then incubated with substrate-chromogen for 5 min.

Statistical Analysis. Analysis of Kaplan-Meier survival plots was performed using the Log-rank (Mantel-Cox) test with the GraphPad Prism (La Jolla, Calif.) analysis software.

ColoUp-1 immunohistochemistry. Five μM-thick formalin-fixed paraffin-embedded tissue sections were baked at 60° C. for 75 min, deparaffinized, and rehydrated. Antigen retrieval was performed by steaming at 98.5° C. for 5 min in 10 mM citrate buffer (pH 6.0), plus a cool-down period of 20 min. Reduction of peroxidases was accomplished by incubating in 3% H₂O₂ in water for 30 min at room temperature. Nonspecific protein blocking (Serum-Free Protein Block, Dako, Carpenteria, Calif.) was performed for 60 min. Monoclonal antibodies from hybridomas that were positive for anti-ColoUp-1 activity were purified from mouse ascites and screened to identify those reactive against ColoUp-1 in an immunohistochemical assay. One such antibody, PW-3, was identified that stained cell pellets from FET colon cancer cells that express endogenous ColoUp-1, but did not stain cell pellets from a non-expressing colon cancer cell line (RKO) (data not shown), and that further identified only a single protein band corresponding to ColoUp-1 on western analysis of FET cells (Supplementary FIG. 2). The antibody was diluted (1:150) in 1% BSA (Roche) and incubated overnight at 4° C. in humidified chambers. The slides were washed thoroughly, and Protein Block was added again for 30 min. Envision™+HRP Anti Mouse kit (Dako, Carpenteria, Calif.) was used for development, applying secondary antibody conjugated to a polymer-HRP, following manufacturer's protocol. Staining was performed with diaminobenzidine (DAB)+substrate-chromogen (Dako, Carpenteria, Calif.), which was added to the slides for 7 min. All washes were done with TBST (50 mM Tris·HCl, 150 mM NaCl, 0.05% Tween, pH 7.6) diluted in deionized water. The sections were then counterstained by using Harris modified hematoxylin stain (Fisher Scientific, Pittsburgh, Pa.) for 1 min, dried and mounted.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method of determining colon cancer prognosis comprising:
measuring the ColoUp-1 expression level in a biological sample obtained from a colon cancer patient; and
comparing said ColoUp-1 expression level to a median ColoUp-1 expression level in a population of colon cancer patients;
wherein a ColoUp-1 expression level above the median ColoUp-1 expression level is indicative of poor survival prognosis, and wherein a ColoUp-1 expression level below the median ColoUp-1 expression level is indicative of good survival prognosis.

2-116. (canceled)

117. A method of treating a colon cancer patient comprising:
providing a treatment regimen to the patient;
measuring the ColoUp-1 expression level in a biological sample obtained from the patient receiving a treatment regimen;
comparing said ColoUp-1 expression level to a median ColoUp-1 expression level in a population of colon cancer patients; and
modifying the treatment regimen when the ColoUp-1 expression level in the biological sample obtained from the patient is above the median ColoUp-1 expression level.

118-124. (canceled)

125. The method of claim 117, wherein the biological sample is a tumor sample.

126. The method of claim 117, wherein the biological sample is a stool sample.

127. The method of claim 117, wherein the biological sample is whole blood or a fraction thereof.

128. (canceled)

129. The method of claim 117, wherein the biological sample is a urine sample.

130. The method of claim 117, wherein measuring ColoUp-1 expression level comprises measuring ColoUp-1 mRNA.

131. (canceled)

132. The method of claim 130, wherein the ColoUp-1 mRNA comprises a nucleic acid encoding a polypeptide comprising an amino acid sequence that is at least 95% identical to the amino acid sequence as set forth in any one of SEQ ID No: 1, SEQ ID No: 2 and SEQ ID No: 3.

133-136. (canceled)

137. The method of claim 117, wherein the population of colon cancer patients comprises patients afflicted with Stage II through IV colon cancer.

138. (canceled)

139. The method of claim 137, wherein the median ColoUp-1 mRNA expression level is in the range of 250-800.

140-141. (canceled)

142. The method of claim 139, wherein the median ColoUp-1 mRNA expression level is in the range of 325 to 400.

143. (canceled)

144. The method of claim 142, wherein the median ColoUp-1 mRNA expression level is 330.

145. The method of claim 130, wherein ColoUp-1 mRNA level is measured by real-time PCR.

146. The method of claim 145, wherein the ColoUp-1 mRNA level is normalized against the expression levels of one or more reference transcripts.

147. The method of claim 146, wherein the one or more reference transcript is selected from the group comprising: DDA1, SAC3D1, TMEM160, CPNE2, TMEM134, ZNF787, ZNF746, or SIRT3.

148-161. (canceled)

162. The method of 117, wherein measuring ColoUp-1 expression level comprises measuring ColoUp-1 protein level.

163-178. (canceled)

179. The method of claim 117, wherein the modification of said treatment regimen comprises increasing the amount or level of treatments already administered to the patient in said treatment regimen.

180. The method of claim 117, wherein the modification of the treatment regimen comprises increasing the dosage of chemotherapy or the frequency of dosages of chemotherapy.

181. The method of claim 117, wherein the modification of the treatment regimen comprises increasing the amount of radiation therapy or the frequency of administration of radiation therapy.

182. The method of claim 117, wherein the modification of the treatment regimen comprises switching from one type of treatment regimen to another type of treatment regimen.

183. The method of claim 117, wherein the modification of the treatment regimen comprises utilizing a form of treatment for the patient in addition to the treatment regimen.

184-227. (canceled)
